SEQUENCE LISTING (31 sequences)

SEQ ID NO: 1 - protein, 329 aa, Haematococcus pluvialis
MQLAATVMLEQLTGSAEALKEKEKEVAGSSDVLRTWATQYSLPSEESDAARPGLKNAYKPPPSDTKGITMALAVIGSWAAVFLHAIFQIKLPTSLDQLHWLPVSDATAQLVSGSSSLLHIVVVFFVLEFLYTGLFITTHDAMHGTIAMRNRQLNDFLGRVCISLYAWFDYNMLHRKHWEHHNHTGEVGKDPDFHRGNPGIVPWFASFMSSYMSMWQFARLAWWTVVMQLLGAPMANLLVFMAAAPILSAFRLFYFGTYMPHKPEPGAASGSSPAVMNWWKSRTSQASDLVSFLTCYHFDLHWEHHRWPFAPWWELPNCRRLSGRGLVPA

SEQ ID NO: 2 - protein, 57 aa, Pisum sativum
MASMISSSAVTTVSRASTVQSAAVAPFGGLKSMTGFPVKKVNTDITSITSNGGRVKC

SEQ ID NO: 3 - DNA, 1158 bp, Artificial Sequence
  synthetic fragment of TP-RbcS and HP BKT
atggcatctatgattagttctagcgctgtgactacagttagtcgggcatctacagttcaaagtgcggctgtagcaccttttggtggactcaagtcaatgactgggtttcccgtgaagaaagtcaacactgacatcacttcgataactagcaatggtggtcgcgtgaaaatgcagttggcagccacagttatgttagaacaactcaccggttccgctgaggcattgaaagaaaaagaaaaggaagtagcagggagttcagatgtacttaggacatgggctactcagtattcactgccatcagaagaatcagatgcggctagaccaggtctgaagaatgcgtacaagccacctccgtcggacacaaagggcattacaatggcattagcagtgattggtagctgggctgctgtctttctccatgccatttttcaaatcaaactgcctacgtctttagatcagctacattggcttcccgttagtgatgcgacggcacaacttgtttcaggatcatcctctttgcttcatattgttgtcgttttctttgtgcttgagtttctctatacgggactgtttatcactacccatgatgcaatgcacggcactatcgccatgcggaaccgtcaactcaatgatttccttggtagagtttgcatatctctctatgcttggtttgactacaacatgctacacaggaaacattgggaacatcacaatcatacaggggaggtgggtaaagatcccgatttccatagagggaacccagggattgttccttggtttgcttcattcatgagttcttatatgagcatgtggcaatttgccagacttgcctggtggacagttgtcatgcagttgctcggtgctccaatggctaatctactggttttcatggctgctgcacctatactttcagctttccgacttttctactttggaacctatatgcctcacaaaccggaacctggtgcagcctcaggctctagtcctgcggtaatgaactggtggaagtcccgtacctcgcaagcatccgacttagtgtcttttctaacctgttaccattttgatttgcattgggagcaccatagatggccattcgctccttggtgggagcttccgaattgcagaaggcttagcggacgaggtcttgttccagcttag

SEQ ID NO: 4 - DNA, 4470 bp, Artificial Sequence
  VC-SIW122-13
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtccatgattagccaagcttgcatgccgtcgaccagatctgatatctgcggccgcctcgagcatatgctagaggatccccgggtaccagcctgcttttttgtacaaacttgggtaccgagctcggatccactagtaacggccgccagtgtgctggaattcgcccttataagcttaggctcgtgtcctctttgcaaccgtccgatcattgagactcttgacatattttgaaaccaatcgaatggctgtaaattctccatctaacacaaagtttttttacagtttgttgcatttcgtttgttatggaaaagtgatgtaaatttcaaaagaaaaaaaatacaagagattctgttaacaaggcccatttttagtcaattataagcccaataaacaaatagtttggacgtttcctctcaatcgcgctttccaaaacgctgtcaagatatttatcgcatggtagcagatctcgaccgttaaaggaccaaagtagttggcaaactgagcgataatcagaaaacggccagacaatagtacactgccgaagtttctagcatcggtgtcatttgcatggcgcctaattctcgtggagaagtagtagagataataaagagcaactttcctaaacaatgatttctgtctatccctaaatatatttttttttgttttttgcagagaaactttctttttcgtttattagtactttccatgtgcatacagaaacaatgtattttgtttatttcaaaatttcgcaaagtacaaaagcatttaaagggcattatgtaccattaatatataacaaaagacaacaatttaatgagctctatgatttcagtttgaagatataatcatggcattgaatcacatcgtgattataggttacgattatgtgatttttctgtgtgtaaaattgcactcctagtttcagaatccctgactaatcctttatgctttttgaaaattagccagcatgtgtcaaattggattttcttgccataaacaagagccaaaaagcccaaaacaattatcgcctcacaccacgatgcttaagatgtctgaatatcgtcctttgatcttaccaatttatgtggctaacatttattacccctcgtgatttggtttgagattacgccatttgccaaatcagcagtaatgatggctataaattagtagtcagtgtatgtacatttttcttgttcactagcttcgagtcgactgcaattcatacagaagtgagaaaaatggcatctatgattagttctagcgctgtgactacagttagtcgggcatctacagttcaaagtgcggctgtagcaccttttggtggactcaagtcaatgactgggtttcccgtgaagaaagtcaacactgacatcacttcgataactagcaatggtggtcgcgtgaaatgtatgcagttggcagccacagttatgttagaacaactcaccggttccgctgaggcattgaaagaaaaagaaaaggaagtagcagggagttcagatgtacttaggacatgggctactcagtattcactgccatcagaagaatcagatgcggctagaccaggtctgaagaatgcgtacaagccacctccgtcggacacaaagggcattacaatggcattagcagtgattggtagctgggctgctgtctttctccatgccatttttcaaatcaaactgcctacgtctttagatcagctacattggcttcccgttagtgatgcgacggcacaacttgtttcaggatcatcctctttgcttcatattgttgtcgttttctttgtgcttgagtttctctatacgggactgtttatcactacccatgatgcaatgcacggcactatcgccatgcggaaccgtcaactcaatgatttccttggtagagtttgcatatctctctatgcttggtttgactacaacatgctacacaggaaacattgggaacatcacaatcatacaggggaggtgggtaaagatcccgatttccatagagggaacccagggattgttccttggtttgcttcattcatgagttcttatatgagcatgtggcaatttgccagacttgcctggtggacagttgtcatgcagttgctcggtgctccaatggctaatctactggttttcatggctgctgcacctatactttcagctttccgacttttctactttggaacctatatgcctcacaaaccggaacctggtgcagcctcaggctctagtcctgcggtaatgaactggtggaagtcccgtacctcgcaagcatccgacttagtgtcttttctaacctgttaccattttgatttgcattgggagcaccatagatggccattcgctccttggtgggagcttccgaattgcagaaggcttagcggacgaggtcttgttccagcttagggatccactagtgattgcggccgctagactatactatgttttagcctgcctgctggctagctactatgttatgttatgttgtaaaataaacacctgctaaggtatatctatctatattttagcatggctttctcaataaattgtctttccttatcgtttactatcttatacctaataatgaaataataatatcacatatgaggaacggggcaggtttaggcatatatatacgagtgtagggcggagtggtttatcagatctggtcgacggcatgcaagcttggcgtaatcatggcaacttttctatacaaagttggatggcatgcaagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtc

SEQ ID NO: 5 - DNA, 5916 bp, Artificial Sequence
  VC-LLL544-1qcz
ctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcnnnnnnnnnnn

SEQ ID NO: 6 - protein, 331 aa, Scenedesmus vacuolatus
MAPRRQSTLPQQTKAGSPTSGSDAAIPEPDVIDVWKAQYPLPDENVAGSMNEVKQLYRPPRNDVKGISIALGLIAAWCVLFYHGCWQIQLSGSQRSWWIDIAGTFILLEFVNTGLFITTHDAMHGTVCYRNRKLNDLLGRIAITLYAWFDYDMLHRKHWEHHNYTGQKGKDPDFHRGNPALPVWYARFMWEYSTPLQFAKIILVSQVLQALGVPYNNLCVYMAAAPLVAAFRLFYFGTYLPHLPPNAQEVMVWQKSHSSDAPSWLSFLKCYHFDYHWEHHRWPYAPWWELPKAKKITQQTQHHQQTKQQQPMQQAKAQVVSQLAPAGAVVE

SEQ ID NO: 7 - DNA, 1167 bp, Artificial Sequence
  synthetic fragment TP-RbcS/SV211 BKT
atggcctcaatgatctcaagttcagctgttactacagtctcaagagcaagtaccgttcaaagtgctgcagttgctccatttggtggacttaagagcatgactggttttccagtgaagaaagtgaacacagacattacgtccataacctcaaatggtggacgtgttaaatgtatggctccaagaaggcaatctacacttccacaacagactaaagctggttctcctacttcaggatcagatgcagctatacctgaaccagatgtcatagatgtatggaaggctcaatatcctcttcctgatgagaatgtagctggaagcatgaatgaggtcaagcagctttatagacctccaagaaatgacgtgaaaggcatctcgattgcattaggactcattgctgcatggtgtgtacttttctatcacggttgttggcagattcaactaagtgggagtcaaagatcatggtggatagacattgctggcacttttatcctcctggaatttgtgaataccggtttgttcatcacgactcatgatgcgatgcatggaacagtgtgttacaggaatcgtaagctcaatgatctactcggaagaattgccatcacactttacgcttggtttgattacgatatgcttcatcggaaacattgggagcaccataactatacaggacagaaaggaaaagacccagattttcacagaggtaatcctgctcttccagtatggtatgccagatttatgtgggagtattctactccactacaattcgcgaagattatactcgtttcgcaagttcttcaagctcttggtgttccctacaacaatctgtgcgtgtatatggcagctgctccacttgttgctgcatttcgactgttctattttggtacatacctaccacatcttcctccaaatgcacaagaagttatggtttggcagaagtctcattcctctgatgcaccttcttggctatccttcctcaaatgctaccactttgattaccattgggaacatcacagatggccttatgcaccatggtgggaacttcctaaggctaaaaagattacccagcaaacacagcatcatcaacagacgaaacagcaacaaccaatgcagcaagcaaaagctcaagtggtttctcaacttgcaccagcaggagctgttgtcgaatag

SEQ ID NO: 8 - DNA, 4476 bp, Artificial Sequence
  VC-SIW182-6
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtggtaccgggccccccctcgagtttaaaccactccgccctacactcgtatatatatgcctaaacctgccccgttcctcatatgtgatattattatttcattattaggtataagatagtaaacgataaggaaagacaatttattgagaaagccatgctaaaatatagatagatataccttagcaggtgtttattttacaacataacataacatagtagctagccagcaggcaggctaaaacatagtatagtctatctgcatgctcgagcggccgcctattcgacaacagctcctgctggtgcaagttgagaaaccacttgagcttttgcttgctgcattggttgttgctgtttcgtctgttgatgatgctgtgtttgctgggtaatctttttagccttaggaagttcccaccatggtgcataaggccatctgtgatgttcccaatggtaatcaaagtggtagcatttgaggaaggatagccaagaaggtgcatcagaggaatgagacttctgccaaaccataacttcttgtgcatttggaggaagatgtggtaggtatgtaccaaaatagaacagtcgaaatgcagcaacaagtggagcagctgccatatacacgcacagattgttgtagggaacaccaagagcttgaagaacttgcgaaacgagtataatcttcgcgaattgtagtggagtagaatactcccacataaatctggcataccatactggaagagcaggattacctctgtgaaaatctgggtcttttcctttctgtcctgtatagttatggtgctcccaatgtttccgatgaagcatatcgtaatcaaaccaagcgtaaagtgtgatggcaattcttccgagtagatcattgagcttacgattcctgtaacacactgttccatgcatcgcatcatgagtcgtgatgaacaaaccggtattcacaaattccaggaggataaaagtgccagcaatgtctatccaccatgatctttgactcccacttagttgaatctgccaacaaccgtgatagaaaagtacacaccatgcagcaatgagtcctaatgcaatcgagatgcctttcacgtcatttcttggaggtctataaagctgcttgacctcattcatgcttccagctacattctcatcaggaagaggatattgagccttccatacatctatgacatctggttcaggtatagctgcatctgatcctgaagtaggagaaccagctttagtctgttgtggaagtgtagattgccttcttggagccatacatttaacacgtccaccatttgaggttatggacgtaatgtctgtgttcactttcttcactggaaaaccagtcatgctcttaagtccaccaaatggagcaactgcagcactttgaacggtacttgctcttgagactgtagtaacagctgaacttgagatcattgaggccatttttctcacttctgtatgaattgcagatatctcgaagctagtgaacaagaaaaatgtacatacactgactactaatttatagccatcattactgctgatttggcaaatggcgtaatctcaaaccaaatcacgaggggtaataaatgttagccacataaattggtaagatcaaaggacgatattcagacatcttaagcatcgtggtgtgaggcgataattgttttgggctttttggctcttgtttatggcaagaaaatccaatttgacacatgctggctaattttcaaaaagcataaaggattagtcagggattctgaaactaggagtgcaattttacacacagaaaaatcacataatcgtaacctataatcacgatgtgattcaatgccatgattatatcttcaaactgaaatcatagagctcattaaattgttgtcttttgttatatattaatggtacataatgccctttaaatgcttttgtactttgcgaaattttgaaataaacaaaatacattgtttctgtatgcacatggaaagtactaataaacgaaaaagaaagtttctctgcaaaaaacaaaaaaaaatatatttagggatagacagaaatcattgtttaggaaagttgctctttattatctctactacttctccacgagaattaggcgccatgcaaatgacaccgatgctagaaacttcggcagtgtactattgtctggccgttttctgattatcgctcagtttgccaactactttggtcctttaacggtcgagatctgctaccatgcgataaatatcttgacagcgttttggaaagcgcgattgagaggaaacgtccaaactatttgtttattgggcttataattgactaaaaatgggccttgttaacagaatctcttgtattttttttcttttgaaatttacatcacttttccataacaaacgaaatgcaacaaactgtaaaaaaactttgtgttagatggagaatttacagccattcgattggtttcaaaatatgtcaagagtctcaatgatcggacggttgcaaagaggacacgagcctctgcagcgcaagggcgaattccagcacactggcggccgttactagtggatccgagctcggtaccaagcttggcgtaatcatggagcctgcttttttgtacaaacttgccatgattacgccaagcttgcatgccgtcgaccagatctgatatctgcggccgcctcgagcatatgctagaggatccccgggtacccaacttttctatacaaagttggatggcatgcaagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtc

SEQ ID NO: 9 - protein, 312 aa, Chlorella zofingiensis
MAPDVTHVQPRVQSPAGPDDEDDALSLWKAQYPMPEEKGTVSKPQAALKYRPPRSDWKGVSIACTVITLWTAVFYHGCWQIKLTGPDKSAWWDVVATFLALEFLNTGLFITTHDAMHGTIAIRNRRLNDLLGNIAISLYAWFDYDMLHKKHWEHHNFTGLPHKDPDFHRGDPALHKWFGRFMWEYATPLQFAKIFAYPFFLQSLRVQYPNLCVFLAAAPLVSAFRLFYFGTYLPHLPSNAQETMPWEKSHSADDPRPLSFLKCYHFDYHWEHHRWPYAPWWELPVCKRITKTLDAAVPGVQSDGTKKSQLVN

SEQ ID NO: 10 - DNA, 1110 bp, Artificial Sequence
  synthetic fragment TP-RbcS/CZ BKT
atggcctctatgatctcgtctagtgcagtgactacagtctcaagagcatccacagttcaatcagcagctgttgctccgtttggtggtctgaagtctatgactggatttcccgtgaagaaagtgaacacggatataacgtccattacctcgaatggaggtagagtgaagtgtatggcacctgatgtgacacatgttcagcctagagttcaaagtcctgctggtccagatgatgaggatgacgctttgtctctttggaaagcccaatatcctatgcctgaggaaaagggtacagtatcgaaaccacaagcagctctgaagtacagaccacctagatcagattggaaaggcgtttcaattgcctgtactgttatcacgctttggactgctgtgttctatcatggttgttggcaaatcaaactgactggaccagataagagtgcatggtgggacgttgttgcaactttcctagcgttggaatttctcaatactgggcttttcattaccactcatgatgctatgcatggaaccattgctatccgaaatcgcagactgaatgaccttcttgggaatattgcgatcagcttgtatgcttggttcgactacgatatgctgcacaagaaacattgggaacatcacaactttacaggacttccacacaaagatccagactttcacagaggagatcctgcactacacaaatggtttggcagatttatgtgggagtatgctacaccattgcaattcgcgaaaatcttcgcctatcccttctttctccaaagtctcagagtacagtacccaaacctatgcgtctttcttgcagctgcaccacttgttagcgcatttcgactcttttactttgggacatacttgcctcatttgccatctaatgcgcaagaaacaatgccttgggaaaagtcccattcagctgatgatccaagacctcttagcttcttgaaatgttatcactttgactatcattgggagcatcatagatggccttatgcaccttggtgggaacttcctgtatgcaaacggattaccaagacactagatgctgctgtaccaggtgtccaatcagatgggaccaaaaagagccaattggttaactag

SEQ ID NO: 11 - DNA, 4411 bp, Artificial Sequence
  VC-SIW198-1
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtggtaccgggccccccctcgagtttaaaccactccgccctacactcgtatatatatgcctaaacctgccccgttcctcatatgtgatattattatttcattattaggtataagatagtaaacgataaggaaagacaatttattgagaaagccatgctaaaatatagatagatataccttagcaggtgtttattttacaacataacataacatagtagctagccagcaggcaggctaaaacatagtatagtctatctgcatgcctagttaaccaattggctctttttggtcccatctgattggacacctggtacagcagcatctagtgtcttggtaatccgtttgcatacaggaagttcccaccaaggtgcataaggccatctatgatgctcccaatgatagtcaaagtgataacatttcaagaagctaagaggtcttggatcatcagctgaatgggacttttcccaaggcattgtttcttgcgcattagatggcaaatgaggcaagtatgtcccaaagtaaaagagtcgaaatgcgctaacaagtggtgcagctgcaagaaagacgcataggtttgggtactgtactctgagactttggagaaagaagggataggcgaagattttcgcgaattgcaatggtgtagcatactcccacataaatctgccaaaccatttgtgtagtgcaggatctcctctgtgaaagtctggatctttgtgtggaagtcctgtaaagttgtgatgttcccaatgtttcttgtgcagcatatcgtagtcgaaccaagcatacaagctgatcgcaatattcccaagaaggtcattcagtctgcgatttcggatagcaatggttccatgcatagcatcatgagtggtaatgaaaagcccagtattgagaaattccaacgctaggaaagttgcaacaacgtcccaccatgcactcttatctggtccagtcagtttgatttgccaacaaccatgatagaacacagcagtccaaagcgtgataacagtacaggcaattgaaacgcctttccaatctgatctaggtggtctgtacttcagagctgcttgtggtttcgatactgtacccttttcctcaggcataggatattgggctttccaaagagacaaagcgtcatcctcatcatctggaccagcaggactttgaactctaggctgaacatgtgtcacatcaggtgccatacacttcactctacctccattcgaggtaatggacgttatatccgtgttcactttcttcacgggaaatccagtcatagacttcagaccaccaaacggagcaacagctgctgattgaactgtggatgctcttgagactgtagtcactgcactagacgagatcatagaggccatttttctcacttctgtatgaattgcatctagatatctcgaagctagtgaacaagaaaaatgtacatacactgactactaatttatagccatcattactgctgatttggcaaatggcgtaatctcaaaccaaatcacgaggggtaataaatgttagccacataaattggtaagatcaaaggacgatattcagacatcttaagcatcgtggtgtgaggcgataattgttttgggctttttggctcttgtttatggcaagaaaatccaatttgacacatgctggctaattttcaaaaagcataaaggattagtcagggattctgaaactaggagtgcaattttacacacagaaaaatcacataatcgtaacctataatcacgatgtgattcaatgccatgattatatcttcaaactgaaatcatagagctcattaaattgttgtcttttgttatatattaatggtacataatgccctttaaatgcttttgtactttgcgaaattttgaaataaacaaaatacattgtttctgtatgcacatggaaagtactaataaacgaaaaagaaagtttctctgcaaaaaacaaaaaaaaatatatttagggatagacagaaatcattgtttaggaaagttgctctttattatctctactacttctccacgagaattaggcgccatgcaaatgacaccgatgctagaaacttcggcagtgtactattgtctggccgttttctgattatcgctcagtttgccaactactttggtcctttaacggtcgagatctgctaccatgcgataaatatcttgacagcgttttggaaagcgcgattgagaggaaacgtccaaactatttgtttattgggcttataattgactaaaaatgggccttgttaacagaatctcttgtattttttttcttttgaaatttacatcacttttccataacaaacgaaatgcaacaaactgtaaaaaaactttgtgttagatggagaatttacagccattcgattggtttcaaaatatgtcaagagtctcaatgatcggacggttgcaaagaggacacgagcctctgcagcgcaagggcgaattccagcacactggcggccgttactagtggatccgagctcggtaccaagcttggcgtaatcatggagcctgcttttttgtacaaacttgccatgattacgccaagcttgcatgccgtcgaccagatctgatatctgcggccgcctcgagcatatgctagaggatccccgggtacccaacttttctatacaaagttggatggcatgcaagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtc

SEQ ID NO: 12 - protein, 328 aa, Chlamydomonas reinhardtii
MGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALA

SEQ ID NO: 13 - DNA, 1158 bp, Artificial Sequence
  synthetic fragment TP-RbcS/CR BKT
atggcgagtatgatctcatctagtgcagttacaaccgtttcaagagcttcaactgtgcaatctgctgcagtagctccatttggtggcttgaaaagcatgacaggttttcctgtcaagaaagttaacaccgacattacgagcattacgtctaatggagggagagtcaaatgtatgggaccaggaatacagccaacaagtgctcgtccttgttctaggactaagcactctaggtttgcccttttagctgcagctctaactgctaggagagttaagcagtttactaagcaattccggtcaagaagaatggctgaggatatcctcaaactgtggcagagacaatatcaccttcctagagaagattccgataaacgaaccttaagggaaagagtacacctctatagaccacctagaagtgatctaggtggaattgctgttgctgttacagtgattgctctttgggctactttgttcgtttacggtctttggtttgtaaaacttccctgggcacttaaagttggggaaacagcaacttcttgggcaacaatagctgcagttttttttagccttgagttcctttatactggcctcttcattacaacccatgatgcaatgcatggaacaatcgcactgagaaatcgcagattgaacgactttcttggtcaacttgccatctcactttacgcatggtttgattactcggttctgcatcgaaagcattgggaacatcacaatcatactggagaaccaagagttgacccagattttcacagaggaaacccaaatctagctgtttggtttgcgcaattcatggtctcatacatgactctaagccagtttctcaagattgctgtttggtcgaatcttctccttcttgctggtgcacctctggctaaccaactcttgtttatgacagctgcaccaatactttcggcattcagactcttctattacggtacgtatgtacctcatcatccagaaaagggtcatacaggagctatgccatggcaagtttcaaggacgagttcagcttcaagactccagtcctttctaacctgctatcacttcgatctacattgggaacatcatcgatggccttatgctccttggtgggaactgcctaaatgcagacaaattgctcgcggtgcagctttagcttag

SEQ ID NO: 14 - DNA, 4471 bp, Artificial Sequence
  VC-SIW195-1
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtggtaccgggccccccctcgagtttaaaccactccgccctacactcgtatatatatgcctaaacctgccccgttcctcatatgtgatattattatttcattattaggtataagatagtaaacgataaggaaagacaatttattgagaaagccatgctaaaatatagatagatataccttagcaggtgtttattttacaacataacataacatagtagctagccagcaggcaggctaaaacatagtatagtctatctgcatgctcgagcggccgcctaagctaaagctgcaccgcgagcaatttgtctgcatttaggcagttcccaccaaggagcataaggccatcgatgatgttcccaatgtagatcgaagtgatagcaggttagaaaggactggagtcttgaagctgaactcgtccttgaaacttgccatggcatagctcctgtatgacccttttctggatgatgaggtacatacgtaccgtaatagaagagtctgaatgccgaaagtattggtgcagctgtcataaacaagagttggttagccagaggtgcaccagcaagaaggagaagattcgaccaaacagcaatcttgagaaactggcttagagtcatgtatgagaccatgaattgcgcaaaccaaacagctagatttgggtttcctctgtgaaaatctgggtcaactcttggttctccagtatgattgtgatgttcccaatgctttcgatgcagaaccgagtaatcaaaccatgcgtaaagtgagatggcaagttgaccaagaaagtcgttcaatctgcgatttctcagtgcgattgttccatgcattgcatcatgggttgtaatgaagaggccagtataaaggaactcaaggctaaaaaaaactgcagctattgttgcccaagaagttgctgtttccccaactttaagtgcccagggaagttttacaaaccaaagaccgtaaacgaacaaagtagcccaaagagcaatcactgtaacagcaacagcaattccacctagatcacttctaggtggtctatagaggtgtactctttcccttaaggttcgtttatcggaatcttctctaggaaggtgatattgtctctgccacagtttgaggatatcctcagccattcttcttgaccggaattgcttagtaaactgcttaactctcctagcagttagagctgcagctaaaagggcaaacctagagtgcttagtcctagaacaaggacgagcacttgttggctgtattcctggtcccatacatttgactctccctccattagacgtaatgctcgtaatgtcggtgttaactttcttgacaggaaaacctgtcatgcttttcaagccaccaaatggagctactgcagcagattgcacagttgaagctcttgaaacggttgtaactgcactagatgagatcatactcgccatttttctcacttctgtatgaattgcatctagatatctcgaagctagtgaacaagaaaaatgtacatacactgactactaatttatagccatcattactgctgatttggcaaatggcgtaatctcaaaccaaatcacgaggggtaataaatgttagccacataaattggtaagatcaaaggacgatattcagacatcttaagcatcgtggtgtgaggcgataattgttttgggctttttggctcttgtttatggcaagaaaatccaatttgacacatgctggctaattttcaaaaagcataaaggattagtcagggattctgaaactaggagtgcaattttacacacagaaaaatcacataatcgtaacctataatcacgatgtgattcaatgccatgattatatcttcaaactgaaatcatagagctcattaaattgttgtcttttgttatatattaatggtacataatgccctttaaatgcttttgtactttgcgaaattttgaaataaacaaaatacattgtttctgtatgcacatggaaagtactaataaacgaaaaagaaagtttctctgcaaaaaacaaaaaaaaatatatttagggatagacagaaatcattgtttaggaaagttgctctttattatctctactacttctccacgagaattaggcgccatgcaaatgacaccgatgctagaaacttcggcagtgtactattgtctggccgttttctgattatcgctcagtttgccaactactttggtcctttaacggtcgagatctgctaccatgcgataaatatcttgacagcgttttggaaagcgcgattgagaggaaacgtccaaactatttgtttattgggcttataattgactaaaaatgggccttgttaacagaatctcttgtattttttttcttttgaaatttacatcacttttccataacaaacgaaatgcaacaaactgtaaaaaaactttgtgttagatggagaatttacagccattcgattggtttcaaaatatgtcaagagtctcaatgatcggacggttgcaaagaggacacgagcctctgcagcgcaagggcgaattccagcacactggcggccgttactagtggatccgagctcggtaccaagcttggcgtaatcatggagcctgcttttttgtacaaacttgccatgattacgccaagcttgcatgccgtcgaccagatctgatatctgcggccgcctcgagcatatgctagaggatccccgggtacccaacttttctatacaaagttggatggcatgcaagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtc

SEQ ID NO: 15 - DNA, 1515 bp, Lycopersicon esculentum
  CDS (7)..(1500)
cccgggatggaagctcttctcaagccttttccatctcttttactttcctctcctacaccctataggtctattgtccaacaaaatccttcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcgaatcgggctcaattcgacgtgatcattatcggagctggccctgctgggctcaggctagctgaacaagtttctaaatatggtattaaggtatgttgtgttgacccttcaccactctccatgtggccaaataattatggtgtttgggttgatgagtttgagaatttaggactggaagattgtttagatcataaatggcctatgacttgtgtgcatataaatgataacaaaactaagtatttgggaagaccatatggtagagttagtagaaagaagctgaagttgaaattgttgaatagttgtgttgagaacagagtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaggccaagaaaccatggttatcaaattgctcatggggttttagtagaagttgataatcatccatttgatttggataaaatggtgcttatggattggagggattctcatttgggtaatgagccatatttaagggtgaataatgctaaagaaccaacattcttgtatgcaatgccatttgatagagatttggttttcttggaagagacttctttggtgagtcgtcctgttttatcgtatatggaagtaaaaagaaggatggtggcaagattaaggcatttggggatcaaagtgaaaagtgttattgaggaagagaaatgtgtgatccctatgggaggaccacttccgcggattcctcaaaatgttatggctattggtgggaattcagggatagttcatccatcaacagggtacatggtggctaggagcatggctttagcaccagtactagctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgacgctttctttgatcttgatcctaaatactggcaagggttcctttcttcaagattgtctgtcaaagaacttggtttactcagcttgtgtcttttcggacatggctcaaacatgactaggttggatattgttacaaaatgtcctcttcctttggttagactgattggcaatctagcaatagagagcctttgaatgtgactgcag

SEQ ID NO: 16 - protein, 498 aa, Lycopersicon esculentum
MEALLKPFPSLLLSSPTPYRSIVQQNPSFLSPTTKKKSRKCLLRNKSSKLFCSFLDLAPTSKPESLDVNISWVDPNSNRAQFDVIIIGAGPAGLRLAEQVSKYGIKVCCVDPSPLSMWPNNYGVWVDEFENLGLEDCLDHKWPMTCVHINDNKTKYLGRPYGRVSRKKLKLKLLNSCVENRVKFYKAKVWKVEHEEFESSIVCDDGKKIRGSLVVDASGFASDFIEYDRPRNHGYQIAHGVLVEVDNHPFDLDKMVLMDWRDSHLGNEPYLRVNNAKEPTFLYAMPFDRDLVFLEETSLVSRPVLSYMEVKRRMVARLRHLGIKVKSVIEEEKCVIPMGGPLPRIPQNVMAIGGNSGIVHPSTGYMVARSMALAPVLAEAIVEGLGSTRMIRGSQLYHRVWNGLWPLDRRCVRECYSFGMETLLKLDLKGTRRLFDAFFDLDPKYWQGFLSSRLSVKELGLLSLCLFGHGSNMTRLDIVTKCPLPLVRLIGNLAIESL

SEQ ID NO: 17 - DNA, 1515 bp, Artificial sequence
  CDS (7)..(1500)
  beta-cyclase coding sequence
cccgggatggaagcactactgaaaccctttccttcattgctactctcatcacctactccgtatagatcaatcgtgcaacaaaacccttcctttcttagtcctacaacgaaaaagaaaagtcgtaaatgtctactcaggaataaaagcagtaagttgttctgttcgttcttggatcttgctccaacttctaagccagaatcacttgacgttaatatttcatgggttgatccaaactcaaacagagctcaatttgacgtgataattattggtgctggacctgctggtttgagattggcagagcaggtgtcgaaatacgggattaaagtgtgctgtgttgatccttcaccattgtctatgtggcctaataattatggcgtatgggttgatgagttcgagaatttggggttggaagattgtttggaccataaatggcctatgacttgtgtccacattaatgacaacaagaccaaatacttgggcagaccttatgggagagtttccagaaagaaactaaagctgaaattgttgaactcgtgtgttgaaaacagagtcaagttttacaaagctaaggtttggaaagttgaacacgaagagtttgagtcttcaatcgtttgtgacgacggtaagaaaatccgtggatcattggtggtcgatgcttcaggatttgcttcagactttatcgagtatgatagacctagaaaccatgggtaccaaattgcccatggtgttttggtagaagttgataatcacccttttgatctcgacaagatggtcttgatggattggcgcgatagtcacctaggaaatgagccttacttgagagttaacaacgctaaagaacccacttttctctatgcaatgccattcgatagagacttggttttcctagaagaaacaagcttagtttcaagaccagtactctcttatatggaggtaaaacggagaatggtggcaagattgagacatttggggattaaagtgaaatccgttattgaggaagagaagtgcgtaattcctatgggaggtcctttgccaagaattccacagaacgttatggctattggtggaaattcagggattgttcatcctagtacaggctatatggtcgccagatcaatggctcttgctccagttcttgctgaagcaatcgttgaaggacttggttctacacgtatgatcagaggatcacaactctatcacagagtctggaacggcttgtggccattggatagacgttgtgttagagagtgctatagctttggtatggaaactttgctgaagttggacttgaaaggtacaaggagattgtttgacgcgttctttgacttggatccaaagtattggcaaggatttctgtcctctaggttgtctgtaaaggaacttggtttgttgtcattgtgtctctttggtcatggttcgaatatgacacgcttggatattgttacgaagtgtccattgccattggttcgattgattggtaacttggctattgagtccttgtgaatgtgactgcag

SEQ ID NO: 18 - DNA, 23 bp, Artificial sequence
  Primer
cccgggatggaagctcttctcaa

SEQ ID NO: 19 - DNA, 28 bp, Artificial sequence
  Primer
ctgcagtcacattcaaaggctctctatt

SEQ ID NO: 20 - DNA, 1010 bp, Scenedesmus vacuolatus
  CDS (7)..(1002)
gcgcatatggctcccaggcggcaatcaacgctgccgcagcagaccaaagctggctctccaaccagtggctcagatgctgccatccctgagcccgatgtcatcgacgtgtggaaagcgcaataccctctgccggatgaaaatgtagcagggagcatgaatgaggtcaagcagttgtacaggccacctcgcaatgatgtgaagggcataagcattgccttgggcctgattgcagcctggtgcgtgctgttttaccacggctgctggcagatccagctgtctggcagtcagcgctcctggtggattgacattgctggcacatttattttgttggagttcgtcaacacaggccttttcatcaccacgcacgatgccatgcatggcactgtttgttacaggaaccgcaagctgaacgatctgttgggtcgtatagccatcacactgtacgcctggtttgactacgacatgcttcacaggaagcactgggagcatcacaactacacaggacagaagggcaaagaccctgacttccacaggggcaaccctgcactgccagtgtggtatgccaggttcatgtgggaatactccacccccttgcagtttgccaaaatcatcctggtgagtcaggtgctgcaagccctgggagtgccctacaacaacctgtgtgtctacatggctgctgcgcccctggtggccgccttcaggctgttctattttggcacctacctgccgcacttgccccccaacgcccaggaggtgatggtgtggcagaagagtcactctagtgatgctccctcctggctgtctttcctcaagtgttaccactttgattatcattgggaacaccacagatggccatatgctccctggtgggagttgccgaaggcgaagaaaattacacaacaaactcagcatcaccaacaaaccaagcagcagcagcccatgcagcaggcaaaagcgcaggttgtctcccagctggcccctgcaggagcagtagtggagtaagtcgaccg

SEQ ID NO: 21 - DNA, 24 bp, Artificial sequence
  Primer
gcgcatatggctcccaggcggcaa

SEQ ID NO: 22 - DNA, 26 bp, Artificial sequence
  Primer
cggtcgacttactccactactgctcc

SEQ ID NO: 23 - protein, 331 aa, Scenedesmus vacuolatus; identical to SEQ ID NO: 6

SEQ ID NO: 24 - DNA, 5916 bp, Artificial sequence
  VC-LLL544-1qcz, base vector used for cloning of overexpression constructs
gattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatggaacgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtggtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagnnnnnnnnnnn

SEQ ID NO: 25 - DNA, 7878 bp, Artificial sequence
  T-DNA of binary vector with expression cassette
gacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaagcttgcatgccatccaactttgtatagaaaagttgccatgattacgccaagcttgcatgccgtcgaggcttaacgagcattttctaagaaacggatacaattttctatgaaacacaccaagaaaaacattagcatatagttgagttttaagttattattttgtagaaacatatctcatatctgcaaactttcattgtatttcgaataaatctcaatttattttgatttttttatcgaaacacatatgtcaaaaggccataactttattaatattttagaactgatgttcttgactacagcaattctaaatttattttataattattttattacagaaagtggtcacctcaacattctattgcaggaattgtcgaccagatctgatatctgcggccgcctattcgacaacagctcctgctggtgcaagttgagaaaccacttgagcttttgcttgctgcattggttgttgctgtttcgtctgttgatgatgctgtgtttgctgggtaatctttttagccttaggaagttcccaccatggtgcataaggccatctgtgatgttcccaatggtaatcaaagtggtagcatttgaggaaggatagccaagaaggtgcatcagaggaatgagacttctgccaaaccataacttcttgtgcatttggaggaagatgtggtaggtatgtaccaaaatagaacagtcgaaatgcagcaacaagtggagcagctgccatatacacgcacagattgttgtagggaacaccaagagcttgaagaacttgcgaaacgagtataatcttcgcgaattgtagtggagtagaatactcccacataaatctggcataccatactggaagagcaggattacctctgtgaaaatctgggtcttttcctttctgtcctgtatagttatggtgctcccaatgtttccgatgaagcatatcgtaatcaaaccaagcgtaaagtgtgatggcaattcttccgagtagatcattgagcttacgattcctgtaacacactgttccatgcatcgcatcatgagtcgtgatgaacaaaccggtattcacaaattccaggaggataaaagtgccagcaatgtctatccaccatgatctttgactcccacttagttgaatctgccaacaaccgtgatagaaaagtacacaccatgcagcaatgagtcctaatgcaatcgagatgcctttcacgtcatttcttggaggtctataaagctgcttgacctcattcatgcttccagctacattctcatcaggaagaggatattgagccttccatacatctatgacatctggttcaggtatagctgcatctgatcctgaagtaggagaaccagctttagtctgttgtggaagtgtagattgccttcttggagccatacatttaacacgtccaccatttgaggttatggacgtaatgtctgtgttcactttcttcactggaaaaccagtcatgctcttaagtccaccaaatggagcaactgcagcactttgaacggtacttgctcttgagactgtagtaacagctgaacttgagatcattgaggccatttttctcacttctgtatgaattgcagatatctcgaagctagtgaacaagaaaaatgtacatacactgactactaatttatagccatcattactgctgatttggcaaatggcgtaatctcaaaccaaatcacgaggggtaataaatgttagccacataaattggtaagatcaaaggacgatattcagacatcttaagcatcgtggtgtgaggcgataattgttttgggctttttggctcttgtttatggcaagaaaatccaatttgacacatgctggctaattttcaaaaagcataaaggattagtcagggattctgaaactaggagtgcaattttacacacagaaaaatcacataatcgtaacctataatcacgatgtgattcaatgccatgattatatcttcaaactgaaatcatagagctcattaaattgttgtcttttgttatatattaatggtacataatgccctttaaatgcttttgtactttgcgaaattttgaaataaacaaaatacattgtttctgtatgcacatggaaagtactaataaacgaaaaagaaagtttctctgcaaaaaacaaaaaaaaatatatttagggatagacagaaatcattgtttaggaaagttgctctttattatctctactacttctccacgagaattaggcgccatgcaaatgacaccgatgctagaaacttcggcagtgtactattgtctggccgttttctgattatcgctcagtttgccaactactttggtcctttaacggtcgagatctgctaccatgcgataaatatcttgacagcgttttggaaagcgcgattgagaggaaacgtccaaactatttgtttattgggcttataattgactaaaaatgggccttgttaacagaatctcttgtattttttttcttttgaaatttacatcacttttccataacaaacgaaatgcaacaaactgtaaaaaaactttgtgttagatggagaatttacagccattcgattggtttcaaaatatgtcaagagtctcaatgatcggacggttgcaaagaggacacgagcctctgcagcgcaagggcgaattccagcacactggcggccgttactagtggatccgagctcggtacccaagtttgtacaaaaaagcaggctggtacccggggatcctctagcatatgctcgaggcggccgcagatatcagatctggtcgacggcatgcaagcttggctaatcatggacccagctttcttgtacaaagtggggtaccactttcgtaatcatattacccaaccgcttaccttaatacagtgtctagctattaagcaaaatataaaataaaacaatcaaaactaaaaaaaatatataaaataaaaactagtttttctcgaagacctacttatctgaattatcagtagtattaatctggcgctgtctgcatttagtacatacagcagccgtcacactcaactcatgaaagagtttcgtttttcatttaatagtagtggtaacttaaaccataagtaattagcatcaatagtcatttcgtccataaaagtaataagttatatctgttaaaaagaaatatttaaacaatacatttagcggatagcggagaaatatgactccagtgacaaagagggtctttactataaaggtaaaattgcggcaaactctccaatcaattgcgttttttgtaatttacttcttaatatatgaaaatgtgcgatatgcgtcctaatctattaatttttgtatgatatattctctgagtcgttgatcaatcattcattttggacacgtgtctgagtttattcattagatttaacatcagaggtaaatcgcacaaaaattaatagattatggggcatatcgcacaatttcatctgttagggaacacaataaaagcacccatagattaaagaggccccatagattaaagagtttgccgcaattttctctactataaatatattgttggtgataattcaaacacaagaagcctaatataaattcagcaagcatcttttctagctgatatccagatgaaataacttcacatttctatcacaattacatgatatatatctctatccatgtcttttttaataaaataacaaaaaggaaagaaaaaagttaacccctcacttttctatcgtgttgcacttgcgctcccaacttttaaattttgcatttgtctttcaacctttcgtagtaagccacattgctattcacgccatctgagaattgagtaaaaagcttcggtagcaaaatcgagccaatgaaatatttgttggcaaagtgcaaaaagggtcaaggttgggggtaactaagtgccctttttgttttcctttaaatcaaaacgtataataaaacattggattttgaaatatttttcttcgtatattgcaccctaattttttgatatcacatccaatattatcctctctttgttattatttaaaaaacattagtattatcataattgtcaaataattgcaagaagagaaaaaaaatgtccattgtcaccgcaaagactttctgaaacttcgaactacttctcataaatacaagaattaatagctacccgcttgaaagctaaccaactaacacatatcaaacttcattgatcgcatttaatcctctcgccaacccaccaccctatcaaccatcactacaataaatacaccaccccttcatctcaatcctcaaaccaaacaacggatcctctagcatatgctcgagcggccgccagtgtgatggatatctgcagaattcgcccttcccgggatggaagctcttctcaagccttttccatctcttttactttcctctcctacaccctataggtctattgtccaacaaaatccttcttttctaagtcccaccaccaaaaaaaaatcaagaaaatgtcttcttagaaacaaaagtagtaaacttttttgtagctttcttgatttagcacccacatcaaagccagagtctttagatgttaacatctcatgggttgatcctaattcgaatcgggctcaattcgacgtgatcattatcggagctggccctgctgggctcaggctagctgaacaagtttctaaatatggtattaaggtatgttgtgttgacccttcaccactctccatgtggccaaataattatggtgtttgggttgatgagtttgagaatttaggactggaagattgtttagatcataaatggcctatgacttgtgtgcatataaatgataacaaaactaagtatttgggaagaccatatggtagagttagtagaaagaagctgaagttgaaattgttgaatagttgtgttgagaacagagtgaagttttataaagctaaggtttggaaagtggaacatgaagaatttgagtcttcaattgtttgtgatgatggtaagaagataagaggtagtttggttgtggatgcaagtggttttgctagtgattttatagagtatgacaggccaagaaaccatggttatcaaattgctcatggggttttagtagaagttgataatcatccatttgatttggataaaatggtgcttatggattggagggattctcatttgggtaatgagccatatttaagggtgaataatgctaaagaaccaacattcttgtatgcaatgccatttgatagagatttggttttcttggaagagacttctttggtgagtcgtcctgttttatcgtatatggaagtaaaaagaaggatggtggcaagattaaggcatttggggatcaaagtgaaaagtgttattgaggaagagaaatgtgtgatccctatgggaggaccacttccgcggattcctcaaaatgttatggctattggtgggaattcagggatagttcatccatcaacagggtacatggtggctaggagcatggctttagcaccagtactagctgaagccatcgtcgaggggcttggctcaacaagaatgataagagggtctcaactttaccatagagtttggaatggtttgtggcctttggatagaagatgtgttagagaatgttattcatttgggatggagacattgttgaagcttgatttgaaagggactaggagattgtttgacgctttctttgatcttgatcctaaatactggcaagggttcctttcttcaagattgtctgtcaaagaacttggtttactcagcttgtgtcttttcggacatggctcaaacatgactaggttggatattgttacaaaatgtcctcttcctttggttagactgattggcaatctagcaatagagagcctttgaatgtgactgcagaagggcgaattccagcacactggcggccgttactagtggatctggtcgacaattcctgcaatagaatgttgaggtgaccactttctgtaataaaataattataaaataaatttagaattgctgtagtcaagaacatcagttctaaaatattaataaagttatggccttttgacatatgtgtttcgataaaaaaatcaaaataaattgagatttattcgaaatacaatgaaagtttgcagatatgagatatgtttctacaaaataataacttaaaactcaactatatgctaatgtttttcttggtgtgtttcatagaaaattgtatccgtttcttagaaaatgctcgttaagcctcgacggcatgcaagcttggcgtaatcatggcaactttattatacaaagttggataattcactggccgtcgttttacaacgactcaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatcggtcgatcgactcagatctgggtaactggcctaactggccttggaggagctggcaactcaaaatccctttgccaaaaaccaacatcatgccatccaccatgcttgtatccagccgcgcgcaatgtaccccgcgctgtgtatcccaaagcctcatgcaacctaacagatggatcgtttggaaggcctataacagcaaccacagacttaaaaccttgcgcctccatagacttaagcaaatgtgtgtacaatgtagatcctaggcccaacctttgatgcctatgtgacacgtaaacagtactctcaactgtccaatcgtaagcgttcctagccttccagggcccagcgtaagcaataccagccacaacaccctcaacctcagcaaccaaccaagggtatctatcttgcaacctctctaggtcatcaatccactcttgtggtgtttgtggctctgtcctaaagttcactgtagacgtctcaatgtaatggttaacgatgtcacaaaccgcggccatatcggctgctgtagctggcctaatctcaactggtctcctctccggagacatgtcgagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcac

SEQ ID NO: 26 - DNA, 34 bp, Artificial sequence
  Primer
ctggtaccactttcgtaatcatattacccaaccg

SEQ ID NO: 27 - DNA, 34 bp, Artificial sequence
  Primer
ctggatccgttgtttggtttgaggattgagatga

SEQ ID NO: 28 - DNA, 1452 bp, Antirrhinum majus
actttcgtaatcatattacccaaccgcttaccttaatacagtgtctagctattaagcaaaatataaaataaaacaatcaaaactaaaaaaaatatataaaataaaaactagtttttctcgaagacctacttatctgaattatcagtagtattaatctggcgctgtctgcatttagtacatacagcagccgtcacactcaactcatgaaagagtttcgtttttcatttaatagtagtggtaacttaaaccataagtaattagcatcaatagtcatttcgtccataaaagtaataagttatatctgttaaaaagaaatatttaaacaatacatttagcggatagcggagaaatatgactccagtgacaaagagggtctttactataaaggtaaaattgcggcaaactctccaatcaattgcgttttttgtaatttacttcttaatatatgaaaatgtgcgatatgcgtcctaatctattaatttttgtatgatatattctctgagtcgttgatcaatcattcattttggacacgtgtctgagtttattcattagatttaacatcagaggtaaatcgcacaaaaattaatagattatggggcatatcgcacaatttcatctgttagggaacacaataaaagcacccatagattaaagaggccccatagattaaagagtttgccgcaattttctctactataaatatattgttggtgataattcaaacacaagaagcctaatataaattcagcaagcatcttttctagctgatatccagatgaaataacttcacatttctatcacaattacatgatatatatctctatccatgtcttttttaataaaataacaaaaaggaaagaaaaaagttaacccctcacttttctatcgtgttgcacttgcgctcccaacttttaaattttgcatttgtctttcaacctttcgtagtaagccacattgctattcacgccatctgagaattgagtaaaaagcttcggtagcaaaatcgagccaatgaaatatttgttggcaaagtgcaaaaagggtcaaggttgggggtaactaagtgccctttttgttttcctttaaatcaaaacgtataataaaacattggattttgaaatatttttcttcgtatattgcaccctaattttttgatatcacatccaatattatcctctctttgttattatttaaaaaacattagtattatcataattgtcaaataattgcaagaagagaaaaaaaatgtccattgtcaccgcaaagactttctgaaacttcgaactacttctcataaatacaagaattaatagctacccgcttgaaagctaaccaactaacacatatcaaacttcattgatcgcatttaatcctctcgccaacccaccaccctatcaaccatcactacaataaatacaccaccccttcatctcaatcctcaaaccaaacaac

SEQ ID NO: 29 - DNA, 4903 bp, Artificial sequence
  VC-SBT477
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtccatgattagccaagcttgcatgccgtcgaccagatctgatatctgcggccgcctcgagcatatgctagaggatccccgggtaccagcctgcttttttgtacaaacttgccatgattacgccaagcttgcatgccgtcgaccagatctgatatctgcggccgcgaattcgcccttctggtaccactttcgtaatcatattacccaaccgcttaccttaatacagtgtctagctattaagcaaaatataaaataaaacaatcaaaactaaaaaaaatatataaaataaaaactagtttttctcgaagacctacttatctgaattatcagtagtattaatctggcgctgtctgcatttagtacatacagcagccgtcacactcaactcatgaaagagtttcgtttttcatttaatagtagtggtaacttaaaccataagtaattagcatcaatagtcatttcgtccataaaagtaataagttatatctgttaaaaagaaatatttaaacaatacatttagcggatagcggagaaatatgactccagtgacaaagagggtctttactataaaggtaaaattgcggcgaactctccaatcaattgcgttttttgtaatttacttcttaatatatgaaaatgtgcgatatgcgtcctaatctattaatttttgtatgatatattctctgagtcgttgatcaatcattcattttggacacgtgtctgagtttattcattagatttaacatcagaggtaaatcgcacaaaaattaatagattatggggcatatcgcacaatttcatctgttagggaacacaataaaagcacccatagattaaagaggccccatagattaaagagtttgccgcaattttctctactataaatatattgttggtgataattcaaacacaagaagcctaatataaattcagcaagcatcttttctagctgatatccagatgaaataacttcacatttctatcacaattacatgatatatatctctatccatgtcttttttaataaaataacaaaaaggaaagaaaaaagttaacccctcacttttctatcgtgttgcacttgcgctcccaacttttaaattttgcatttgtctttcaacctttcgtagtaagccacattgctattcacgccatctgagaattgagtaaaaagcttcggtagcaaaatcgagccaatgaaatatttgttggcaaagtgcaaaaagggtcaaggttgggggtaactaagtgccctttttgttttcctttaaatcaaaacgtataataaaacattggattttgaaatatttttcttcgtatattgcaccctaattttttgatatcacatccaatattatcctctctttgttattatttaaaaaacattagtattatcataattgtcaaataattgcaagaagagaaaaaaaatgtccattgtcaccgcaaagactttctgaaacttcgaactacttctcataaatacaagaattaatagctacccgcttgaaagctaaccaactaacacatatcaaacttcattgatcgcatttaatcctctcgccaacccaccaccctatcaaccatcactacaataaatacaccaccccttcatctcaatcctcaaaccaaacaacggatccaagctttgcaattcatacagaagtgagaaaaatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatggctcccaggcggcaatcaacgctgccgcagcagaccaaagctggctctccaaccagtggctcagatgctgccatccctgagcccgatgtcatcgacgtgtggaaagcgcaataccctctgccggatgaaaatgtagcagggagcatgaatgaggtcaagcagttgtacaggccacctcgcaatgatgtgaagggcataagcattgccttgggcctgattgcagcctggtgcgtgctgttttaccacggctgctggcagatccagctgtctggcagtcagcgctcctggtggattgacattgctggcacatttattttgttggagttcgtcaacacaggccttttcatcaccacgcacgatgccatgcatggcactgtttgttacaggaaccgcaagctgaacgatctgttgggtcgtatagccatcacactgtacgcctggtttgactacgacatgcttcacaggaagcactgggagcatcacaactacacaggacagaagggcaaagaccctgacttccacaggggcaaccctgcactgccagtgtggtatgccaggttcatgtgggaatactccacccccttgcagtttgccaaaatcatcctggtgagtcaggtgctgcaagccctgggagtgccctacaacaacctgtgtgtctacatggctgctgcgcccctggtggccgccttcaggctgttctattttggcacctacctgccgcacttgccccccaacgcccaggaggtgatggtgtggcagaagagtcactctagtgatgctccctcctggctgtctttcctcaagtgttaccactttgattatcattgggaacaccacagatggccatatgctccctggtgggagttgccgaaggcgaagaaaattacacaacaaactcagcatcaccaacaaaccaagcagcagcagcccatgcagcaggcaaaagcgcaggttgtctcccagctggcccctgcaggagcagtagtggagtaagtcgaccgaagggcgaattctagactatactatgttttagcctgcctgctggctagctactatgttatgttatgttgtaaaataaacacctgctaaggtatatctatctatattttagcatggctttctcaataaattgtctttccttatcgtttactatcttatacctaataatgaaataataatatcacatatgaggaacggggcaggtttaggcatatatatacgagtgtagggcggagtggtttaaactcgagcatatgctagaggatccccgggtacccaacttttctatacaaagttggatggcatgcaagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtc

SEQ ID NO: 30 - DNA, 996 bp, Scenedesmus vacuolatus
  CDS (1)..(993)
atggctcccaggcggcaatcaacgctgccgcagcagaccaaagctggctctccaaccagtggctcagatgctgccatccctgagcccgatgtcatcgacgtgtggaaagcgcaataccctctgccggatgaaaatgtagcagggagcatgaatgaggtcaagcagttgtacaggccacctcgcaatgatgtgaagggcataagcattgccttgggcctgattgcagcctggtgcgtgctgttttaccacggctgctggcagatccagctgtctggcagtcagcgctcctggtggattgacattgctggcacatttattttgttggagttcgtcaacacaggccttttcatcaccacgcacgatgccatgcatggcactgtttgttacaggaaccgcaagctgaacgatctgttgggtcgtatagccatcacactgtacgcctggtttgactacgacatgcttcacaggaagcactgggagcatcacaactacacaggacagaagggcaaagaccctgacttccacaggggcaaccctgcactgccagtgtggtatgccaggttcatgtgggaatactccacccccttgcagtttgccaaaatcatcctggtgagtcaggtgctgcaagccctgggagtgccctacaacaacctgtgtgtctacatggctgctgcgcccctggtggccgccttcaggctgttctattttggcacctacctgccgcacttgccccccaacgcccaggaggtgatggtgtggcagaagagtcactctagtgatgctccctcctggctgtctttcctcaagtgttaccactttgattatcattgggaacaccacagatggccatatgctccctggtgggagttgccgaaggcgaagaaaattacacaacaaactcagcatcaccaacaaaccaagcagcagcagcccatgcagcaggcaaaagcgcaggttgtctcccagctggcccctgcaggagcagtagtggagtaa

SEQ ID NO: 31 - protein, 331 aa, Scenedesmus vacuolatus; identical to SEQ ID NO: 6

(19) United States
(12) Patent Application Publication
(10) Pub. No.: US 2012/0156718 A1
(43) Pub. Date: Jun. 21, 2012

(54) Production of Ketocarotenoids in Plants

(75) Inventors: Ralf Flachmann, Mutterstadt (DE); Irina Wenderoth, Mutterstadt (DE); Christel Renate Schopter, Ludwigshafen (DE); Bettina Tschiersch, Quedlinburg (DE); Hannia Bridg-Giannakopoulos, Quedlinburg (DE); Michael Leps, Halberstadt (DE); Ute Linemann, Gatersleben (DE); George Mather Sauer, Quedlinburg (DE)

(73) Assignee: BASF Plant Science GmbH, Ludwigshafen (DE)

(21) Appl. No.: 13/140,218

(22) PCT Filed: Dec. 8, 2009
(86) PCT No.: PCT/EP09/66609

§ 371 Date: Jun. 16, 2011

Related U.S. Application Data
(60) Provisional application No. 61/143,828, filed on Jan. 12, 2009.
(60) Provisional application No. 61/162,354, filed on Mar. 23, 2009.

(30) Foreign Application Priority Data
Dec. 17, 2008 (EP) 08171888.4
Mar. 6, 2009 (EP) 09154487.4
Mar. 20, 2009 (EP) 09155754.6

(51) Int. Cl. C12P 23/00; C12N 1/00; C12N 15/53; A01H 5/10; A01H 1/06; C12N 15/63; A01H 5/00

(52) U.S. Cl. 435/67; 435/320.1; 435/243; 800/298; 800/320; 800/306; 800/317; 800/278; 536/23.2; 800/287

(57) ABSTRACT

The present invention relates to optimized ketolase coding sequences, corresponding coding sequences and genetic constructs alone or in combination with beta-cyclase coding sequences, their use for the expression in plants, in particular in plants of the genus Tagetes, to such genetically modified plants, to a process for the preparation of carotenoid products by culturing the genetically modified plants. The present invention further relates to the expression of optimized ketolase coding sequences alone or in combination with beta-cyclase coding sequences under control of an Antirrhinum majus ANTHIRRHINUM FIDDLEHEAD (AFI) promoter, In addition the invention relates to the use of an AFI promoter for heterologous gene expression, preferably for flower-specific expression of genes in plants of the genus Tagetes, to the genetically modified plants of the genus Tagetes, and to a process for producing biosynthetic products by cultivating the genetically modified plants.

The present invention relates to optimized ketolase coding sequences, corresponding coding sequences and genetic constructs alone or in combination with beta-cyclase coding sequences, their use for the expression in plants, in particular in plants of the genus Tagetes, to such genetically modified plants, to a process for the preparation of carotenoid products by culturing the genetically modified plants. The present invention further relates to the expression of optimized ketolase coding sequences alone or in combination with beta-cyclase coding sequences under control of an Antirrhinum majus ANTHIRRHINUM FIDDLEHEAD (AFI) promoter, In addition the invention relates to the use of an AFI promoter for heterologous gene expression, preferably for flower-specific expression of genes in plants of the genus Tagetes, to the genetically modified plants of the genus Tagetes, and to a process for producing biosynthetic products by cultivating the genetically modified plants.

### TECHNICAL BACKGROUND

Carotenoids are synthesized de novo in bacteria, algae, fungi and plants. In recent years, it has increasingly been attempted also to utilize plants as production organisms for fine chemicals, in particular for vitamins and carotenoids.

A natural mixture of the carotenoids lutein and zeaxanthin is extracted, for example, from the flowers of marigold plants (Tagetes plants) as “oleoresin”. This oleoresin is used both as an ingredient of food supplements and in the feed sector.

Lycopene from tomatoes is likewise used as a food supplement, while phytoene is mainly used in the cosmetic sector.

Ketocarotenoids, that is carotenoids which comprise at least one keto group, such as, for example, astaxanthin, canthaxanthin, echinenone, 3-hydroxyechinenone, 3′-hydroxyechinenone, adonirubin and adonixanthin are natural antioxidants and pigments which are produced by some algae, plants and microorganisms as secondary metabolites.

On account of their color-imparting properties, the ketocarotenoids and in particular astaxanthin are used as pigmenting aids in animal nutrition, in particular in trout, salmon and shrimp farming.

An economical biotechnological process for the production of natural, biosynthetic products and in particular carotenoids is therefore of great importance.

WO 98/18910 describes the synthesis of ketocarotenoids in nectar glands of tobacco flowers by introducing a ketolase gene into tobacco

WO 00/32788 describes some carotenoid biosynthesis genes from plants of the genus Tagetes and discloses how genetically modified plants of the genus Tagetes could be produced in order to obtain various carotenoid profiles in the petals and thus to produce certain carotenoids selectively. To this end, it was necessary to over-express some biosynthesis genes and to suppress others.

For the over expression of the newly found carotenoid biosynthesis genes in plants of the genus Tagetes, WO 00/32788 postulates the petal-specific promoter of the ketolase from Adonis vernalis.

WO05/019460 describes the use of promoters selected from EPSPS promoter, B-gene promoter, PDS promoter and CHRC promoter for expressing genes in Tagetes.

WO07/144,342 describes the use of a plastid-lipid associated protein promoter (PAP-promoter) for expressing genes in Tagetes petals.

Efremova et al. (2004. Plant Mol. Biol. 56:821-837) describe the promoter of Antirrhinum majus ANTHIRRHINUM FIDDLEHEAD (AFI) gene and the specificity directed by said promoter in trans-genic Antirrhinum and Arabidopsis but not Tagetes plants. Expression is detected in the epidermis; including petal epidermis.

Methods for the production of ketocarotenoids in plants with ketolase activity in petals are disclosed in WO 2004/018693. This previous application describes the expression of a beta carotene ketolase from different species in lutein-containing Tagetes plants. Lutein is the major carotenoid but is not being considered as the primary and desired substrate for beta-carotene ketolases to synthesize astaxanthin. Via silencing of the epsilon-cyclase gene expression, an early step in the lutein biosynthetic pathway, lutein concentrations were reduced and beta-carotenoids as substrates for ketolases increased, but only slightly. Those plants showing reduced lutein concentrations were crossed with plants accumulating low ketocarotenoid amounts. Thereby the accumulation of ketocarotenoids could be further increased.

WO2008/058946 describes methods for the production of ketocarotenoids in plants by expression of wild-type ketolase genes in petals of Tagetes. This application gives a list of additional genes the ketolase are preferentially coexppressed with to further enhance the ketocarotenoid content of the petals.

There is a constant need to make available further improved methods for the production of carotenoids, in particular ketocarotenoids, in plants, as for example plants of the genus Tagetes.

### SUMMARY OF THE INVENTION

The above-mentioned problem could be solved by the present invention, which makes use of optimized carotene ketolase coding sequences and optimized carotene ketolase coding sequences in combination with beta-cyclase sequences, which result in a surprisingly favorable production of carotenoids, in particular ketocarotenoids, in plants, in particular flowers of plants, like those of the genus Tagetes in particular, when at least one of the optimized carotene ketolase coding sequences and/or beta-cyclase sequences is expressed under the control of and AFI promoter.

The use of an AFI promoter for heterologous expression of genes in plants of the genus Tagetes has been found.

The use is particularly suitable for the flower-specific and particularly preferably for the petal-epidermis-specific heterologous expression of genes in plants of the genus Tagetes. The AFI promoter from Antirrhinum is particularly suitable for accumulating novel ketocarotenoids, not previously present in Tagetes, in i) relatively high concentration and ii) preferably in the epidermis.

### DETAILED DESCRIPTION OF THE INVENTION

### a) General Definitions

“Carotenoids”: any carotenoid, in particular alpha- and beta-carotenoids and ketocarotenoids, in particular ketocarotenoids and mixtures thereof with alpha- and/or beta-carotenoids. For example, alpha carotenoids are selected from alpha-carotene, alpha-cryptoxanthin and/or lutein; beta-carotenoids are selected from beta-carotene, beta-cryptoxanthin, zeaxanthin, antheraxanthin, violaxanthin and/or neoxanthin; ketocarotenoids are selected from astaxanthin, adonixanthin, adonirubin, echinenone, 3′-hydroxyechinenone, 3-hydroxyechinenone and/or canthaxanthin without being restricted thereto.
A preferred group of carotenoids are ketocarotenoids, especially astaxanthin.
“Carotenoids” also comprises precursors thereof as for example geranylgeranylpyrophosphate, phytoenediphosphate and phytoene.
“Carotenoids” also comprise derivatives thereof, for example derivatives of ketocarotenoids, such as esters, for example ketocarotenoid esters. The “ester” of a carotenoid means any ester, for example mono-, di- and polyester, in particular diester or a mixture of various esters. Di- or polyesters may be derived from identical or different carboxylic acids.
Esters are, in particular, esters of fatty acids. Fatty acid esters are for example composed of straight-chain or branched, mono- or polyunsaturated, optionally substituted C₆-C₃₀ monocarboxylic acids. Examples of saturated unbranched fatty acids are caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecaonic acid, arachic acid, behenic acid, lignoceric acid, cerotitic acid and melissic acid. Examples of monounsaturated fatty acids are palmitoleic acid, oleic acid and erucic acid. Examples of disaturated fatty acids are sorbic acid and linoleic acid. Examples of triunsaturated fatty acids are linolenic acid and elaeostearic acid. Examples of tetra- and polyunsaturated fatty acids are arachidonic acid, clupanodonic acid and docosahexaeonic acid. Unbranched, saturated fatty acids are preferred. Also preferred are monobasic, saturated or mono-, di- or triunsaturated C₁₀₋₂₄, preferably C₁₂₋₂₀ or C₁₄₋₂₀ fatty acids.
Carotenoids such as ketocarotenoids and their derivatives, such as for example esters as defined above include these compounds both in isomerically pure form and in the form of mixtures of stereo-isomers.
“Total carotenoids”: the amount of all carotenoids and carotenoid esters as defined above.
“Ketolase enzyme”: protein with the enzymatic activity of introducing a keto group at the optionally substituted beta-ionone ring of carotenoids, in particular, a protein with the enzymatic activity of converting beta-carotene into canthaxanthin.
“Ketolase activity”: enzymatic activity of a ketolase enzyme. Accordingly, “ketolase activity” is understood as meaning the amount of beta-carotene converted, or the amount of canthaxanthin formed, by the protein ketolase within a certain period of time.
“Beta-cyclase enzyme”: protein with the enzymatic activity of forming a beta-ionone ring on one or both ends of a linear lycopene molecule, in particular, a protein with the enzymatic activity of forming a beta-ionone ring on one or both ends of a linear lycopene molecule.
“Beta-cyclase activity”: enzymatic activity of a beta-cyclase enzyme. Accordingly, “beta-cyclase activity” is understood as meaning forming a beta-ionone ring on one or both ends of a linear lycopene molecule, by the protein beta-cyclase within a certain period of time.
“B-Gene enzyme”: protein with the enzymatic activity of forming a beta-ionone ring on one or both ends of a linear lycopene molecule, in particular, a protein with the enzymatic activity of forming a beta-ionone ring on one or both ends of a linear lycopene molecule.
“B-Gene activity”: enzymatic activity of a B-gene enzyme. Accordingly, “B-gene activity” is understood as meaning forming a beta-ionone ring on one or both ends of a linear lycopene molecule, by the protein B-gene within a certain period of time.
“Expression improved” or “expression optimized” coding sequence: sequence modified compared to the parent sequence in order to increase the expression of the encoded protein or enzyme in specific plants or part of plants such as plant plastids in comparison with the expression observed for the corresponding parent coding sequence encoding the same (i.e. substantially identical on the amino acid sequence level) protein or enzyme in the same plants or part of plants under substantially identical conditions. In particular, the parent coding sequence originates from a different, in particular non-plant, organism, and is therefore also called “heterologous coding sequence”. An “expression improved” coding sequence is codon optimized with respect to the host organism or compartment such as a tissue or plastid. Additionally for example cryptic splice sites and/or cryptic polyadenylation signals may have been avoided in the codon optimization process or removed if present in the parent sequence. Secondary structures in the transcript interfering with the translational efficiency may have also been avoided and/or removed from the sequence.
“Codon optimized”: the codon usage of the host plants, part of the host plants such as tissues or plastids is determined. Subsequently at least one codon of the non-improved sequence is changed into a codon encoding the same amino acid and having a higher abundance than the replaced codon in the host plants or plant compartments such as tissues or plastids. Preferentially, the codon with the highest abundance in the host plants or plant compartments such as tissues or plastids for the respective amino acid is chosen. More preferentially all codons of a non-improved sequence are optimized to the codon usage of the respective plants or plant compartments such as tissues or plastids.

“Non-improved” or “wild type” coding sequence, amino acid sequence or sequence: the sequence of a gene, protein or both as identified in any organism being unaltered in terms of the nucleic acid and/or amino acid sequence.

“Parent sequence”: a sequence being used as a template for any sequence alteration such as for example deletions, insertions and/or substitutions, in particular for expression improvement such as for example codon optimization. A “parent sequence” usually is a “non-improved” or “wild type” sequence but may also mean a changed for example improved sequence which is used as a template for further improvement of the coding region.
The term “substitution” means the exchange of one or more nucleotides for one or more other nucleotides. “Deletion” is the replacement of a nucleotide by a direct linkage. Insertions are introductions of nucleotides into the nucleic acid sequence, where there is formal replacement of a direct linkage by one or more nucleotides.
“Expression improved heterologous carotene ketolase coding sequence” or, as used herein synonymously Ki-sequence: a carotene ketolase coding sequence identified in a non host organism which codon usage has been optimized for the respective host plants or plant compartments such as tissues or plastids codon usage and/or where cryptic splice sites, polyadenylation signals and/or secondary structures of the transcript have been removed or avoided.
“K-sequence”: a carotene ketolase coding sequence.
“Ki-sequence”: a heterologous carotene ketolase coding sequence identified in a non host organism which codon usage has been optimized for the respective host plants or plant compartments such as tissues or plastids codon usage and/or where cryptic splice sites, polyadenylation signals and/or secondary structures of the transcript have been removed or avoided.
“K-enzyme”: protein with the enzymatic activity of introducing a keto group at the optionally substituted beta-ionone ring of carotenoids, in particular, a protein with the enzymatic activity of converting beta-carotene into canthaxanthin.
“Ki-enzyme”: protein encoded by an expression improved heterologous carotene ketolase coding sequence with the enzymatic activity of introducing a keto group at the optionally substituted beta-ionone ring of carotenoids, in particular, a protein with the enzymatic activity of converting beta-carotene into canthaxanthin.
“Bc-sequence”: a heterologous beta-cyclase coding sequence.
“Bci-sequence”: a heterologous beta-cyclase coding sequence identified in a non host organism which codon usage has been optimized for the respective host plants or plant compartments such as tissues or plastids codon usage and/or where cryptic splice sites, polyadenylation signals and/or secondary structures of the transcript have been removed or avoided.
“Bc-enzyme”: beta-cyclase protein with the enzymatic activity of forming a beta-ionone ring on one or both ends of a linear lycopene molecule.
“Bci-enzyme”: beta-cyclase protein encoded by an expression improved heterologous beta-cyclase coding sequence with the enzymatic activity of forming a beta-ionone ring on one or both ends of a linear lycopene molecule.
“Signals and/or structures negatively interfering with expression efficiency”: any sequence interfering with the expression efficiency, for example cryptic polyadenylation signals, cryptic splice sites and/or secondary structures in the transcript that are hindering the efficient translation of the protein from the transcript or negatively influence transcript stability.
“Plant tissue”: any tissue of plants, for example specialized tissues such as mesophyll, phloem or apical meristem. Additionally it covers plant organs such as for example leaf, root, flower or seed.
“Plastid”: any type of plastids present in plants such as for example proplastids, chloroplasts and/or chromoplasts.
“Plant” or “plants” as used herein are interchangeable. A method applied to a plant or an effect detectable in a plant is also applicable or detectable in a plurality of plants. The use of the word “plants” is not excluding that the respective information holds also true for a single plant.
“Part of a plant”: any part of a plant such as a single cell or a plant organ such as leaf or root. For example those parts of a plant used in transformation processes such as explants, cotyledons and cuttings. The phrase also covers harvested parts of a plant such as for example flowers, fruits, tubers and/or seeds.
“Wild type plant”: any plant that is not genetically modified, for example that is not transgenic or mutagenized.
“Starting plant”: a plant used in total or as donor plant for explants or seeds for example for trans-formation processes or mutagenization. The “starting plant” could be a “wild type plant” likewise it may also be a genetically modified plant used for supertransformation.
“Reference plant”: any plant that is used as a reference for genetically modified plants, for example transgenic or mutagenized plants. A reference plant preferentially is substantially identical to, more preferential a clone of the starting plant used in the respective process for transformation or mutagenization as defined above. A reference plant may also be a transgenic plant comprising an expression construct which itself comprises a parent sequence. This plant may be used as reference for a transgenic plant comprising the respective expression improved sequence comprised in a corresponding expression construct.
“Expression construct”: a coding region under control of and physically linked to at least those regulatory elements necessary and sufficient for expression of the coding region in plants.
“Regulatory element”: any element able to regulate transcription and/or translation of a sequence and comprises for example promoters, enhancers, polyadenylation signals, terminators and/or regulatory introns. A person skilled in the art is aware of other regulatory regions described in the state of the art useful for regulating expression of a coding region in an appropriate way.
“Promoter”: a nucleic acid having expression activity, and thus means a nucleic acid which, functionally linked upstream of a nucleic acid to be expressed, also referred to as gene hereinafter, regulates the expression, that is the transcription and the translation, of this nucleic acid or of this gene.
“Plant specific promoter”: any promoter able to generate expression in plants irrespective of the origin of the promoter. It could be derived for example from a plant, an alga, a bacteria, a plant virus, a plastid or could be a synthetic sequence.
“Tissue specific promoter”: a plant specific promoter as defined above generating expression for example specifically, predominantly or preferably in specific plant tissues.
“Plastid specific promoter”: any promoter capable of generating expression in plant plastids irrespective of the origin of the promoter. It could be derived for example from a plant, an alga, a bacteria, a plant virus, a plastid or could be a synthetic sequence.
“Constitutive” promoter: promoters which ensure expression in a large number or, preferably all tissues over a substantial period of the plant's development, preferably at all points in time of the plant's development.

The ketolase activity in genetically modified plants of the invention and in wild type or reference plants is determined under the following conditions: The ketolase activity in plant materials is determined as described in Frazer et al., (J. Biol. Chem. 272(10): 6128-6135, 1997). The ketolase activity in plant extracts is determined using the substrates beta-carotene and canthaxanthin in the presence of lipid (soya lecithin) and detergent (sodium cholate). Substrate/product ratios from the ketolase assays are determined by HPLC.

In the case of an “increased” ketolase activity, the amount of beta-carotene converted, or the amount of canthaxanthin formed by the protein ketolase within a certain period of time is higher than that converted or formed by a protein ketolase in the reference plant. Preferably the “increase” of the ketolase activity amounts to at least 5%, furthermore preferably at least 20%, furthermore preferably at least 50%, furthermore preferably at least 100%, more preferably at least 300%, even more preferably at least 500%, in particular at least 600% of the ketolase activity of the respective reference plant.

The beta-cyclase activity in genetically modified plants of the invention and in wild type or reference plants is determined as described in Frazer et al., (J. Biol. Chem. 272(10): 6128-6135, 1997), using Potassium phosphate buffer (pH7.6), with lycopin as substrate, and adding stroma proteins from paprika, NADP+, NADPH and ATP. The beta-cyclase activity in plant extracts is determined as described in Frazer et al., (J. Biol. Chem. 272(10): 6128-6135, 1997) Substrate/product ratios from the beta-cyclase assays are determined by HPLC.

In the case of an “increased” beta-cyclase activity, the amount of lycopene converted, or the amount of beta-carotene formed by the protein beta-cyclase within a certain period of time is higher than that converted or formed by a beta-cyclase protein in the reference plant. Preferably the “increase” of the beta-cyclase activity amounts to at least 5%, furthermore preferably at least 20%, furthermore preferably at least 50%, furthermore preferably at least 100%, more preferably at least 300%, even more preferably at least 500%, in particular at least 600% of the beta-cyclase activity of the respective reference plant.

“Essentially quantitative hydrolytic ester cleavage”: at least one of the carotenoid esters present, in particular at least one of the ketocarotenoid esters present, is at least about 85%, in particular at least about 88%, hydrolyzed by enzymatic activity according to the invention so that ester groups are no longer present in the molecule.
“Hydrolysis rate”: the percentage decrease in the amount of (e.g. extracted) carotenoid esters in a reactant. The hydrolysis rate can be determined in particular by determining the carotenoid ester content in the reactant before and after the hydrolytic treatment, e.g. by chromatography as described in the examples, and determining the content of hydrolyzed esters therefrom. The hydrolysis rates may be 100% or less, that is no carotenoid esters remain or a certain amount remain. For example the hydrolysis rate may range from 88% to 99% or from 95% to 99%.
“Carotenoid content” is defined as the amount of total carotenoids determined by HPLC.
“Carotenoid ester content”: the amount of carotenoids esterified by a saturated or mono- or di- or triunsaturated C₁₀₋₂₄, preferably C₁₂₋₂₀ or C₁₄₋₂₀ monocarboxylic acid. The content of carotenoid esters can be measured inter alia by chromatography because carotenoid esters usually have longer retention times on suitable reverse phase support materials such as, for example, long-chain polymer-bound C30 phases than do unbound carotenoids. A suitable C₃₀ support material and suitable separation conditions are mentioned by way of example in example 3.
“Carotenoid profile”: the relative amount of different carotenoids compared to each other determined in plants or part of plants.
“Microorganisms”: bacteria, yeasts, algae or fungi.
“Expression activity”: the amount of transcript formed in a certain time from a gene or the amount of protein formed in a certain time from a transcript or both.
“Increased expression activity” or “increased expression rate”: the formation of an increased transcript and/or protein amount formed from a gene in modified plants during a certain period of time, in comparison with reference plants, e.g. wild type.
“Coexpression”: coordinated expression of at least two transgenes in one plant, for example the expression of at least two transgenes in the same tissue at the same developmental stage of said tissue or for example the expression of at least two transgenes coordinated in a timely sequence. The at least two transgenes might be expressed from one construct or by independent constructs.

### b) Particular Aspects of the Present Invention

One aspect of the present invention relates to a process for the preparation of at least one carotenoid, in particular at least one ketocarotenoid as defined above, in genetically modified plants, which method comprises expressing in plants at least one carotene ketolase enzyme encoded by an expression improved heterologous carotene ketolase coding sequence (Ki-sequence) or at least one Ki-sequence and a Bc-sequence the latter may be a wild-type sequence or an expression improved sequence. In particular, an “increased expression activity” or “increased expression rate” of the coding sequence or gene is observed, resulting in the increased formation of at least one carotenoid as defined above in the plants. In particular, the content of at least one ketocarotenoid selected from astaxanthin, adonixanthin, adonirubin, echinenone, 3′-hydroxyechinenone, 3-hydroxyechinenone, canthaxanthin, and/or the total content of ketocarotenoids in genetically modified plants or parts thereof is statistically significantly increased, if compared to reference plants. Statistically significantly increased means that at least three independent transgenic lines and three reference plants, e.g. wild type plants, are compared for their total carotenoid content and that the amount of total carotenoids in transgenic lines are significantly higher using a statistical test such as the t-test. In the context of ketocarotenoids that are not detectable in most plants for example Tagetes plants the respective detection limit is defined by measuring a series of dilutions of the respective compound with the respective method applied to the plant according to the invention. The detection limit is taken as the value for the total ketocarotenoid content of the reference plants, e.g. wild type plants and the statistical test is accordingly applied to test the significance of the total ketocarotenoid content increase.

According to the process of the invention the ketolase activity or the ketolase activity and the beta-cyclase activity may also be increased by applying at least one of the following methods:

The ketolase activity or the ketolase activity and the beta-cyclase activity can be further increased in various ways, for example by eliminating inhibiting regulatory mechanisms at the translation and protein level, or by increasing the gene expression of a nucleic acid encoding a ketolase or a ketolase and a beta-cyclase in comparison with the reference plant, e.g. wild type, for example by inducing the ketolase gene or both the ketolase gene and the beta-cyclase gene by activators.

An increase of the gene expression of a nucleic acid encoding a ketolase or a ketolase and a beta-cyclase is also understood as meaning the manipulation of the expression of the plants' homologous endogenous ketolases or endogenous ketolases and endogenous beta-cyclases. This can be achieved for example by modifying the promoter DNA sequence of the respective genes. Such a modification, which results in a modified, preferentially increased expression rate of at least one endogenous ketolase gene or at least one endogenous ketolase gene and at least one endogenous beta-cyclase gene, can be a deletion, insertion or substitution of DNA sequences.

It is also possible to modify the expression of at least one endogenous ketolase or at least ketolase and at least one beta-cyclase by applying exogenous stimuli. This can be carried out by specific physiological conditions, i.e. by the application of foreign substances.

Moreover, an increased expression of at least one endogenous ketolase gene or at least one ketolase gene and at least one beta-cyclase gene can be achieved by a regulator protein, which does not occur in the reference plant, e.g. wild type, or which is modified, and which interacts with the promoter of these genes.

Such a regulator can constitute a chimeric protein, which consists of a DNA binding domain and a transcription activator domain such as described, for example, in WO 96/06166.

In addition, there may be at least one further ketolase gene or at least one further ketolase gene and at least one further beta-cyclase present in the genetically modified plants according to the invention in comparison with the reference plants, e.g. wild type. In this aspect, the genetically modified plants according to the invention, accordingly, have at least one exogenous (=heterologous) nucleic acid encoding a ketolase or at least one exogenous nucleic acid encoding a ketolase gene and at least one exogenous nucleic acid encoding a beta-cyclase, or at least two endogenous nucleic acids encoding ketolases or at least two endogenous nucleic acids encoding ketolases and at least two endogenous nucleic acids encoding beta-cyclases.

There may also be that the starting plants used are plants, which show no ketolase activity or no ketolase and no beta-cyclase activity in the plants, in particular in petals.

The beta-cyclase enzyme coexpressed with the improved ketolase enzyme can be encoded by a wild-type sequence or a expression improved sequence.

Preferably, the content of at least one of the ketocarotenoids, or the total ketocarotenoid content as defined above, is increased by at least 1%, as for example from 2 to 100%, or by a factor of 1 to 10, as for example 1, 2, 3, 4 or 5.

Preferably, the plants are of one of the following families: Ranunculaceae, Berberidaceae, Begoniaceae, Papaveraceae, Cannabaceae, Chenopodiaceae, Cruciferae, Rosaceae, Fabaceae, Linaceae, Vitaceae, Brassiceae, Cucurbitaceae, Primulaceae, Caryophyllaceae, Amaranthaceae, Apocynaceae, Balsaminaceae, Gentianaceae, Geraniaceae, Graminae, Euphorbiaceae, Labiatae, Leguminosae, Caprifoliaceae, Oleaceae, Tropaeolaceae, Solanaceae, Lobeliaceae, Scrophulariaceae, Compositae, Asteraceae, Plumbaginaceae, Liliaceae, Amaryllidaceae, Rubiaceae, Poaceae, Polemoniaceae, Orchidaceae, Umbelliferae, Verbenaceae, Violaceae, Malvaceae, Illiaceae and Lamiaceae.

In particular plants are selected from the genus Tagetes, Acacia, Aconitum, Adonis, Arnica, Aqulegia, Aster, Astragalus, Bignonia, Calendula, Caltha, Campanula, Canna, Centaurea, Chemanthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hisbiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola and Zinnia.

Preferably the plants are selected from the genus Tagetes (as for example Tagetes erecta and Tagetes patula).

Likewise it is also possible to perform the claimed process with plants showing a low lutein (an alpha-carotenoid) content. This may be achieved by directed or non-directed mutagenesis in combination with screening for mutants with the desired carotenoid profile. Non-directed mutagenesis, for example, may be achieved by making use of chemical mutagens, as for example EMS (ethyl methane sulfonate). Corresponding methods for obtaining such plants, in particular lutein-depleted plants (lutein content 0 to about 90% based on the total carotenoid content, see U.S. Pat. No. 6,784,351), are known in the art, and one specific method is referred to in the experimental part.

Preferably the process of the invention is performed such that the expression of the ketolase gene or the ketolase gene and the beta-cyclase gene is, primarily or specifically, observed in flowers of the plants, in particular in petals, especially in plastids like chromoplasts, of the plants. The latter can be achieved for example by targeting the ketolase protein or the ketolase protein and the beta-cyclase protein to the respective organelle or by expressing the ketolase or the ketolase and the beta-cyclase in the respective organelle for example by plastid transformation.

The Ki-sequence or both the Ki-sequence and the Bci-sequence may preferably be modified by adapting its codon usage to the codon usage of the plants or compartments of the plants such as tissues or plastids such as for example chromoplasts. In addition other methods for improving translational efficiency (i.e. number of proteins translated from one transcript per time) may be applied to the parent sequence such as reducing the number, the length and/or the binding energy of potential secondary structures in the transcript, avoiding or removing cryptic splice sites and/or the number of cryptic polyadenylation signals.

In order to adapt the codon usage of a coding sequence to the codon usage of a host organism, the respective host's codon usage may be determined. A list of codon usages for a large range of organisms and organelles may for example be found in the resources of the Japanese “Kasuza DNA Research Institute” provided in the internet under http://www.kazusa.or.jp/codon/. The person skilled in the art is aware of methods of how to define the codon usage of any given organism or organelle which in addition is exemplified for the determination of the codon usage of Tagetes in the example 4 below. This procedure may be applied to any other organism.

In particular, the adaptation of the coding sequences to the codon usage of the plants or compartments of the plants such as tissues or plastids comprise replacing at least one parent sequence codon by a different replacement codon encoding the same amino acid, resulting in an “increased expression activity” or “increased expression rate” of the respective gene.

Preferably the replacement codon (to be introduced) is present in the genome or transcriptome of the plants or compartments of the plants such as tissues or plastids such as for example chromoplasts in an abundance, which is identical to or higher than the abundance of the parent sequence codon (to be replaced).

In particular, at least one codon of the parent ketolase coding sequence or both the parent ketolase coding sequence and the parent beta-cyclase coding sequence is adapted to a higher abundant codon, preferentially the most abundant codon for the respective amino acid.

The codon usage may be adapted such that the least abundant codons of any amino acid are adapted to higher abundant codons for the respective amino acids, preferentially the codon of the parent sequence is adapted to the most abundant codon for the respective amino acid.

The codon usage of the parent sequence may also be adapted in a way that the codon usage of the respective expression improved sequence resembles the host's codon usage.

The codon usage may be adapted such that 10 to 100%, as for example 50 to 100%, or 90 to 100%, of the codons of the ketolase gene are adapted to the codon usage of the plants or plant plastids such as for example chromoplasts. In particular, the most abundant codon for the respective amino acid is used for the adaptation.

In addition, the expression of the ketolase gene or both the ketolase gene and the beta-cyclase gene may be further improved by removing or avoiding signals and/or structures in the sequence negatively interfering with expression efficiency in the respective host organism such as higher plants or plant plastids such as for example chromoplasts, for example by removing cryptic splice sites, cryptic polyadenylation signals or sequences able to form secondary structures inhibiting expression in particular translation. The person skilled in the art is aware of methods on how to identify and avoid or remove the respective signals such as cryptic splice sites (Haseloff et al. (1997) PNAS 94, 2122-2127), cryptic polyadenylation signals (Grec et al. (2000) Gene 242, 87-95; Rutherford et al. (2005) Plant Journal 43, 769-788) or secondary structures interfering with expression (Wang and Wessler (2001) Plant Phys. 125, 1380-1387) with no or only minor changes of the protein sequence encoded by the respective coding sequence, for example only leading to exchange of similar amino acids as for example shown in the table given in chapter g). Preferentially the respective signals and structures are removed or avoided without changing the amino acid sequence of the protein encoded by the respective coding sequence.

The expression-improved heterologous carotene ketolase coding sequence may be derived from a corresponding parent sequence, e.g. wild type coding sequence of prokaryotic or eukaryotic origin, as for example derived from a corresponding wild type sequence of bacteria, yeast or algae. In particular the coding sequence is derived from the corresponding wild type sequences of algae, in particular algae of the genus Haematococcus, Chlamydomonas, Scenedesmus, or Chlorella.

Non-limiting examples of preferred algal species are Haematococcus pluvialis, Chlamydomonas reinhardtii, Scenedesmus vacuolatus or Chlorella zoofingiensis.

Preferably, a ketolase enzyme is expressed having an amino acid sequence encoded by the corresponding coding portions comprised a nucleotide sequence selected from SEQ ID NO: 3, 4, 7, 8, 10, 11, 13 and 14; or coding sequences derived therefrom by nucleic acid substitution, addition, deletion or insertion, and encoding a ketolase enzyme having a sequence identity of at least 50%, as for example at least 60%, at least 70%, at least 80% or 90% to 100% or 95% to 99%, with respect to the parent sequence and retaining ketolase activity.

Preferably, the expression-improved ketolase coding sequence is selected from the corresponding coding portions comprised in a nucleotide sequence selected from SEQ ID NO: 3, 4, 7, 8, 10, 11, 13 and 14; or coding sequences derived therefrom by nucleic acid substitution, addition, deletion or insertion, and encoding a ketolase enzyme having a sequence identity of at least 50%, as for example at least 60%, at least 70%, at least 80% or 90% to 100% or 95% to 99%, with respect to the parent sequence and retaining ketolase activity.

The beta-cyclase coding sequence or the expression-improved heterologous beta-cyclase coding sequence may be derived from a corresponding parent sequence, e.g. wild type coding sequence of prokaryotic or eukaryotic origin, as for example derived from a corresponding wild type sequence of a algae or plant. In particular the coding sequence is derived from the corresponding wild type sequences of a plant, in particular plants of the genus Solanacea.

A non-limiting example of a preferred plant species is Lycopersicon esculentum.

Preferably, a beta-cyclase enzyme is expressed having an amino acid sequence encoded by the corresponding coding portions comprised on a nucleotide sequence selected from SEQ ID NO: 15, 17 or 25; or coding sequences derived therefrom by nucleic acid substitution, addition, deletion or insertion, and encoding a ketolase enzyme having a sequence identity of at least 50%, as for example at least 60%, at least 70%, at least 80% or 90% to 100% or 95% to 99%, with respect to the parent sequence and retaining beta-cyclase activity.

Preferably, the expression-improved beta-cyclase coding sequence is selected from the corresponding coding portions comprised in a nucleotide sequence selected from SEQ ID NO: 17; or coding sequences derived therefrom by nucleic acid substitution, addition, deletion or insertion, and encoding a ketolase enzyme having a sequence identity of at least 50%, as for example at least 60%, at least 70%, at least 80% or 90% to 100% or 95% to 99%, with respect to the parent sequence and retaining beta-cyclase activity.

The genetically modified plants of the process may carry expression constructs encompassing at least one Ki-sequence or at least one Ki-sequence and at least one Bc-sequence or at least two expression constructs one carrying at least one Ki-sequence and the other carrying at least one Bc-sequence under the control of suitable regulatory elements.

Preferably, the Ki-sequence or both the Ki-sequence and Bc-sequence is under the control of a plant specific, in particular plant plastid or plant tissue specific promoter.

In particular, the promoter directs the flower specific, in particular petal specific expression.

The expression of the expression improved ketolase or both the expression improved ketolase and beta-cyclase may alternatively take place in the plastids. In that case a promoter functional in plant plastids especially chromoplasts is preferred for controlling expression of the respective enzyme.

The expression construct may further comprise the coding sequence for a transit peptide, operably linked to the coding sequence of the enzymes. The transit peptide may originate, from a different organism, and the coding sequence of such a heterologous transit peptide preferably may be adapted to the codon usage of the plants or compartments of the plants such as tissues or plastids such as for example chromoplasts as explained above.

The present invention also relates to expression constructs as defined above, recombinant vectors and/or microorganisms comprising at least one of the expression constructs.

The present invention also relates to genetically modified plants carrying at least one Ki-sequence as defined above, or at least one expression construct as defined above, or at least one vector as defined above.

The present invention further relates to genetically modified plants expressing carotene ketolase activity in at least one plant tissue or plastid, and, in particular, genetically modified plants expressing carotene ketolase activity in their flowers, flower petals and/or chromoplasts.

A further aspect of the invention relates to genetically modified plants having an altered carotenoid profile, in particular in their flowers, preferably in their petals, in particular in their plastids.

A further aspect of the invention relates to genetically modified plants containing a detectable amount of at least one ketocarotenoid in at least one part of the plants, in particular in their flowers, preferably petals. The genetically modified plants are selected from plants of the families and the genus as defined above.

A further aspect of the invention also relates to parts or seeds of genetically modified plants as defined above.

A further aspect of the invention relates to a process of preparing genetically modified plants as defined above, which process comprises introducing into the plants at least one expression construct as defined above into starting plants.

A further aspect of the invention relates to a process of preparing parts or seeds of genetically modified plants as defined above, which process comprises introducing into the plants at least one expression construct as defined above into starting plants, growing the so obtained plants and obtaining parts or seeds thereof.

A further aspect of the invention relates to a process of preparing at least one carotenoid, in particular ketocarotenoid, which process comprises cultivating genetically modified plants as defined above under conditions which allow the expression of improved ketolase activity or of improved ketolase activity and beta-cyclase activity for a sufficient time to produce a detectable amount of at least one ketocarotenoid or derivative thereof within the plants, and isolating the ketocarotenoid or derivative thereof.

A further aspect of the invention relates to the use of genetically modified plants as defined above for preparing carotenoids, in particular ketocarotenoids.

Another aspect of the invention relates to an expression-improved carotene ketolase coding sequence as defined above.

Another aspect of the invention relates to an expression-improved beta-cyclase coding sequence as defined above.

In another aspect, the present invention relates to a process of chemically hydrolyzing carotenoid esters obtained by extracting carotenoid ester containing plants for example such as described above, which process comprises hydrolyzing the carotenoid esters under substantially anaerobic conditions. Preferably the substantially anaerobic hydrolysis is performed in the presence of a base and/or at a reduced reaction temperature. Substantially anaerobic means the absence of oxygen from the reaction medium or that essentially no oxygen is present in the reaction medium. In general an oxygen content of the reaction medium in the range of 1 to 50 ppm, as for example 1 to 10 ppm is acceptable.

In particular, the base is used in a concentration in the range of 0.01 to 0.5 M, as for example 0.1 to 0.4 M. Non-limiting examples of suitable bases are alkali metal salts, as for example sodium or potassium alkoxides, as for example sodium methoxide.

The reaction temperature is in the range from minus 10 to plus 20° C., as for example from 0 to plus 10° C. or from plus 5 to plus 9° C.

The carotenoid esters to be hydrolyzed may be obtained by extracting carotenoid ester containing plants or parts thereof, as for example flowers of parts of the flowers, with organic solvents for example acetone, and optionally removing the solvent.

In another aspect the present invention provides a process of analyzing carotenoids in plants, which process comprises obtaining a sample of plant material, as for example of specific parts of the plants, like flowers or parts thereof, like petals, isolating a carotenoid ester containing sample therefrom, performing a chemical hydrolysis of the carotenoid esters as defined above and determining the carotenoid content of the hydrolyzed product in a manner known per se, as for example chromatographically.

### c) Ketolase Coding Sequences

Suitable ketolase coding sequences which may be used as parent sequence and therefore expression improved according to the invention and applied in a process of the invention are summarized in Annex 1 and described via their data base entries. Preferred ketolase coding sequences are sequences derived from the species Haematococcus pluvialis, Chlamydomonas reinhardtii, Scenedesmus vacuolatus or Chlorella zoofingiensis, especially preferred are the ketolase coding sequences as shown in SEQ ID NO: 3, 7, 10, 13, 20 and 30.

### d) Beta-Cyclase Coding Sequences

Suitable Beta-cyclase coding sequences which may be expressed in combination with the above mentioned ketolase sequences or may be used as parent sequence and therefore expression improved according to the invention and applied in a process of the invention are summarized in Annex 2 and described via their data base entries. Preferred beta-cyclase coding sequences are chromoplast specific. The especially preferred beta-cyclase is the B-gene coding sequences as shown in SEQ-ID 15.

### e) Promoter Sequences

The invention additionally relates to AFI promoters and their use in transgenic plants, preferentially transgenic Tagetes plants, especially for expression of ketolase and/or beta-cyclase genes in transgenic Tagetes plants.

The use is particularly suitable for the flower-specific and particularly preferably for the petal-specific heterologous expression of genes in plants of the genus Tagetes. The AFI promoter from Anthirrhinium is particularly suitable for accumulating novel ketocarotenoids, not previously present in Tagetes, in i) relatively high concentration and ii) preferably in the epidermis.

Moreover, the invention relates to the use of AFI promoters in transgenic Tagetes plants wherein the expression takes place in epidermis, especially in petal epidermis.

An “AFI promoter” means any promoter which naturally occurs in plants such as Antirrhinum and which cause gene expression of Antirrhinum fiddlehead protein.

Preferred AFI promoters comprise
A1) the nucleic acid sequence SEQ. ID. NO. 28 or
A2) a sequence derived from this sequence by substitution, insertion or deletion of nucleotides and having an identity of at least 60% at the nucleic acid level with the respective sequence SEQ. ID. NO. 28, or
A3) a nucleic acid sequence which hybridizes with the nucleic acid sequence SEQ ID NO. 28 under stringent conditions, or
A4) functionally equivalent fragments of the sequence under A1), A2) or A3)

The nucleic acid sequence SEQ. ID. NO. 28 represents a promoter sequence of the antirrhinum fiddlehead protein from Antirrhinum majus.

The invention further relates to AFI promoters comprising a sequence derived from the sequence (SEQ. ID. NO. 28) by substitution, insertion or deletion of nucleotides and having an identity of at least 60% at the nucleic acid level with the respective sequence SEQ ID NO. 28.

Further natural examples of the invention of AFI promoters of the invention can be easily found for example from various organisms whose genomic sequence is known by comparisons of the identity of the nucleic acid sequences from databases with the sequences SEQ ID NO. 28 described above.

Artificial AFI promoter sequences of the invention can easily be found starting from the sequences SEQ ID NO. 28 by artificial variation and mutation, for example by substitution, insertion or deletion of nucleotides.

A nucleic acid sequence having an identity of at least 60% with the sequence SEQ ID NO. 28 accordingly means a nucleic acid sequence which, on comparison of its sequence with the sequence SEQ ID NO. 28, in particular in accordance with the above programming algorithm with the below defined set of parameters, shows an identity of at least 60%.

Particularly preferred AFI promoters have an identity of at least 70%, preferably at least 80%, at least 90%, at least 92%, at least 95%, at least 96%, at least 97%, at least 98%, particularly preferably at least 99%, with the respective nucleic acid sequence SEQ ID NO. 28.

Further natural examples of AFI promoters can further easily be found starting from the nucleic acid sequences described above, in particular starting from the sequence SEQ ID NO. 28, from various organisms whose genomic sequence is unknown, by hybridization techniques in a manner known per se.

The invention therefore further relates to AFI promoters comprising a nucleic acid sequence which hybridizes with the nucleic acid sequence SEQ ID NO. 28 under stringent conditions. This nucleic acid sequence comprises at least 10, more preferably more than 12, 15, 30, 50 or particularly preferably more than 150 nucleotides.

“Hybridization” means the ability of a poly- or oligonucleotide to bind under stringent conditions to an almost complementary sequence, while nonspecific bindings between non-complementary partners do not occur under these conditions. For this, the sequences should preferably be 90-100% complementary. The property of complementary sequences being able to bind specifically to one another is made use of for example in the Northern or Southern blotting technique or in primer binding in PCR or RT-PCR.

A “functionally equivalent fragment” means for promoters fragments which have essentially the same promoter activity as the initial sequence.

“Essentially identical” means a specific expression activity which displays at least 50%, preferably 60%, more preferably 70%, more preferably 80%, more preferably 90%, particularly preferably 95%, of the specific expression activity of the initial sequence.

“Fragments” mean partial sequences of the AFI promoters described by embodiment A1), A2) or A3). These fragments preferably have more than 10, but preferably more than 12, 15, 30, 50 or particularly preferably more than 150, connected nucleotides of the nucleic acid sequence SEQ. ID. NO. 28.

It is particularly preferred to use the nucleic acid sequence SEQ. ID. NO. 28 as AFI promoter, i.e. for expressing genes in plants of the genus Tagetes.

All the aforementioned AFI promoters can further be produced in a manner known per se by chemical synthesis from the nucleotide building blocks, such as, for example, by fragment condensation of individual overlapping, complementary nucleic acid building blocks of the double helix. The chemical synthesis of oligonucleotides can take place for example in a known manner by the phosphoamidite method (Voet, Voet, 2^{nd} edition, Wiley Press New York, pp. 896-897). Addition of synthetic oligonucleotides and filling in of gaps using the Klenow fragment of DNA polymerase and ligation reactions, and general cloning methods are described in Sambrook et al. (1989), Molecular cloning: A laboratory manual, Cold Spring Harbor Laboratory Press.

It is possible with the promoters of the invention in principle for any gene to be expressed, in particular flower-specifically expressed, particularly preferably petal-specifically expressed, more preferably petal epidermis specifically expressed in plants of the genus Tagetes.

These genes to be expressed in plants of the genus Tagetes are also called “effect genes” hereinafter.

Preferred effect genes are for example genes from the biosynthetic pathway of odorous substances and flower colors whose expression or increased expression in plants of the genus Tagetes leads to an alteration of the odor and/or of the flower color of flowers of the plants of the genus Tagetes.

Particularly preferred effect genes are genes from biosynthetic pathways of biosynthetic products which can naturally be produced in plants of the genus Tagetes, i.e. in the wild type or by genetic alteration of the wild type, can be produced in particular in flowers, can be produced particularly preferably in petals.

Preferred biosynthetic products are fine chemicals. These compounds include organic acids, amino acids, lipids, saturated and unsaturated fatty acids (e.g. arachidonic acid), carotenoids, vitamins and cofactors (as described in Ullmann's Encyclopedia of Industrial Chemistry, vol. A27, “Vitamins”, pp. 443-613 (1996) VCH: Weinheim and the references present therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1-3, 1994 in Penang, Malaysia, AOCS Press (1995)).

More preferred fine chemicals or biosynthetic products which can be produced in plants of the genus Tagetes, especially in petals of the flowers of the plants of the genus Tagetes, are carotenoids such as, for example, phytoene, lycopene, beta-carotene, lutein, zeaxanthin, astaxanthin, canthaxanthin, echinenone, 3-hydroxyechinenone, 3′-hydroxyechinenone, adonirubin, violaxanthin and adonixanthin.

Very particularly preferred genes expressed with the promoters of the invention in plants of the genus Tagetes are accordingly genes which encode proteins from the biosynthetic pathway of carotenoids.

Particularly preferred genes are selected from the group of nucleic acids encoding a ketolase, nucleic acids encoding a beta-hydroxylase, nucleic acids encoding a beta-cyclase, nucleic acids encoding an epsilon-cyclase, nucleic acids encoding a zeaxanthin epoxidase, nucleic acids encoding an antheraxanthin epoxidase, nucleic acids encoding a neoxanthin synthase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate beta-isomerase, nucleic acids encoding a geranyl-diphosphate synthase, nucleic acids encoding a farnesyl-diphosphate synthase, nucleic acids encoding a geranyl-geranyl-diphosphate synthase, nucleic acids encoding a phytoene synthase, nucleic acids encoding a phytoene desaturase (phytoene dehydrogenase), nucleic acids encoding a prephytoene synthase, nucleic acids encoding a zeta-carotene desaturase, nucleic acids encoding a crtISO protein,

nucleic acids encoding a 4-diphosphocytidyl-2-C-methyl-D-erythritol synthase, nucleic acids encoding a 4-diphosphocytidyl-2-C-methyl-D-erythritol kinase,
nucleic acids encoding a 2-methyl-D-erythritol-2,4-cyclodiphosphate synthase,
nucleic acids encoding a hydroxymethylbutenyl-diphosphate synthase, nucleic acids encoding an FtsZ protein and nucleic acids encoding an MinD protein.

Examples of nucleic acids encoding a ketolase, and the corresponding ketolases, are given in Annex 1, preferred nucleic acids encoding a ketolase are the expression-improved ketolase coding sequence selected from the corresponding coding portions comprised in the nucleotide sequence selected from SEQ ID NO: 3, 4, 7, 8, 10, 11, 13, 14, 30 and 31.

Examples of beta-cyclase genes are given in Annex 2, preferred nucleic acids encoding a beta-cyclase are selected from the corresponding coding portions comprised on a nucleotide sequence selected from SEQ ID NO: 15, 17 or 25.

Hence, another aspect of the invention is the use of an AFI promoter as defined above for expression in transgenic plants wherein the AFI promoter is functionally linked to a ketolase gene and/or a beta-cyclase gene.

Further, an aspect of the invention is a genetically modified plant of the genus Tagetes, where the genetic modification leads to an increase or causing of the expression rate of at least one gene compared with the wild type and is caused by regulation of the expression of this gene in the plant by an AFI promoter as defined above.

Particularly, the invention relates to genetically modified plants, preferably plants of the genus Tagetes, wherein the regulation of the expression of genes in the plant is achieved by AFI promoters according to definitions given above by
a) introducing one or more AFI promoter of the invention into the genome of the plant, so that expression of one or more endogenous genes takes place under the control of the introduced promoters, or
b) introducing one or more genes into the genome of the plant, so that expression of one or more of the introduced genes takes place under the control of the endogenous promoters of the invention, or
c) introducing one or more nucleic acid constructs comprising at least one promoter of the invention and, functionally linked, one or more genes to be expressed into the plant.

In a preferred embodiment, according to feature c) one or more nucleic acid constructs comprising at least one promoter of the invention and, functionally linked, one or more genes to be expressed are introduced into the plant. Integration of the nucleic acid constructs in the plant of the genus Tagetes can take place in this case intrachromosomally or extrachromosomally.

Preferred promoters of the invention and preferred genes to be expressed (effect genes) are described above.

The invention further relates to a process for producing biosynthetic products by cultivating genetically modified plants of the genus Tagetes as defined above.

Another aspect of the invention relates to a process for producing carotenoids by cultivating genetically modified plants of the invention, wherein the genes to be expressed comprise at least one K-sequence, preferably one Ki-sequence and/or at least one Bc-sequence. In particular wherein the K-sequence or the Ki-sequence from Scenedesmus is used and/or as beta-cyclase a B-gene from tomato is used.

The invention further relates to a process as defined above for producing astaxanthin and astaxanthin derivatives. Especially, the invention relates to said process, wherein the genetically modified plants or parts of plants are harvested after the cultivation, and then the carotenoids are isolated from the genetically modified plants or parts of plants.

Numerous other plant specific promoters are well known in the art and are also suitable for use in the invention.

As a specific example of a suitable plant specific promoter there may be mentioned the Brassica napus plastid-associated protein X (PAPX) promoter. This promoter substantially corresponds to the nucleotide sequence of position 1734 to 2764 of SEQ ID NO:4. Further suitable promoters are functionally equivalent sequences as derived therefrom which direct the expression of a coding sequence with comparable efficiency and/or specificity. Functionally equivalent promoter sequences may be derived therefrom or may have a sequence homology or identity determinable as defined below, and being in the range of 40 to below 100%, or at least 50%, at least 60%, at least 70%, at least 80%, or at least 90%, as for example 91% to 99% or 94% to 98% if compared to the nucleotide sequence of position 1734 to 2764 of SEQ ID NO:4. Further suitable PAP promoters are disclosed in PCT/EP2007/055756, filed Jun. 12, 2007, the disclosure of which document is herein incorporated by reference. In particular, page 4 line 22 to page 8 line 10 and the sequences referred to therein are herewith incorporated by reference.

Suitable promoters are plant promoters or promoters derived from plant viruses. Specifically mentioned is the promoter of the CaMV cauliflower mosaic virus 35S transcript (Franck et al. (1980) Cell 21:285-294; Odell et al. (1985) Nature 313:810-812; Shewmaker et al. (1985) virology 140:281-288; Gardner et al. (1986) Plant Mol Biol 6:221-228) or the 19S CaMV promoter (U.S. Pat. No. 5,352,605; WO 84/02913; Benfey et al. (1989) EMBO J. 8:2195-2202).

Further suitable plant promoters are the fruit specific pds promoter (Pecker et al. (1992) Proc. Natl. Acad. Sci. USA 89: 4962-4966), the leaf preferential “Rubisco small subunit (SSU)” promoter (U.S. Pat. No. 4,962,028) or the seed specific legumin B promoter (GenBank Acc. No. X03677). Further suitable are constitutive promoters as the promoter of the Agrobacterium nopaline synthase, the TR dual promoter, the OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the mas promoter (Fox et al. (1992) Plant Molecular Biology 20 (2) 219-233), the cinnamyl alcohol dehydrogenase promoter (U.S. Pat. No. 5,683,439), the promoters of the vacuolar ATPase subunits, the promoter of a proline-rich protein from wheat (WO 91/13991), the P-nit promoter (Y07648.L, Hillebrand et al. (1998), Plant. Mol. Biol. 36, 89-99, Hillebrand et al. (1996), Gene, 170, 197-200), the ferredoxin NADPH oxidoreductase promoter (database entry AB011474, position 70127 to 69493), the TPT promoter (WO 03006660), the “superpromoter” (U.S. Pat. No. 5,955,646), the 34S promoter (U.S. Pat. No. 6,051,753), and further promoters of genes whose constitutive expression in plants is known to the skilled worker.

The expression cassettes may also comprise a chemically inducible promoter (review paper: Gatz et al. (1997) Annu Rev Plant Physiol Plant Mol Biol 48:89-108), by means of which the expression of the ketolase gene in plants can be controlled at a particular point in time. Such promoters such as, for example, the PRP1 promoter (Ward et al. (1993) Plant Mol Biol 22:361-366), salicylic-acid-inducible promoter (WO 95/19443), a benzene-sulfonamide-inducible promoter (EP 0 388 186), a tetracyclin-inducible promoter (Gatz et al. (1992) Plant J 2:397-404), an abscisic-acid-inducible promoter (EP 0 335 528) or an ethanol- or cyclohexanone-inducible promoter (WO 93/21334) can likewise be used.

Other promoters are those which are induced by biotic or abiotic stress such as, for example, the pathogen-inducible promoter of the PRP1 gene (Ward et al. (1993) Plant Mol Biol 22:361-366), the heat-inducible hsp70 or hsp80 promoter from tomato (U.S. Pat. No. 5,187,267), the cold-inducible alpha-amylase promoter from potato (WO 96/12814), the light-inducible PPDK promoter or the wounding-induced pinII promoter (EP375091).

Pathogen-inducible promoters comprise the promoters of genes which are induced as the result of a pathogen attack such as, for example, genes of PR proteins, SAR proteins, beta-1,3-glucanase, chitinase and the like (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968 (1989).

Also comprised are wounding-inducible promoters such as that of the promoter of the pinII gene (Ryan (1990) Ann Rev Phytopath 28:425-449; Duan et al. (1996) Nat Biotech 14:494-498), the promoters of the wun1 and wun2 genes (U.S. Pat. No. 5,428,148), the promoters of the win1 and win2 genes (Stanford et al. (1989) Mol Gen Genet. 215:200-208), of the systemin gene (McGurl et al. Science 225:1570-1573), the WIP1 gene (Rohmeier et al. (1993) Plant Mol Biol 22:783-792; Ekelkamp et al. (1993) FEBS Letters 323:73-76), or the MPI gene (Corderok et al. (1994) The Plant J 6(2):141-150) and the like.

Further suitable promoters are, for example, fruit-maturation-specific promoters such as the fruit-maturation-specific promoter from tomato (WO 94/21794, EP 409 625). Some of the development specific promoters are additionally tissue-specific since the individual tissues are formed as a function of the development.

Furthermore suitable are those promoters which ensure the expression in tissues or plant parts in which, for example, the biosynthesis of ketocarotenoids or their precursors takes place. Examples of preferred promoters are promoters with specificities for the anthers, ovaries, petals, sepals, flowers, leaves, stems and roots and combinations hereof.

Tuber-specific, storage-root-specific or root-specific promoters are, for example, the patatin promoter class I (B33) or the promoter of the cathepsin D inhibitor from potato.

Examples of leaf-specific promoters are, for example, the promoter of the cytosolic FBPase from potato (WO 97/05900), the SSU promoter (small subunit) of Rubisco (ribulose-1,5-bisphosphate carboxylase) or the ST-LSI promoter from potato (Stockhaus et al. (1989) EMBO J. 8:2445-2451).

Examples of anther-specific promoters are the 5126 promoter (U.S. Pat. No. 5,689,049, U.S. Pat. No. 5,689,051) or the glob-1 promoter or the g-zein promoter.

Promoters suitable for expression in plastids and/or chromoplasts are for example plastid derived promoters. A number of plastid functional promoters are available in the art. Such promoters include, but are not limited to the promoter of the D1 thylakoid membrane protein, psbA (Staub et al. (1993) EMBO Journal, 12(2):601-606) the 16s rRNA promoter region, Pm (Staub et al. (1992) Plant Cell 4:39-45) or the rbcL promoter from spinach.

Further promoters which are suitable for expression in plants are described in Rogers et al. (1987) Methods in Enzymol 153:253-277; Schardl et al. (1987) Gene 61:1-11 and Berger et al. (1989) Proc Natl Acad Sci USA 86:8402-8406.

Non-limiting flower specific promoters are disclosed in WO 04/27070, WO 05/019460, WO 06/117381 and EP06115339.1 as filed on Jun. 13, 2006.

Examples of flower-specific promoters are the phytoene synthase promoter (WO 92/16635), the promoter of the P-rr gene (WO 98/22593), the EPSPS promoter (database entry M37029), the DFR-A promoter (database entry X79723), the B gene promoter (WO 00/08920) and the CHRC promoter (WO 98/24300; Vishnevetsky et al. (1996) Plant J. 10, 1111-1118), and the promoters of the Arabidopsis gene loci At5g33370, At5g22430 and At1g26630.

Specific mention is made for the following promoters disclosed in WO05/019460:

| Name | Source |
|---|---|
| EPSPS Promoter | Petunia hybrida |
| B-Gene Promoter | Lycopersicon esculentum |
| PDS Promoter | Lycopersicon esculentum |
| CHRC Promoter | Cucumis sativus |

in WO 04/27070: the Arabidopsis promoters: P76, P60, P84
and in WO 06/117381: the Arabidopsis promoters: M1s, M2s, M3s, M1L, M2L

### f) Transit Peptides

Transit peptides are examples of targeting sequence. Targeting sequences ensure the subcellular localization in the apoplast, in the vacuole, in plastids, in the mitochondrium, in the endoplasmic reticulum (ER), in the nucleus, in oil bodies or other compartments.

The translocation in plastids, and a discussion of suitable transit peptides is described in:
Woolhead et al., Biochemical Society Transactions. 28 (Part4):491-494, 2000
Reumann et al., Molecular Membrane Biology. 22(1-2):73-NIL_{—}20, 2005
Lubeck et al., Physiologia Plantarum. 100(1):53-64, 1997 and
Robinson et al., Plant Molecular Biology. 38(1-2):209-221, 1998

### g) Expression in Plastids

The Ki-sequence or both the Ki-sequence and the Bc-sequence may be expressed in plastids such as chromoplasts, preferentially in flower chromoplasts. Such transplastomic plants are a further aspect of the present invention. Methods of plant plastid transformation and functional expression of genes in the organelles are known in the art. Such methods may be found for example in EP1458875 or EP1461439.

### h) Further Aspects of Proteins/Polypeptides/Enzymes of the Invention

The invention also comprises likewise “functional equivalents” of the specifically disclosed proteins/polypeptides/enzymes (subsequently simply referred to as polypeptides).

“Functional equivalents” or analogs of the specifically disclosed polypeptides are in the context of the present invention polypeptides which differ therefrom, such as, for example, those having a degree of homology of less than 100%, but which still have the desired biological activity.

“Functional equivalents” mean according to the invention in particular mutants, which have in at least one of the positions of the specific sequences described herein an amino acid which differs from that specifically mentioned, but nevertheless have one of the biological activities mentioned herein. “Functional equivalents” thus comprise the mutants obtainable by one or more amino acid additions, substitutions, deletions and/or inversions, it being possible for the changes to occur in any sequence position as long as they lead to a mutant having a property according to the invention. Functional equivalence exists in particular also when there is a qualitative agreement between the mutant and unmodified polypeptide in the reactivity pattern, i.e. for example identical biological effects are to be observed but differ greatly in the level of expression. Examples of suitable substitutions of amino acid residues are the following:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also precursors of the polypeptides described, and functional derivatives and salts of the polypeptides. The term “salts” means both salts of carboxyl groups and acid addition salts of amino groups of the protein molecules of the invention. Salts of carboxyl groups can be prepared in a manner known per se and comprise inorganic salts such as, for example, sodium, calcium, ammonium, iron or zinc salts, and salts with organic bases such as, for example, amines, such as triethanolamine, arginine, lysine or piperidine. Acid addition salts such as, for example, salts with mineral acids such as hydrochloric acid or sulfuric acid and salts with organic acids, such as acetic acid or oxalic acid are likewise an aspect of the invention.

“Functional derivatives” of polypeptides of the invention can likewise be prepared on functional amino acid side groups or on their N- or C-terminal end with the aid of known techniques. Derivatives of these types comprise for example aliphatic esters of carboxylic acid groups, amides of carboxylic acid groups, obtainable by reaction with ammonia or with a primary or secondary amine; N-acryl derivatives of free amino groups prepared by reaction with acyl groups; or O-acyl derivatives of free hydroxy groups prepared by reaction with acyl groups.

“Functional equivalents” of course also comprise polypeptides obtainable from other organisms, and naturally occurring variants. For example, areas of homologous sequence regions can be found by sequence comparison, and equivalent enzymes/polypeptides can be established on the basis of the specific requirements of the invention.

“Functional equivalents” are moreover fusion proteins having one of the abovementioned polypeptide sequences or functional equivalents derived therefrom, and at least one further heterologous sequence functionally different therefrom in functional N- or C-terminal linkage (i.e. with negligible mutual functional impairment of the portions of the fusion proteins). No limiting examples of such heterologous sequences are other enzymes.

“Functional equivalents” also comprised by the invention are homologues of the specifically disclosed proteins. These have at least 60%, preferably at least 75%, in particular at least 85%, such as, for example, 90%, 95, 96, 97, 98 or 99%, homology to one of the specifically disclosed sequences, calculated by the algorithm of Pearson and Lipman, Proc. Natl. Acad, Sci. (USA) 85(8), 1988, 2444-2448. A percentage homology of a homologous polypeptide of the invention means in particular the percentage identity of the amino acid residue based on the complete length of one of the amino acid sequences specifically described herein.

A “derived” amino acid sequence means according to the invention, unless indicated otherwise, a sequence which has an identity of at least 80% or at least 90%, in particular 91%, 92%, 93%, 94%, 95%, 96%, 97%, 98% and 99%, with the initial sequence.

“Identity” or “homology” between two sequences means identity of the amino acid residues over the complete length of the sequence in each case, such as, for example, the identity calculated by comparison with the aid of the Vector NTI Suite 7.1 Software from Informax (USA) using the Clustal method (Higgins D G, Sharp P M. Fast and sensitive multiple sequence alignments on a microcomputer. Comput Appl. Biosci. 1989 April; 5(2):151-1), setting the following parameters:

### Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

In the case where protein glycosylation is possible, equivalents of the invention comprise proteins of the type designated above in deglycosylated or glycosylated form and modified forms obtainable by altering the glycosylation pattern.

Homologues of the peptides of the invention can be identified by screening combinatorial libraries of mutants such as, truncation mutants. For example, it is possible to generate a variegated library of peptide variants by combinatorial mutagenesis at the nucleic acid level, for example, by enzymatic ligation of a mixture of synthetic oligonucleotides. There are a large number of methods which can be used to produce libraries of potential homologues from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide all sequences which encode the desired set of potential protein sequences in one mixture. Methods for synthesizing degenerate oligonucleotides are known to the skilled worker (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

### i) Further Aspects on Nucleic Acids

All nucleic acid sequences of the invention (single- and double-stranded DNA and RNA sequences, such as cDNA or mRNA) can be prepared in a manner known per se by chemical synthesis from the nucleotide units, for example, by fragment condensation of individual overlapping, complementary nucleic acid units of the double helix. Chemical synthesis of oligonucleotides can take place for example in a known manner, for example by the phosphoamidite method (Voet, Voet, 2nd edition, Wiley Press New York, pages 896-897). Addition of synthetic oligonucleotides and filling in of gaps using the Klenow fragment of DNA polymerase and ligation reactions, and general cloning methods are for instance described in Sambrook et al. (1989), Molecular Cloning: A laboratory manual, Cold Spring Harbor Laboratory Press.

A “derived” nucleic acid sequence means according to the invention, unless indicated otherwise, a sequence which has an identity of at least 80% or at least 90%, in particular 91%, 92%, 93%, 94%, 95%, 96%, 97%, 98% and 99%, with the initial sequence.

“Identity” or “homology” between two nucleic acids means the identity of the nucleotides over the complete length of the nucleic acid in each case, in particular the identity by comparison with the aid of the Vector NTI Suite 7.1 Software from Informax (USA) using the Clustal method (see above).

The invention also relates to nucleic acid sequences coding for one of the above peptides and their functional equivalents, which can be obtained for example by use of artificial nucleotide analogs.

The invention relates both to isolated nucleic acid molecules which code for peptides of the invention or biologically active segments thereof, and nucleic acid fragments which can be used for example as hybridization probes or primers for identifying or amplifying coding nucleic acids of the invention.

The nucleic acid molecules of the invention may additionally comprise untranslated sequences from the 3′ and/or 5′ end of the coding region of the gene.

“Isolated” nucleic acid molecules are separated from other nucleic acid molecules which are present in the natural source of the isolated nucleic acid and may moreover be substantially free of other cellular material or culture medium if it is prepared by recombinant techniques, or free of chemical precursors or other chemicals if it is synthesized chemically.

A nucleic acid molecule of the invention can be isolated by means of standard techniques of molecular biology and the sequence information provided by the invention. For example, cDNA can be isolated from a suitable cDNA library by using one of the specifically disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). It is moreover possible to isolate a nucleic acid molecule comprising one of the sequences of the invention or a segment thereof by polymerase chain reaction using the oligonucleotide primers constructed on the basis of this sequence. The nucleic acid amplified in this way can be cloned into a suitable vector and characterized by DNA sequence analysis. The oligonucleotides of the invention can also be prepared by standard synthesis methods, e.g. using an automatic DNA synthesizer.

The invention further comprises the nucleic acid molecules complementary to the specifically described nucleotide sequences, or a segment thereof.

The nucleotide sequences of the invention make it possible to produce probes and primers which can be used for identifying and/or cloning homologous sequences in other cell types and organisms. Such probes and primers usually comprise a nucleotide sequence region which hybridizes under stringent conditions to at least about 12, preferably at least about 25, such as, for example, about 40, 50 or 75, consecutive nucleotides of a sense strand of a nucleic acid sequence of the invention or of a corresponding antisense strand.

Further nucleic acid sequences of the invention are derived from the sequences as specifically mentioned herein and differ therefrom by addition, substitution, insertion or deletion of one or more nucleotides, but still code for peptides having the desired profile of properties.

The invention also comprises nucleic acid sequences, which comprise so-called silent mutations, as well as naturally occurring variants such as, for example, splice variants or allelic variants, thereof. Sequences obtainable by conservative nucleotide substitutions (i.e. the relevant amino acid is replaced by an amino acid of the same charge, size, polarity and/or solubility) are likewise an aspect.

The invention also relates to the molecules derived from the specifically disclosed nucleic acids through sequence polymorphisms. These genetic polymorphisms may exist because of the natural variation between individuals within a population. These natural variations normally result in a variance of from 1% to 5% in the nucleotide sequence of a gene.

The invention further also comprises nucleic acid sequences which hybridize with the abovementioned coding sequences or are complementary thereto. These polynucleotides can be found by screening genomic or cDNA libraries and if appropriate be amplified therefrom by means of PCR with suitable primers, and subsequently isolated for example with suitable probes. A further possibility is the transformation of suitable microorganisms with polynucleotides or vectors of the invention, to multiply the microorganisms and thus the polynucleotides and subsequently to isolate them. An additional possibility is to synthesize polynucleotides of the invention also by a chemical route.

The property of being able to “hybridize” onto polynucleotides means the ability of a polynucleotide or oligonucleotide to bind under stringent conditions to an almost complementary sequence, while there are nonspecific bindings between non-complementary partners under these conditions. For this purpose, the sequences should be from 70% to 100%, preferably from 90% to 100%, complementary. The property of complementary sequences being able to bind specifically to one another is made use of, for example, in the Northern or Southern blotting technique or in the primer binding in PCR or RT-PCR. Oligonucleotides with a length of 30 base pairs or more are normally employed for this purpose. Stringent conditions mean, for example, in the Northern blotting technique the use of a washing solution, for example 0.1×SSC buffer with 0.1% SDS (20×SSC: 3M NaCl, 0.3M Na citrate, pH 7.0), from 50° C. to 70° C., preferably from 60° C. to 65° C., for eluting nonspecifically hybridized cDNA probes or oligonucleotides. In this case, as mentioned above, only nucleic acids with a high degree of complementarity remain bound to one another. The setting up of stringent conditions is known to the skilled worker and is described for example in Ausubel et al., Current Protocols in Molecular Biology, John Wiley & Sons, N.Y. (1989), 6.3.1-6.3.6.

For example, the conditions during the washing step can be selected from the range of conditions delimited by those with less stringency (with 2×SSC at 50° C.) and those with high stringency (with 0.2×SSC at 50° C., preferably at 65° C.) (20×SSC: 0.3 M sodium citrate, 3 M sodium chloride, pH 7.0).

Moreover, the temperature during the washing step can be increased from moderate conditions at room temperature, 22° C., to stringent conditions at 65° C.

Both parameters, salt concentration and temperature can be varied simultaneously, or else one of the two parameters can be kept constant, while only the other one is varied. Also, denaturing agents such as, for example, formamide or SDS can be employed during the hybridization step. In the presence of 50% formamide, the hybridization is preferably carried out at 42° C.

Some examples of conditions for hybridization and washing step are shown herein below:
(1) hybridization conditions with, for example,
(i) 4×SSC at 65° C., or [0074] (ii) 6×SSC at 45° C., or
(iii) 6×SSC at 68° C., 100 mg/ml denatured fish sperm DNA, or
(iv) 6×SSC, 0.5% SDS, 100 mg/ml denatured, fragmented salmon sperm DNA at 68° C., or
(v) 6×SSC, 0.5% SDS, 100 mg/ml denatured, fragmented salmon sperm DNA, 50% formamide at 42° C., or
(vi) 50% formamide, 4×SSC at 42° C., or
(vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
(viii)2.times.or 4×SSC at 50° C. (moderate conditions), or
(ix) 30 to 40% formamide, 2.times.or 4×SSC at 42° C. (moderate conditions).
(2) washing steps for in each case 2×20 minutes, with, for example,
(i) 0.015 M NaCl/0.0015 M sodium citrate/0.1% SDS at 50° C., or
(ii) 0.1×SSC at 65° C., or
(iii) 0.1×SSC, 0.5% SDS at 68° C., or
(iv) 0.1×SSC, 0.5% SDS, 50% formamide at 42° C., or
(v) 0.2×SSC, 0.1% SDS at 42° C., or
(vi) 2×SSC at 65° C. (moderate conditions).

### j) Expression Constructs and Vectors

The invention additionally relates to expression constructs comprising, under the genetic control of regulatory nucleic acid sequences, a nucleic acid sequence coding at least one polypeptide of the invention and to vectors comprising at least one of these expression constructs.

Such constructs of the invention preferably comprise a promoter 5′-upstream from the particular coding sequence, and a terminator sequence 3′-downstream, and, if appropriate, other usual regulatory elements, in particular each operatively linked to the coding sequence.

“Transcription” is understood according to the invention as meaning the process by which, starting from a DNA matrix, a complementary RNA molecule is prepared. Proteins such as RNA polymerase, “sigma factors” and transcriptional regulator proteins are involved in this process. The RNA synthesized is then used as a matrix in the translation process, which then leads to the biosynthetically active protein.

“Operative linkage” or “functional linkage” means the sequential arrangement of promoter, coding sequence, terminator and, if appropriate, other regulatory elements in such a way that each of the regulatory elements is able to comply with its function as intended for expression of the coding sequence. To this end, a direct linkage in the chemical sense is not imperative. Genetic control sequences, such as enhancer sequences, can also exert their function on the target sequence from positions which are further removed or even from other DNA molecules. Arrangements are preferred in which the nucleic acid sequence to be expressed or the gene to be expressed is positioned behind (i.e. at the 3′-end) the promoter sequence according to the invention, such that both sequences are bonded covalently to one another. Preferably, the distance between the promoter sequence and the nucleic acid sequence to be expressed is in this case lower than 200 base pairs, particularly preferably less than 100 base pairs, very particularly preferably less than 50 base pairs. Examples of sequences which can be operatively linked are targeting sequences and enhancers, polyadenylation signals. Other regulatory elements comprise amplification signals, origins of replication and translation enhancers such as the tobacco mosaic virus 5′-leader sequence (Gallie et al., Nucl. Acids Res. 15 (1987), 8693-8711). Suitable regulatory sequences are described, for example, in Goeddel, Gene Expression Technology: Methods in Enzymology 185, Academic Press, San Diego, Calif. (1990).

In addition to the artificial regulatory sequences it is possible for the natural regulatory sequence still to be present in front (i.e. at the 5′-end) of the actual structural gene. This natural regulation can, if appropriate, be switched off by genetic modification, and expression of the genes can be increased or decreased. The gene construct can, however, also have a simpler structure, that is to say no additional regulatory signals are inserted in front of the structural gene, and the natural promoter with its regulation is not deleted. Instead, the natural regulatory sequence is mutated so that regulation no longer takes place, and gene expression is enhanced. The nucleic acid sequences may be present in one or more copies in the gene construct.

The regulatory sequences are intended to make specific expression of the nucleic acid sequences and protein expression possible. This may mean, for example, depending on the host organism, that the gene is expressed or over expressed only after induction or that it is immediately expressed and/or over expressed.

The regulatory sequences or factors may moreover preferably influence positively, and thus increase expression. Thus, enhancement of the regulatory elements can take place advantageously at the level of transcription by using strong transcription signals such as promoters and/or enhancers. However, it is also possible to enhance translation by, for example, improving the stability of the mRNA.

An expression cassette is produced by fusing a suitable promoter to a suitable coding nucleotide sequence and to a terminator signal or polyadenylation signal. Conventional techniques of recombination and cloning are used for this purpose, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

An expression cassette or a vector comprising a suitable promoter, suitable coding nucleotide sequence and a terminator signal or polyadenylation signal can also be produced by synthesis of the entire sequence as described above.

Examples of suitable expression vectors which may be mentioned are:

Suitable plant expression vectors are for example described in detail in: Becker, D., Kemper, E., Schell, J. and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721.

### k) Transfer of Foreign Genes into a Plant

The transfer of foreign genes in the genome of plants is referred to as transformation.

To this end, it is possible to exploit methods which are known per se for the transformation and regeneration of plants from plant tissues or plant cells in order to carry out a transient or stable transformation.

Suitable methods for the transformation of plants are the transformation of protoplasts by means of polyethylene-glycol-induced DNA uptake, the biolistic method using the gene gun, known as “particle bombardment method”, electroporation, incubation of dry embryos in DNA-comprising solution, microinjection, and Agrobacterium-mediated gene transfer. The above methods are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225).

By preference, the construct to be expressed is cloned into a vector which is suitable for the transformation of Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria which have been transformed with an expression plasmid can be used in the known manner for the transformation of plants, for example by bathing scarified leaves or leaf segments in an agrobacterial solution and subsequently growing them in suitable media.

For the preferred generation of genetically modified plants, herein below also referred to as transgenic plants, the fused expression cassette which expresses a ketolase is cloned into a vector, for example pBin19 or pSUN2, which is suitable for being transformed into Agrobacterium tumefaciens. Agrobacteria which have been transformed with such a vector can then be used in the known manner for the transformation of plants, in particular crop plants, for example by bathing scarified leaves or leaf segments in an agrobacterial solution and subsequently growing them in suitable media.

Suitable Agrobacteria are for example Agrobacterium tumefaciens or Agrobacterium rhizogenes. Other Agrobacteria useful for plant transformation are known in the art and can be used in the process of the present invention.

The transformation of plants by Agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants can be regenerated in the known manner from the transformed cells of the scarified leaves or leaf segments or hypocotyls, and such plants comprise a gene for the expression of a nucleic acid encoding a ketolase integrated into the expression cassette.

To transform host plants with a nucleic acid which encodes a ketolase, an expression cassette is incorporated, as insertion, into a recombinant vector whose vector DNA comprises additional functional regulatory signals, for example sequences for replication or integration. Suitable vectors are described, inter alia, in “Methods in Plant Molecular Biology and Biotechnology” (CRC Press), chapter 6/7, pp. 71-119 (1993).

### l) Preparation of Transgenic Ketocarotenoid Producing Tagetes Plants

Recombinant Tagetes plants expressing expression improved ketolase activity or both improved ketolase activity and beta-cyclase activity may be prepared starting from per se known ketolase-encoding sequences (see Annex 1) or beta-cyclase-encoding sequences (Annex 2) and improving the sequence of the respective enzyme by for example adapting the sequence to the codon usage of the Tagetes plants to be used. Suitable Tagetes plants may be transformed in a manner known per se with the modified ketolase coding sequence or both the modified ketolase coding sequence and the beta-cyclase coding sequence. Specific examples are given in the attached experimental part. Based on the specific information a skilled reader will be in a position, to prepare transformants of different Tagetes plants modified by the same or different ketolase coding sequences.

Suitable methods of preparing transgenic Tagetes plants are disclosed in EP-A-1 240 342.

### m) Carotenoid Extraction Methods

Carotenoids and their esters, such as astaxanthin and its mono- and diesters, can be extracted from the carotenoid-containing plants or plant parts, which have previously been dried and/or comminuted where appropriate, by organic solvents such as, for example, by acetone, hexane, methylene chloride, tert-butyl methyl ether or by solvent mixtures such as ethanol/hexane or acetone/hexane. The extractive effect can be varied on the basis of differences in polarity through different solvent mixing ratios. Enrichment of carotenoids and their esters to high concentration is possible by such an extraction.

Extracts prepared in this way are particularly suitable as reactant for carrying out the chemical hydrolysis reaction of the invention.

### n) Workup of the Ester Hydrolysis Products

The carotenoids, especially ketocarotenoids as obtained by hydrolysis, can advantageously be isolated from the aqueous reaction solution by extraction. The extraction can be repeated more than once to increase the yield. Examples of suitable extractants are organic solvents such as toluene, methylene chloride, butyl acetate, diisopropyl ether, benzene, MTBE (Methyl-tert-butylether), petroleum ether or ethyl acetate.

After concentration of the organic phase obtained in this way, the products can ordinarily be isolated in good chemical purities.

The identity and purity of the isolated compound(s) can be determined by known techniques. These include high performance liquid chromatography (HPLC), spectroscopic methods, staining methods, thin-layer chromatography, enzyme assay or microbiological assays. These analytical methods are summarized in: Patek et al. (1994) Appl. Environ. Microbiol. 60:133-140; Malakhova et al. (1996) Biotekhnologiya 11 27-32; and Schmidt et al. (1998) Bioprocess Engineer. 19:67-70. Ulmann's Encyclopedia of Industrial Chemistry (1996) Vol. A27, VCH: Weinheim, pages 89-90, 521-540, 540-547, 559-566, 575-581 and 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

### o) Applications of the Products Prepared According to the Invention

The carotenoids obtained according to the invention are particularly suitable as additives for human and animal foods. As additive for human food, they may be used for coloring of any food product for example beverages, sweets or convenient food. In case they are used as animal food additive they promote in particular pigmentation after, preferably oral, administration.

“Pigmentation” means according to the invention preferably the intensification or causation of a color of at least part of an animal or animal product of the pigmented animal compared with the non-pigmented animal. Thus, in particular, astaxanthin-containing pigmenting agents generate or intensify a pink to pinkish red hue.

Preferred animals, which can be pigmented by the oral administration of the invention are animals selected from fish, crustaceans or birds, especially galliformes and anatidae. Preferred fish are salmonids, especially salmon or trout. Preferred crustaceans are shrimps or crayfish. Preferred galliformes are chickens, ducks and geese. Preferred anatidae are flamingo.

Depending on the pigmented animal, the preferred pigmented animal products mean, in particular, flesh for salmon or trout, skin for chickens, ducks or geese, feathers for chickens, ducks, geese or flamingo and egg or yolk for chickens, ducks or geese.

Oral administration of the carotenoids to animals can take place directly or, preferably, by oral administration of animal food preparations previously admixed with the carotenoid. The carotenoids may in this case be in liquid or solid form.

The carotenoids may, as long as the solvents still present are physiologically harmless for the appropriate animals, be added directly to the animal food preparation or be employed in the form of carotenoid-containing powders or oils after evaporation of the solvents still present. Previous purification of the resulting hydrolysis product is not absolutely necessary.

The resulting carotenoid-containing powders or oils can for example be incorporated in fish oil, be applied to powdered carrier materials such as, for example, wheat flour, or be enclosed in alginates, gelatin or lipids.

The invention also relates to animal food preparations comprising at least one carotenoid hydrolysate of the invention in addition to conventional animal food ingredients.

Thus, for example, a fish food preparation may comprise further conventional fish food components such as, for example, fish meal and/or other proteins, oils such as, for example, fish oils, cereals, vitamins, minerals, preservatives and, where appropriate, medicaments in conventional amounts.

A typical fish food formula for trout is composed for example of the following components:

| | % | Weight for 500 kg |
|---|---|---|
| Components | by weight | kg |
| Fish meal | 30.00 | 150.00 |
| Full-fat soybeans | 20.00 | 100.00 |
| Pregelatinized wheat flour | 18.00 | 90.00 |
| Vitamin premix | 0.80 | 4.00 |
| Choline chloride (50%) | 0.20 | 1.00 |
| Wheat gluten | 20.00 | 100.00 |
| Sipernat 50S | 3.00 | 15.00 |
| Fish oil | 8.00 | 40.00 |

A typical fish food formula for salmon is composed for example of the following components:

| Components | % by weight |
|---|---|
| Fish meal | 75.00 |
| Vegetable protein | 5.00 |
| Cereals | 7.80 |
| Vitamins/minerals | 1.00 |
| Antioxidants/preservatives | 0.20 |
| Fish oil | 11.00 |

The carotenoids of the invention can be admixed in the form of powder or liquid for example solved or suspended in oil to the animal food preparations. The animal food preparations obtained in this way can be pelleted or, particularly advantageously, extruded in a manner known per se.

In a preferred embodiment, the carotenoid-containing products are admixed preferably in the liquid form to the animal food preparations. This is particularly advantageous for producing extruded feed preparations. The extrusion process may lead to extrusion stress on the sensitive substances such as, for example, astaxanthin, which may lead to loss of astaxanthin. Extrusion stress takes the form primarily of the action of mechanical forces (kneading, shearing, pressure, etc.) but also of hydro-thermal stress caused by addition of water and water vapor; oxidative stress is also to be observed.

In order to avoid the losses of substance occurring in the extrusion process described above, it is possible to apply liquid carotenoid-containing extracts by the so-called PPA (post pelleting application) technique after the extrusion and drying process under vacuum.

The carotenoids may also be administered orally to animals directly as long as the solvents still present are physiologically harmless to the corresponding animals.

However, the carotenoids can also be administered in the form of powders or oils only after evaporation of the solvents still present.

The resulting carotenoid-containing powders or oils can for example be incorporated in fish oil, be applied to powdered carrier materials such as, for example, wheat flour, or be enclosed in alginates, gelatin or lipids.

The invention therefore also relates to pigmenting agents comprising carotenoids produced by the invention.

The invention is illustrated by the following examples but is not restricted to these:

### EXPERIMENTAL PART

### Example 1

### Tagetes germplasm

### Generation of Lutein-Depleted Tagetes

Naturally occurring Tagetes plants which accumulate relatively high concentrations of beta-carotenoids while most of the alpha-carotenoids do not accumulate are not known. Therefore, it was the task to develop a Tagetes plant which fulfills the requirements of I) being largely devoid of lutein and associated alpha-carotenoids in its flower petals and ii) being characterized with relatively high levels of total carotenoids.

The process for creating Tagetes plants with lutein-depleted flowers is described in the U.S. Pat. No. 6,784,351. This patent describes in detail i) the EMS mutagenesis of Tagetes erecta “Scarletade” and “13819”, and ii) the HPLC screening procedure to identify certain abnormal carotenoid profiles in flowers of “Scarletade” and “13819”. The especially interesting mutant of Scarletade, 124-257, is described by its changed carotenoid profile in petals and leaves.

### Breeding of 31360-2-08, 31360-2-09 and Hybrid Line 31360-2-09-8-8ApxM

Tagetes erecta selection 124-257, described in U.S. Pat. No. 6,784,351, was found to have a low transformation rate using the identified tissue culture regeneration medium and Agrobacterium transformation technique. Using a standardized method, different plant selections can be transformed at different rates; therefore, to recover a target number of transformed plants, it can be expected that a selection having a low transformation rate would require use of a higher number of explants than a selection having a high transformation rate. A selection having a low transformation rate would require at least about 200 explants to recover about 1 transformed plant.

Instead of further optimizing the transformation protocol, a plant breeding backcross technique well known to those skilled in the art was used to transfer the mutation resulting in the increased zeaxanthin to lutein ratio of selection 124-257 to a selection having a higher transformation rate. Several Tagetes erecta marigold plants were identified as having acceptable transformation rates, and from these Tagetes erecta marigold plant named 13819 was selected. Tagetes erecta 13819 is a proprietary breeding selection of PanAmerican Seed located at 622 Town Road, West Chicago, Ill. 60185.

In the backcross program, selection 124-257 was used as the female parent in a cross with a selection of 13819 as the male parent. The resulting population was identified as 11754, and from this population a plant identified as 11754-2F was selected based on its hybrid characteristics. Plant 11754-2F was selfed and from this population plant identified as 11754-2F-1 was selected based on carotenoid profile and plant habit. Plant 11754-2F-1 was used as male parent in a cross with 13819 as the female parent. The resulting population was identified as 31360, and from this population a plant identified as 31360-2 was selected based on carotenoid profile and plant habit. Plant 31360-2 was selfed and from the resulting population, plants 31360-2-08 and 31360-2-09 were selected based on carotenoid profile, total carotenoid concentration, and plant habit. Both selections were selfed and seed from the cross was used to test transformation rates, and the seedlings from both selections were found to have acceptable transformation rates. In addition, the resulting plants from the selfed 31360-2-08 plant were found to be uniform for carotenoid profile, carotenoid concentration, and plant habit. The resulting plants from the selfed 31360-2-09 plant were found to segregate for total carotenoid concentration and plant habit characteristics. From the selfed 31360-2-09 population, a plant identified as 31360-2-09-08 was selected based on carotenoid profile, total carotenoid concentration, and plant habit. The selfed population from the 31360-2-09-08 plant was found to be uniform for carotenoid profile, carotenoid concentration, and to have an acceptable transformation rate. Plant 31360-2-09-08 was selfed, and from the selfed population, a plant identified as 31360-2-09-08-08 was selected based on flower morphology, carotenoid profile, total carotenoid concentration and plant habit. In addition, from the selfed population, plants identified as 31360-2-09-08-01, 31360-2-09-08-02, 31360-2-09-08-04, 31360-2-09-08-05, 31360-2-09-08-09, 31360-2-09-08-011 and 31360-2-09-08-12 were selected based on flower morphology, carotenoid profile, total carotenoid concentration and plant habit. Line 31360-2-09-8-8ApxM was created by crossing, using the progeny of 31360-2-09-08-08 as the male parent and the progeny of 31360-2-09-08-01, 31360-2-09-08-02, 31360-2-09-08-04, 31360-2-09-08-05, 31360-2-09-08-09, 31360-2-09-08-011 and 31360-2-09-08-12 as the female parents. Seed from these crosses were pooled and used for transformation.

### Example 2

### Tagetes Transformation Protocol for 31360-2-08, 31360-2-09 and 31360-2-09-8-8ApxM

Seeds of Tagetes erecta line 31360-2-08, 31360-2-09 and 31360-2-09-8-8ApxM were disinfected with 2% NaOCl solution for 10 minutes followed by three washes with autoclaved distilled water. Afterwards seeds were dried and can be stored under aseptic conditions at room temperature for a period of up to two weeks before in vitro germination. Germination occurred on solidified MS medium (Murashige, T., and Skoog, F., A revised medium for rapid growth and bioassays with tobacco tissue cultures. Physiol. Plant. 15, 473-497, 1962) in a 16/8 h light/darkness photoperiod for 1-3 weeks. Cotyledonary segments were prepared and used as primary target material for transformation. These segments were inoculated for 20 minutes in liquid MS medium containing Agrobacterium tumefaciens strain EHA105 cells at an OD600 of 0.1. The binary vector contained the gene pat (plus additional gene cassettes encoding effect genes) and allows therefore phosphinothricin (PPT) and/or BASTA selection. Explants are co-cultured for a period of 6 days on MS medium (pH 5.8) solidified with 0.8% agar and supplemented with 1 mg/l 3-indole-3-acetic acid (IAA), 3 mg/l indole-3-butyric acid (IBA), 500 mg/l 2-(N-morpholino) ethanesulfonic acid (MES) and 2% sucrose. Cultivation occurred under controlled conditions at 21° C., 35-40 μmol m⁻² s⁻¹ white light intensity and 16/8 light/darkness rhythm. Shoots were induced on cotyledon explants on fresh MS medium, adjusted to pH 5.5, as described before supplemented with 500 mg/l Timentin, 1 mg/l PPT, 5 mg/l Silver Nitrate (AgNO₃). Second to fourth subcultures were done onto fresh MS medium following the formulation described above at pH 5.8 and 15 days subculture period. The newly formed shoot buds were transferred to a new medium to promote shoot regeneration. The shoot regeneration medium follows the formulation of MS supplemented with 0.7% agar, 250 mg/l Timentin, 1 mg/l PPT, 5 mg/l AgNO₃, 1 mg/l IAA, 3 mg/l 6-Benzylaminopurine (BAP), 500 mg/l MES and 2% sucrose and was adjusted to pH 5.8. Three subcultures were promoted in a 15 days subculture period. Regenerated shoots were then transferred onto elongation medium (MS) supplemented with 0.7% agar, 250 mg/l Timentin, 1 mg/l PPT, 5 mg/l AgNO₃, 0.5 mg/l IAA, 0.5 mg/l gibberellic acid (GA3), 500 mg/l MES, 2% sucrose, pH 5.8. Three subcultures were performed, each for 15 days. Well elongated shoots (1.5-3.5 cm in length) with well expanded leaves were transferred onto rooting MS medium solidified with 0.7% agar and supplemented with 250 mg/l Timentin, 1 mg/l PPT, 0.5 mg/l IBA, 500 mg/l MES, 2% sucrose and adjusted to pH 5.8. Leaf material from rooted plants was analyzed by qPCR for the selection marker gene in order to confirm transgenicity and to determine the copy number of the construct integrated into the genome. After four weeks the well rooted transgenic shoots were transferred to ex vitro-conditions at the greenhouse. Hardening of plants in soil could be achieved with inverted funnels. They prevented dehydration of the plantlets. Afterwards plants were transferred into bigger pots with soil to promote growth and development until flowering under greenhouse conditions.

### Example 3

### Biochemical Analytics Protocol

10-20 mg fresh material of Tagetes petals were homogenized (via mortar and pestle in liquid nitrogen). The homogenous material was extracted with acetone, usually three times with 500ul acetone till the supernatant is colorless. If needed, material was shaken after each extraction. All supernatants were combined and evaporated to dryness using a speedvac concentrator.

### Xanthophyll Analysis

The pellet was dissolved in 180 μl of acetone and eventually briefly sonicated. For saponification, 20 μl of 10% KOH (in methanol) was added and incubated for 30 minutes under constant shaking (1000 rpm) in the dark at room temperature. The reaction was stopped by the addition of 20-30 μl 1M HCl (till neutral pH value was reached). Samples were centrifuged for 10 min at 13.000 rpm to pellet debris and analyzed by HPLC.

### Ketocarotenoid Analysis

The concentrated carotenoids of 5 mg dry petal material were transferred to an anaerobic gloves box (e.g. manufacturer COY Laboratory Products Inc., USA) which allow chemical reactions at very low oxygen levels (around 1-10 ppm). Inside the glove box, the pellet was dissolved in 200 μl toluene. 200 μl of fresh 0.5 M sodium methoxide was added, the solutions were thoroughly mixed, and reaction proceeded for 10 min at 9° C. (or lower) at constant shaking at 1000 rpm. The reaction was stopped by adding 200 μl 0.5 M sulfuric acid to neutralize the reaction. Perhaps, more sulfuric acid needs to be added for neutralization. Reaction vials were taken out of the anaerobic chamber, briefly centrifuged, and carotenoids were extracted with toluene (about 5 times with 200 μl). The combined toluene extracts were combined and evaporated to dryness. Carotenoids were re-suspended in small volume for HPLC analysis.

The analysis of samples prepared according to the procedure described above was done under the following HPLC conditions:
HPLC column: Prontosil C30, 250×4.6 mm, (Bischoff, Leonberg, Germany)
Flow rate: 1.0 ml/min
Eluents: Solvent A—100% methanol Solvent B—80% methanol, 0.2% ammonia acetate Solvent C—100% t-butyl-methylether
Detection: 300-530 nm

### Gradient Profiles:

| Time (min) | Flow rate | % Solvent A | % Solvent B | % Solvent C |
|---|---|---|---|---|
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids were: violaxanthin at about 11, 7 min, zeaxanthin at about 21 min and beta-carotene at 32 min.

### Materials and General Methods

Unless indicated otherwise, chemicals and reagents in the Examples were obtained from Sigma Chemical Company (St. Louis, Mo.), restriction endonucleases were from New England Biolabs (Beverly, Mass.) or Roche (Indianapolis, Ind.), oligonucleotides were synthesized by MWG Biotech Inc. (High Point, N.C.), and other modifying enzymes or kits regarding biochemicals and molecular biological assays were from Clontech (Palo Alto, Calif.), Pharmacia Biotech (Piscataway, N.J.), Promega Corporation (Madison, Wis.), or Stratagene (La Jolla, Calif.). Materials for cell culture media were obtained from Gibco/BRL (Gaithersburg, Md.) or DIFCO (Detroit, Mich.). The cloning steps carried out for the purposes of the present invention, such as, for example, restriction cleavages, agarose gel electrophoresis, purification of DNA fragments, transfer of nucleic acids to nitrocellulose and nylon membranes, linking DNA fragments, transformation of E. coli cells, growing bacteria, multiplying phages and sequence analysis of recombinant DNA, are carried out as described by Sambrook (1989). The sequencing of recombinant DNA molecules is carried out using ABI laser fluorescence DNA sequencer following the method of Sanger (Sanger 1977).

### Example 4

### Cloning of the Fragment Encoding the Tomato B-Gene Lycopene Beta-Cyclase

To isolate the fragment described by SEQ ID NO: 15, RNA is isolated from mature flower petals of Lycopersicum esculentum according to published methods (e.g. Maniatis T, Fritsch E F, and Sambrook J Molecular Cloning: A Laboratory Manual, Cold Spring Harbor Laboratory, Cold Spring Harbor (NY), (1989); Qiagen, RNeasy Mini Handbook 06/2001). The isolated RNA is employed as matrix for cDNA synthesis according to published methods (e.g. Maniatis T, Fritsch E F, and Sambrook J Molecular Cloning: A Laboratory Manual, Cold Spring Harbor Laboratory, Cold Spring Harbor (NY), (1989)). The resulting cDNA is employed as matrix DNA for a polymerase chain reaction (PCR) mediated amplification using the oligonucleotide primers SEQ ID NO 18 and SEQ ID NO 19. Alternatively, the fragment described by SEQ ID NO: 15 can be generated by in vitro synthesis.

| | | |
|---|---|---|
| SEQ ID NO 18: | PR369 | cccgggatggaagctcttctcaa |
| SEQ ID NO 19: | PR370 | ctgcagtcacattcaaaggctctctatt |

SEQ ID NO 15: tomato B-Gene coding sequence
Position 1 to 24 Primer binding region for primer PR369
Position 1488o 1515 Primer binding region for primer PR370
Position 7 to 1500 coding sequence of B-gene from tomato

### Example 5

### Cloning of the Coding Sequence of the Beta-Carotene Ketolase from Scenedesmus Vacuolatus Strain 211-8B from the Culture Collection of the University of Goettingen (SAG)

To isolate the DNA fragment described by SEQ ID NO: 20, Scenedesmus vacuolatus SAG211-8b was grown as for 14 days under low light conditions in basal media with peptone, as recommended by the culture collection of the University of Goettingen (SAG). RNA was isolated from tissue of Scenedesmus vacuolatus according to published methods (e.g. Maniatis T, Fritsch E F, and Sambrook J Molecular Cloning: A Laboratory Manual, Cold Spring Harbor Laboratory, Cold Spring Harbor (NY), (1989); Qiagen, RNeasy Mini Handbook 06/2001). The isolated RNA was employed as matrix for cDNA synthesis according to published methods (e.g. Maniatis T, Fritsch E F, and Sambrook J Molecular Cloning: A Laboratory Manual, Cold Spring Harbor Laboratory, Cold Spring Harbor (NY), (1989)). The resulting cDNA was employed as matrix DNA for a polymerase chain reaction (PCR) mediated amplification using the oligonucleotide primers SEQ ID NO: 21 and SEQ ID NO: 22. Alternatively, the fragment described by SEQ ID NO: 20 can be generated by in vitro synthesis.

| | | |
|---|---|---|
| SEQ ID NO 21: | SVK-10 | gcgcatatggctcccaggcggcaa |
| SEQ ID NO 22: | SVK-11 | CGGTCGACTTACTCCACTACTGCTCC |

SEQ ID NO 20: Scenesdesmus vacuolatus beta-carotene ketolase coding sequence
Position 1 to 24 Primer binding region for primer SVK-10
Position 985 to 1010 Primer binding region for primer SVK-11
Position 7 to 1002 coding sequence of beta-carotene ketolase from Scenesdesmus vacuolatus
SEQ ID NO 23: Scenesdesmus vacuolatus beta-carotene ketolase protein sequence

### Example 6

### Expression of the Ketolase Gene from Haematococcus pluvialis Optimized for Expression in Tagetes erecta

### Determination of Tagetes Codon Usage

A cDNA library was created from Tagetes petals (cv. Scarletade) using state-of-the art technology (e.g. Chenchik et al., Clontechniques X(1):5-8, 1995; A Laboratory guide to RNA: Isolation, Analysis and Synthesis, edited by P. A:Krieg (Wiley-Liss. Inc, 1996; or as described in the instruction manuals of companies like Clontech, Invitrogen). A collection of several thousand ESTs (11118 clones) was sequenced and contig sequences generated. These sequences were the basis for generation of a codon-usage table which included 778346 good codons and 4693 good ORFs.

Codon Usage Table for Tagetes erecta:

A (Alanine):
GCA: 32.934%
GCU: 41.017%
GCG: 10.628%
GCC: 15.419%
C (Cysteine):
UGU: 61.935%
UGC: 38.064%
D (Aspartate):
GAU: 71.329%
GAC: 28.67%
E (Glutamate):
GAA: 60.0%
GAG: 40.0%
F (Phenylalanine):
UUU: 64.235%
UUC: 35.764%
G (Glycine):
GGA: 29.938%
GGU: 35.802%
GGG: 19.598%
GGC: 14.66%
H (Histidine):
CAU: 66.666%
CAC: 33.333%
I (Isoleucine):
AUA: 24.475%
AUU: 46.503%
AUC: 29.02%
K (Lysine):
AAA: 54.281%
AAG: 45.718%
L (Leucine):
UUA: 17.23%
UUG: 24.754%
CUA: 10.25%
CUU: 27.044%
CUG: 10.468%
CUC: 10.25%
M (Methionine):
AUG: 100.0%
N (Asparagine):
AAU: 57.021%
AAC: 42.978%
P (Proline):
CCA: 36.323%
CCU: 34.792%
CCG: 15.536%
CCC: 13.347%
Q (Glutamine):
CAA: 62.078%
CAG: 37.921%
R (Arginine):
AGA: 29.918%
AGG: 19.877%
CGA: 13.934%
CGU: 17.622%
CGG: 10.45%
CGC: 8.196%
S (Serine):
AGU: 18.542%
AGC: 11.764%
UCA: 25.831%
UCU: 24.296%
UCG: 9.335%
UCC: 10.23%
T (Threonine):
ACA: 34.496%
ACU: 31.976%
ACG: 11.821%
ACC: 21.705%
V (Valine):
GUA: 15.244%
GUU: 44.055%
GUG: 26.153%
GUC: 14.545%
W (Tryptophan):
UGG: 100.0%
Y (Tyrosine):
UAU: 59.87%
UAC: 40.129%
! (Stop):
UAA: 33.333%
UAG: 33.333%
UGA: 33.333%

### Expression Experiments

The protein sequence of the Haematococcus pluvialis beta carotene ketolase (SEQ ID NO:1) was used to obtain its reverse translation considering the specific codon usage of Tagetes as outlined above. In addition, the amino acid sequence of the Pisum sativum Rubisco small subunit (RbcS) transit peptide (SEQ ID NO:2) was reverse translated considering the Tagetes codon usage. All genetic elements were optimized using the software LETO1.0 from Entelechon.

The synthetic DNA fragment (SEQ ID NO:3) corresponding to the optimized RbcS transit peptide coding sequence fused in frame to the optimized Haematococcus pluvialis beta carotene ketolase coding sequence was used in subsequent cloning steps to generate binary plant transformation vector VC-SIW122-13 (SEQ ID NO:4) which is based on the vector VC-LLL544-1qcz backbone (SEQ ID NO:5). All cloning steps were carried out following standard molecular biology protocols.

The T-DNA of VC-SIW122-13 (SEQ ID NO:4) contains a cassette for regeneration of plants under phosphinothricin selection pressure comprising the nos (nopaline synthase) promoter, the coding region of a synthetic phosphinothricin acetyltransferase gene, and the nos terminator. The second expression cassette comprises the Brassica napus plastid-associated protein X (PAPX) promoter, the Pisum sativum RbcS transit peptide coding sequence optimized for expression in Tagetes, the Haematococcus pluvialis beta carotene ketolase gene coding sequence optimized for Tagetes, and the Solanum tuberosum cathepsin D Inhibitor (CAT) terminator.

The following sequences are used:

SEQ ID NO:1: HP BKT (amino acid sequence of Haematococcus pluvialis beta carotene ketolase coding sequence (329 aa))
SEQ ID NO:2: tp-RbcS (Amino acid sequence of Pisum sativum Rubisco small subunit (RbcS) transit peptide)
SEQ ID NO:3: synthetic DNA fragment
Position 1 to 168 Pisum sativum RbcS transit peptide coding sequence optimized for expression in Tagetes (168 bp), underlined
Position 169 to 1158 Haematococcus pluvialis beta carotene ketolase gene, coding sequence optimized for expression in Tagetes (990 bp)
   SEQ ID NO:4: VC-SIW122-13 T-DNA (T-DNA region of binary vector)
Position 1 to 215 Left border
Position 218 to 505 Nos (nopaline synthase) gene promoter
Position 518 to 1069 Phosphinothricin Acetyltransferase synthetic gene/CDS
Position 1139 to 1391 Nos (nopaline synthase) gene terminator
Position 1734 to 2764 Brassica napus plastid-associated protein X (PAPX) promoter
Position 1769 to 2963 Pisum sativum Rubisco small subunit (RbcS) transit peptide coding sequence optimized for expression in Tagetes
Position 2967 to 3956 Haematococcus pluvialis beta-carotene ketolase gene coding sequence optimized for expression in Tagetes
Position 3981 to 4209 Solanum tuberosum Cathepsin D Inhibitor (CAT) terminator
Position 4325 to 4470 Right border
   SEQ ID NO:5: VC-LLL544-1qcz (binary vector backbone)
Position 1 to 146 Right border
Position 320 to 1111 Adenyltransferase [aadA] gene coding region
Position 1560 to 2241 ColE1 E. coli origin of replication
Position 2615 to 2809 pVS1 origin (complementary)
Position 2413 to 5682 pVS1 replicon (complementary)
Position 5691 to 5905 Left border
Position 5606 to 5916 Placeholder (to be replaced by respective T-DNA)

### Example 7

### Analysis of Carotenoids of Tagetes Plants Transformed with Haematococcus pluvialis Beta Carotene Ketolase and of Tagetes Plants Transformed with Haematococcus pluvialis Beta Carotene Ketolase Optimized for Expression in Tagetes erecta

After transformation of Tagetes explants of 31360-2-08 with the binary vector VC-SIW122-13 (comprising Haematococcus pluvialis beta carotene ketolase optimized for expression in Tagetes erecta), several transgenic plants were obtained which were named SIW122. These plants were analyzed for individual yellow carotenoids, the natural occurring and endogenous xanthophylls, and the newly formed ketocarotenoids, especially for astaxanthin, canthaxanthin, echinenone, 3′-hydroxyechinenone, 3-hydroxy-echinenone, phoenicoxanthin (=adonirubin) and adonixanthin.

TABLE 1
Individual carotenoids in petals of transgenic Tagetes SIW122

| | ng Carotenoid/mg | | | | | |
|---|---|---|---|---|---|---|
| | Total | | Phoenico- | beta- | Epoxides | % |
| Plant | Ketos | Astaxanthin | xanthin | Carotene | & Zea | Asta/ketos |
| M1-SIW122-1-4106-2-057 | 1835 | 778 | 544 | 6110 | 5333 | 42% |
| M1-SIW122-1-4106-2-081 | 1805 | 604 | 555 | 4120 | 4484 | 33% |
| M1-SIW122-1-4106-2-087 | 1753 | 843 | 497 | 6400 | 5081 | 48% |
| M1-SIW122-1-4106-2-100 | 2291 | 1056 | 605 | 4901 | 4398 | 46% |
| M1-SIW122-1-4106-2-110 | 2231 | 1075 | 651 | 4838 | 6129 | 48% |
| M1-SIW122-1-4106-2-116 | 2617 | 1554 | 615 | 10255 | 5349 | 59% |

Listed values represent individual carotenoids in percent of total carotenoids, extracted and analyzed as described. The carotenoid extract was prepared from fully opened Tagetes flowers. Values refer to dry weight.

TABLE 2
Individual ketocarotenoids in petals of transgenic Tagetes SIW122

| | ng Carotenoid/mg | | | | | |
|---|---|---|---|---|---|---|
| | | Adoni- | Cantha- | | Phoenico- | Total |
| Plant | Astaxanthin | xanthin | xanthin | 3′ Hydroxy | xanthin | ketos |
| M1-SIW122-1-4106-2-057 | 778 | 62 | 409 | 42 | 544 | 1835 |
| M1-SIW122-1-4106-2-081 | 604 | 40 | 560 | 46 | 555 | 1805 |
| M1-SIW122-1-4106-2-087 | 843 | 64 | 307 | 41 | 497 | 1753 |
| M1-SIW122-1-4106-2-100 | 1056 | 64 | 523 | 43 | 605 | 2291 |
| M1-SIW122-1-4106-2-110 | 1075 | 54 | 409 | 42 | 651 | 2231 |
| M1-SIW122-1-4106-2-116 | 1554 | 80 | 342 | 25 | 615 | 2617 |

TABLE 3
Average ketocarotenoids in petals of transgenic Tagetes SIW122 compared to average
ketocarotenoids in petals of transgenic Tagetes MS259. Values refer to dry weight.
Transgenic plants MS259 carry the wild type, not codon-optimized Haematococcus
Ketolase gene as described in WO2004018693.

| | | | | | | |
|---|---|---|---|---|---|---|
| | | | 3′Hydorxy- | 3′Hydroxy- | Phoenico- | Total |
| Astaxanthin | Adonixanthin | Canthaxanthin | echinenon | echinenon | xanthin | Ketos |

ng Carotenoid/mg

| | | | | | | | |
|---|---|---|---|---|---|---|---|
| SIW122 | 985 | 61 | 425 | 40 | 0 | 578 | 2089 |
| MS259 | 373 | 39 | 251 | 53 | 0 | 328 | 1045 |

Percentages

| | | | | | | | |
|---|---|---|---|---|---|---|---|
| SIW122 | 7% | 0% | 3% | 0% | 0% | 4% | 15.3% |
| MS259 | 2% | 0% | 2% | 0% | 0% | 2% | 6.9% |

TABLE 4
Average carotenoids in petals of transgenic Tagetes SIW122 compared to average carotenoids
in petals of transgenic Tagetes MS259. Values refer to dry weight.

| | | | | | | | | | | | |
|---|---|---|---|---|---|---|---|---|---|---|---|
| | | | | b- | | | | | Total | Total | |
| | Zea- | Violaxanthin + | Anthera- | Crypto- | beta- | gamma- | Lyco- | | caros no | caros with | Epoxides |
| Lutein | xanthin | Neoxanthin | xanthin | xanthin | Carotene | Carotene | pene | Phytoene | phytoene | phytoene | & Zea |

ng Carotenoid/mg

| | | | | | | | | | | | | |
|---|---|---|---|---|---|---|---|---|---|---|---|---|
| SIW122 | 0 | 1759 | 2437 | 933 | 37 | 6104 | 0 | 329 | 1136 | 13688 | 14824 | 5129 |
| MS259 | 0 | 1853 | 1933 | 1448 | 56 | 8204 | 223 | 316 | 1372 | 15077 | 16449 | 5234 |

Percentages

| | | | | | | | | | | | | |
|---|---|---|---|---|---|---|---|---|---|---|---|---|
| SIW122 | 0 | 13 | 18 | 7 | 0 | 45 | 0 | 2 | 8 | 100 | 108 | 37 |
| MS259 | 0 | 12 | 13 | 10 | 0 | 54 | 1 | 2 | 9 | 100 | 109 | 35 |

Legend for table 1 to table 4:
“Total caros”: total amounts of all carotenoids extracted from Tagetes petals
“Total ketos”: sum of all ketocarotenoids extracted from Tagetes petals (canthaxanthin, phoenicoxanthin, astaxanthin, adonixanthin, echinenone, 3′- and 3-hydroxy-echinenone).
“Zea”: designation for zeaxanthin
“3′-Hydroxy”: designation for 3′-hydroxyechinenone
“Epoxides”: designation for the combined concentration of the carotenoid epoxides violaxanthin, antheraxanthin and neoxanthin

### Example 8

### Expression of the Ketolase Gene from Scenedesmus vacuolatus Optimized for Expression in Tagetes erecta

The protein sequence of the Scenedesmus vacuolatus beta carotene ketolase (SEQ ID NO:6) was used to obtain its reverse translation considering the specific codon usage of Tagetes as outlined in Example 4. In addition, the amino acid sequence of the Pisum sativum Rubisco small subunit (RbcS) transit peptide (SEQ ID NO:2) was reverse translated considering the Tagetes codon usage.

The synthetic DNA fragment (SEQ ID NO:7) corresponding to the optimized RbcS transit peptide coding sequence fused in frame to the optimized Scenedesmus vacuolatus beta carotene ketolase coding sequence was used in subsequent cloning steps to generate binary plant transformation vector VC-SIW182-6 (SEQ ID NO:8) which is based on the vector VC-LLL544-1qcz backbone (SEQ ID NO:5). All cloning steps were carried out following standard molecular biology protocols.

The T-DNA of VC-SIW182-6 (SEQ ID NO:8) contains a cassette for regeneration of plants under phosphinothricin selection pressure comprising the nos (nopaline synthase) promoter, the coding region of a synthetic phosphinothricin acetyltransferase gene, and the nos terminator. The second expression cassette comprises the Brassica napus plastid-associated protein X (PAPX) promoter, the Pisum sativum RbcS transit peptide coding sequence optimized for expression in Tagetes, the Scenedesmus vacuolatus beta carotene ketolase gene coding sequence optimized for Tagetes, and the Solanum tuberosum cathepsin D Inhibitor (CAT) terminator.

The following sequences are used: SEQ ID NO:2 and 5 from Example 4.

SEQ ID NO:6: SV211 BKT (amino acid sequence of Scenedesmus vacuolatus beta carotene ketolase coding sequence (331 aa)
SEQ ID NO:7: synthetic DNA fragment
Position 1 to 171 Pisum sativum RbcS transit peptide coding sequence optimized for expression in Tagetes (171 bp), underlined
Position 172 to 1158 Scenedesmus vacuolatus beta carotene ketolase gene, coding sequence optimized for expression in Tagetes (996 bp)
   SEQ ID NO:8: VC-SIW182-6 (T-DNA region of binary vector)
Position 1 to 215 Left border
Position 218 to 505 Nos (nopaline synthase) gene promoter
Position 518 to 1069 Phosphinothricin Acetyltransferase synthetic gene/CDS
Position 1139 to 1391 Nos (nopaline synthase) gene terminator
Position 1582 to 1813 Solanum tuberosum Cathepsin D Inhibitor (CAT) terminator (complementary)
Position 1835 to 2830 Scenedesmus vacuolatus beta-carotene ketolase gene coding sequence optimized for expression in Tagetes (complementary)
Position 2831 to 3001 Pisum sativum Rubisco small subunit (RbcS) transit peptide coding sequence optimized for expression in Tagetes (complementary)
Position 3033 to 4063 Brassica napus plastid-associated protein X (PAPX) promoter (complementary)
Position 4331 to 4476 Right border

### Example 9

### Analysis of Carotenoids of Tagetes Plants Transformed with Scenedesmus vacuolatus Beta Carotene Ketolase and of Tagetes Plants Transformed with Scenedesmus vacuolatus Beta Carotene Ketolase Optimized for Expression in Tagetes erecta

After transformation of Tagetes explants of 31360-2-08 with the binary vector VC-SIW182-6 (comprising Scenedesmus vacuolatus beta carotene ketolase optimized for expression in Tagetes erecta), several transgenic plants were obtained which were named SIW182. These plants were analyzed for individual yellow carotenoids, the natural occurring and endogenous xanthophylls, and the newly formed ketocarotenoids, especially for astaxanthin, canthaxanthin, echinenone, 3′-hydroxechinenone, 3-hydroxy-echinenone, phoenicoxanthin (=adonirubin) and adonixanthin.

TABLE 5
Individual carotenoids in petals of transgenic Tagetes SIW182

| | ng Carotenoid/mg | | | | | | |
|---|---|---|---|---|---|---|---|
| | Total | | Phoenico- | beta- | | Zea- | % |
| Plant | Ketos | Astaxanthin | xanthin | Carotene | Epoxides | xanthin | Asta/ketos |
| M1-SIW182-6-3 | 7823 | 5530 | 1570 | 841 | 1099 | 96 | 75 |
| M1-SIW182-6-4 | 8076 | 5254 | 1968 | 602 | 812 | 112 | 79 |
| M1-SIW182-6-6 | 7778 | 4340 | 2370 | 462 | 385 | 130 | 84 |
| M1-SIW182-6-7 | 6755 | 4578 | 1425 | 805 | 877 | 99 | 76 |
| M1-SIW182-6-8 | 8696 | 5925 | 1933 | 577 | 962 | 118 | 80 |
| M1-SIW182-6-10 | 7985 | 5721 | 1742 | 157 | 564 | 67 | 87 |
| M1-SIW182-6-18 | 11518 | 8404 | 2350 | 318 | 1198 | 86 | 85 |
| M1-SIW182-6-27 | 8815 | 6650 | 1615 | 321 | 1201 | 80 | 82 |
| M1-SIW182-6-32 | 6908 | 4870 | 1442 | 302 | 753 | 76 | 81 |
| M1-SIW182-6-39 | 9089 | 7537 | 1238 | 143 | 1337 | 65 | 83 |
| M1-SIW182-6-45 | 8237 | 6930 | 1008 | 236 | 1711 | 85 | 78 |

Listed values represent individual carotenoids in percent of total carotenoids, extracted and analyzed as described. The carotenoid extract was prepared from fully opened Tagetes flowers. Values refer to dry weight.

TABLE 6
Individual ketocarotenoids in petals of transgenic Tagetes SIW182

| | ng Carotenoid/mg | | | |
|---|---|---|---|---|
| | | | | Total |
| Plant | Astaxanthin | Canthaxanthin | Phoenicoxanthin | ketos |
| M1-SIW182- | 5530 | 723 | 1570 | 7823 |
| 6-3 | | | | |
| M1-SIW182- | 5254 | 854 | 1968 | 8076 |
| 6-4 | | | | |
| M1-SIW182- | 4340 | 1068 | 2370 | 7778 |
| 6-6 | | | | |
| M1-SIW182- | 4578 | 752 | 1425 | 6755 |
| 6-7 | | | | |
| M1-SIW182- | 5925 | 838 | 1933 | 8696 |
| 6-8 | | | | |
| M1-SIW182- | 5721 | 522 | 1742 | 7985 |
| 6-10 | | | | |
| M1-SIW182- | 8404 | 764 | 2350 | 11518 |
| 6-18 | | | | |
| M1-SIW182- | 6650 | 550 | 1615 | 8815 |
| 6-27 | | | | |
| M1-SIW182- | 4870 | 596 | 1442 | 6908 |
| 6-32 | | | | |
| M1-SIW182- | 7537 | 314 | 1238 | 9089 |
| 6-39 | | | | |
| M1-SIW182- | 6930 | 299 | 1008 | 8237 |
| 6-45 | | | | |

### Example 10

### Expression of the Ketolase Gene from Chlorella zoofingiensis Optimized for Expression in Tagetes erecta

The protein sequence of the Chlorella zoofingiensis beta carotene ketolase (SEQ ID NO:9) was used to obtain its reverse translation considering the specific codon usage of Tagetes as outlined in Example 4. In addition, the amino acid sequence of the Pisum sativum Rubisco small subunit (RbcS) transit peptide (SEQ ID NO:2) was reverse translated considering the Tagetes codon usage.

The synthetic DNA fragment (SEQ ID NO:10) corresponding to the optimized RbcS transit peptide coding sequence fused in frame to the optimized Chlorella zoofingiensis beta carotene ketolase coding sequence was used in subsequent cloning steps to generate binary plant transformation vector VC-SIW198-1 (SEQ ID NO:11) which is based on the vector VC-LLL544-1qcz backbone (SEQ ID NO:5). All cloning steps were carried out following standard molecular biology protocols.

The T-DNA of VC-SIW198-1 (SEQ ID NO:11) contains a cassette for regeneration of plants under phosphinothricin selection pressure comprising the nos (nopaline synthase) promoter, the coding region of a synthetic phosphinothricin acetyltransferase gene, and the nos terminator. The second expression cassette comprises the Brassica napus plastid-associated protein X (PAPX) promoter, the Pisum sativum RbcS transit peptide coding sequence optimized for expression in Tagetes, the Chlorella zoofingiensis beta carotene ketolase gene coding sequence optimized for Tagetes, and the Solanum tuberosum cathepsin D Inhibitor (CAT) terminator.

The following sequences were used in addition to SEQ ID NO:2 and 5 of Example 4

SEQ ID NO:9: CZ BKT (amino acid sequence of Chlorella zoofingiensis beta carotene ketolase coding sequence (312 aa)
SEQ ID NO:10: synthetic DNA fragment
Position 1 to 171 Pisum sativum RbcS transit peptide coding sequence optimized for expression in Tagetes (171 bp), underlined
Position 172 to 1110 Chlorella zoofingiensis beta carotene ketolase gene, coding sequence optimized for expression in Tagetes (939 bp)
   SEQ ID NO:11: VC-SIW198-1 (T-DNA region of binary vector)
Position 1 to 215 Left border
Position 218 to 505 Nos (nopaline synthase) gene promoter
Position 518 to 1069 Phosphinothricin Acetyltransferase synthetic gene/CDS
Position 1139 to 1391 Nos (nopaline synthase) gene terminator
Position 1582 to 1813 Solanum tuberosum Cathepsin D Inhibitor (CAT) terminator (complementary)
Position 1823 to 2761 Chlorella zoofingiensis beta-carotene ketolase gene coding sequence optimized for expression in Tagetes (complementary)
Position 2762 to 2932 Pisum sativum Rubisco small subunit (RbcS) transit peptide coding sequence optimized for expression in Tagetes (complementary)
Position 2968 to 3998 Brassica napus plastid-associated protein X (PAPX) promoter (complementary)
Position 4266 to 4411 Right border

### Example 11

### Analysis of Carotenoids of Tagetes Plants Transformed with Chlorella zoofingiensis Beta Carotene Ketolase and of Tagetes Plants Transformed with Chlorella zoofingiensis Beta Carotene Ketolase Optimized for Expression in Tagetes erecta

After transformation of Tagetes explants of 31360-2-08 with the binary vector VC-SIW198-1 (comprising Chlorella zoofingiensis beta carotene ketolase optimized for expression in Tagetes erecta), several transgenic plants were obtained which were named SIW198. These plants were analyzed for individual yellow carotenoids, the natural occurring and endogenous xanthophylls, and the newly formed ketocarotenoids, especially for astaxanthin, canthaxanthin, echinenone, 3′-hydroxechinenone, 3-hydroxy-echinenone, phoenicoxanthin (=adonirubin) and adonixanthin.

Tagetes plants SIW122 exhibit a clearly orange flower phenotype due to the accumulation of ketocarotenoids produced in the petals of the flowers. This phenotype is even more pronounced in Tagetes plants SIW182. Several flowers of SIW182 are clearly intensely red due to the high concentrations of ketocarotenoids in those petals. In contrast, flowers of SIW 198 showed only minor to moderate phenotype. Many flowers showed a yellow/orangish phenotype which is not much distinguishable from the control plants. A few flowers showed a slight orange phenotype due to low amounts of ketocarotenoids accumulating in the flowers.

### Example 12

### Expression of the Ketolase Gene from Chlamydomonas reinhardtii Optimized for Expression in Tagetes erecta

The protein sequence of the Chlamydomonas reinhardtii beta carotene ketolase (SEQ ID NO:12) was used to obtain its reverse translation considering the specific codon usage of Tagetes as outlined in Example 4. In addition, the amino acid sequence of the Pisum sativum Rubisco small subunit (RbcS) transit peptide (SEQ ID NO:2) was reverse translated considering the Tagetes codon usage.

The synthetic DNA fragment (SEQ ID NO:13) corresponding to the optimized RbcS transit peptide coding sequence fused in frame to the optimized Chlamydomonas reinhardtii beta carotene ketolase coding sequence was used in subsequent cloning steps to generate binary plant transformation vector VC-SIW195-1 (SEQ ID NO:14) which is based on the vector VC-LLL544-1qcz backbone (SEQ ID NO:5). All cloning steps were carried out following standard molecular biology protocols.

The T-DNA of VC-SIW195-1 (SEQ ID NO:14) contains a cassette for regeneration of plants under phosphinothricin selection pressure comprising the nos (nopaline synthase) promoter, the coding region of a synthetic phosphinothricin acetyltransferase gene, and the nos terminator. The second expression cassette comprises the Brassica napus plastid-associated protein X (PAPX) promoter, the Pisum sativum RbcS transit peptide coding sequence optimized for expression in Tagetes, the Chlamydomonas reinhardtii beta carotene ketolase gene coding sequence optimized for Tagetes, and the Solanum tuberosum cathepsin D Inhibitor (CAT) terminator.

The following sequences were used in addition to SEQ ID NO:2 and 5 of Example 4

SEQ ID NO:12: CR BKT (amino acid sequence of Chlamydomonas reinhardtii beta carotene ketolase coding sequence (328 aa)

The ketolase version used has been shortened at the C-terminus of the protein. When aligned with known ketolases of public databases, the Chlamydomonas ketolase showed an extended C-terminus not found in other ketolases of proven function. In addition, the extension showed longer stretches of repetitive alanine, serine and glycine stretches.

SEQ ID NO:13: synthetic DNA fragment
Position 1 to 171 Pisum sativum RbcS transit peptide coding sequence optimized for expression in Tagetes (171 bp), underlined
Position 172 to 1158 Chlamydomonas reinhardtii beta carotene ketolase gene, 3′-shortened coding sequence optimized for expression in Tagetes (987 bp)
   SEQ ID NO:14: VC-SIW195-1 (T-DNA region of binary vector)
Position 1 to 215 Left border
Position 218 to 505 Nos (nopaline synthase) gene promoter
Position 518 to 1069 Phosphinothricin Acetyltransferase synthetic gene/CDS
Position 1139 to 1391 Nos (nopaline synthase) gene terminator
Position 1582 to 1813 Solanum tuberosum Cathepsin D Inhibitor (CAT) terminator (complementary)
Position 1835 to 2821 Chlamydomonas reinhardtii beta-carotene ketolase gene coding sequence optimized for expression in Tagetes (complementary)
Position 2822 to 2992 Pisum sativum Rubisco small subunit (RbcS) transit peptide coding sequence optimized for expression in Tagetes (complementary)
Position 3028 to 4058 Brassica napus plastid-associated protein X (PAPX) promoter (complementary)
Position 4326 to 4471 Right border

### Example 13

### Analysis of Carotenoids of Tagetes Plants Transformed with Chlamydomonas reinhardtii Beta Carotene Ketolase and of Tagetes Plants Transformed with Chlamydomonas reinhardtii Beta Carotene Ketolase Optimized for Expression in Tagetes erecta

After transformation of Tagetes explants of 31360-2-09 with the binary vector VC-SIW195-1 (comprising Chlamydomonas reinhardtii beta carotene ketolase optimized for expression in Tagetes erecta), several transgenic plants were obtained which were named SIW195. These plants were analyzed for individual yellow carotenoids, the natural occurring and endogenous xanthophylls, and the newly formed ketocarotenoids, especially for astaxanthin, canthaxanthin, echinenone, 3′-hydroxechinenone, 3-hydroxy-echinenone, phoenicoxanthin (=adonirubin) and adonixanthin.

### Example 14

### Vector Construction for Coexpression Experiments of a Beta-Cyclase and a Ketolase Gene

Vectors used for expression of full-length tomato B-gene lycopene beta-cyclase and full length Scenedesmus ketolase gene in plants (overexpression) are designed to overexpress the B-gene lycopene beta-cyclase and the Scenedesmus ketolase under control of suitable promoters and are of two general types, biolistic and binary, depending on the plant transformation method to be used.

For biolistic transformation (biolistic vectors), the requirements are as follows:

1. a backbone with a bacterial selectable marker (typically, an antibiotic resistance gene) and origin of replication functional in Escherichia coli (E. coli; e.g., ColE1), and
2. a plant-specific portion consisting of:
a. a gene expression cassette consisting of a promoter (e.g., ZmUBlint MOD), the gene of interest (typically, a full-length cDNA) and a transcriptional terminator (e.g., Agrobacterium tumefaciens nos terminator);
b. a plant selectable marker cassette, consisting of a suitable promoter, selectable marker gene (e.g., pat; D-amino acid oxidase; dao1) and transcriptional terminator (eg. nos terminator).

Vectors designed for transformation by Agrobacterium tumefaciens (A. tumefaciens; binary vectors) consist of:

1. a backbone with a bacterial selectable marker functional in both E. coli and A. tumefaciens (e.g., spectinomycin resistance mediated by the aadA gene) and two origins of replication, functional in each of aforementioned bacterial hosts, plus the A. tumefaciens virG gene;
2. a plant-specific portion as described for biolistic vectors above, except in this instance this portion is flanked by A. tumefaciens right and left border sequences which mediate transfer of the DNA flanked by these two sequences to the plant.

### Base Vector Used for Cloning of Overexpression Constructs

SEQ ID NO. 24: VC-LLL544-1qcz (binary vector backbone)
Position 1 to 146 Right border
Position 320 to 1111 Adenyltransferase [aadA] gene coding region
Position 1560 to 2241 ColE1 E. coli origin of replication
Position 2615 to 2809 pVS1 origin (complementary)
Position 2413 to 5682 pVS1 replicon (complementary)
Position 5691 to 5905 Left border
Position 5606 to 5916 Placeholder (to be replaced by respective T-DNA)
   SEQ ID NO. 25: T-DNA of binary vector with expression cassette
Position 1 to 146 Right border
Position 253 to 554 LB3 gene terminator, complementary
Position 583-1578 Scenedesmus vacuolatus 211-8b ketolase optimized CDS, complementary
Position 1579-1774 Pea rubisco small sub-unit transit peptide CDS, complementary
Position 1781-2811 Brassica napus PAPX promoter, complementary
Position 3015-4466 Antirrhinum majus fiddlehead promoter
Position 4535-6028 tomato B-gene lycopene beta-cyclase
Position 6094-6395 LB3 gene terminator
Position 6488-6740 NOS terminator, complementary
Position 6810-7361 phosphinothricin acetyltransferase CDS, complementary
Position 7374-7661 NOS promoter, complementary
Position 7664-7878 Left border

### Example 15

### HPLC Analysis of Free Carotenoids

The analysis of samples prepared according to the procedure described above was done under the following conditions:

### HPLC Conditions:

HPLC column: Prontosil C30, 250×4.6 mm, (Bischoff, Leonberg, Germany)
Flow rate: 1.0 ml/min
Eluents: Solvent A—100% methanol Solvent B—80% methanol, 0.2% ammoniumacetate Solvent C—100% t-butyl-methylether
Detection: 300-530 nm

### Gradienten Profile:

| Time (min) | Flow rate | % Solvent A | % Solvent B | % Solvent C |
|---|---|---|---|---|
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids are:

violaxanthin at about 11.7 min, astaxanthin at about 17.7 min, adonixanthin at about 19 min, adonirubin at about 19.9 min, zeaxanthin at about 21 min.

After transformation of Tagetes explants of 31360-2-09-08ApxM with the binary vector VC-SUL80, several transgenic plants were obtained which were named M4-SUL80-, 15, 19, 21, 29, 37, 41, 43, 48, 53, 57, 69, 73, 74, 76, 84, 85 & 88. These plants were analyzed for individual yellow carotenoids, the natural occurring and endogenous xanthophylls, and the newly formed ketocarotenoids, especially for astaxanthin, canthaxanthin, echinenone, 3′-hydroxechinenone, 3-hydroxechinenone, phoenicoxanthin (=adonirubin) and adonixanthin.

Listed values represent individual carotenoids in percent of total carotenoids, extracted and analyzed as described. The carotenoid extract was prepared from fully opened Tagets flowers. Value refer to fresh weight.

### Legend for Table7 and Following:

“total caros”: total amounts of all carotenoids extracted from Tagets petals
“Ketos”: sum of all ketocarotenoids extracted from Tagetes petals (canthaxanthin, phoenicoxanthin, Aastaxanthin, adonixanthin, echinenone, 3′- and 3-hydroxyechinenone).
“A” and “Asta”: designation for the ketocarotenoid astaxanthin
“Adoni”: designation for the ketocarotenoid adonixanthin
“P” and “Phoenico”: designation for the ketocarotenoid phoenicoxanthin, also known as adonirubin
“C” and “Cantha”: designation for the ketocarotenoid canthaxanthin
“bC”: designation for beta-carotene
“Cryp”: designation for beta-cryptoxanthin
“Zea”: designation for zeaxanthin
“Cantha” designation for canthaxanthin
“3′-Hydroxy” and “HO-echi”: designation for 3′-hydroxyechinenone
“b-Crypto” bezeichnet beta-Cryptoxanthin
“Epoxides”: designation for the combined concentration of the carotenoid epoxides violaxanthin, antheraxanthin and neoxanthin
“DW” stands for dry weight

TABLE 7
Individual carotenoids in petals of transgenic Tagetes UL80 and appropriate wild types

| | ng/mg | | | | | | | | |
|---|---|---|---|---|---|---|---|---|---|
| | DW | % | % | % | % | % | % | % | % |
| Plant | total caros | Ketos | Asta | A + P | A + P + C | bC | Cryp | Zea | Epoxides |
| M4-SUL80-015 | 15832 | 91% | 68% | 85% | 91% | 3% | 0% | 1% | 3% |
| M4-SUL80-019 | 12115 | 79% | 61% | 74% | 79% | 8% | 0% | 2% | 7% |
| M4-SUL80-021 | 12734 | 88% | 64% | 82% | 88% | 5% | 0% | 1% | 3% |
| M4-SUL80-029 | 14145 | 83% | 62% | 78% | 83% | 6% | 0% | 2% | 6% |
| M4-SUL80-037 | 18976 | 89% | 67% | 84% | 89% | 3% | 0% | 1% | 2% |
| M4-SUL80-041 | 15487 | 89% | 65% | 82% | 89% | 5% | 0% | 1% | 3% |
| M4-SUL80-043 | 12465 | 80% | 60% | 75% | 80% | 5% | 0% | 1% | 8% |
| M4-SUL80-048 | 11631 | 82% | 61% | 77% | 82% | 5% | 0% | 1% | 6% |
| M4-SUL80-053 | 15119 | 79% | 61% | 74% | 79% | 9% | 0% | 2% | 7% |
| M4-SUL80-057 | 15241 | 83% | 63% | 79% | 83% | 5% | 0% | 2% | 7% |
| M4-SUL80-069 | 16655 | 83% | 63% | 78% | 83% | 6% | 0% | 2% | 6% |
| M4-SUL80-073 | 16166 | 83% | 66% | 79% | 83% | 5% | 0% | 2% | 7% |
| M4-SUL80-074 | 16313 | 80% | 64% | 76% | 80% | 5% | 0% | 2% | 9% |
| M4-SUL80-076 | 20743 | 87% | 66% | 82% | 87% | 2% | 0% | 3% | 7% |
| M4-SUL80-084 | 17107 | 85% | 68% | 81% | 85% | 3% | 0% | 1% | 7% |
| M4-SUL80-085 | 13267 | 75% | 58% | 72% | 75% | 5% | 0% | 4% | 12% |
| M4-SUL80-088 | 15141 | 78% | 61% | 74% | 78% | 5% | 0% | 3% | 11% |
| WT-M4-control #1 | 9179 | 0% | 0% | 0% | 0% | 11% | 0% | 44% | 44% |
| WT-M4-control #2 | 8922 | 0% | 0% | 0% | 0% | 11% | 0% | 45% | 42% |
| WT-M4-control #3 | 9462 | 0% | 0% | 0% | 0% | 8% | 0% | 43% | 47% |
| WT-M4-control #4 | 9475 | 0% | 0% | 0% | 0% | 7% | 0% | 42% | 49% |

Listed values represent individual carotenoids in percent of total carotenoids, extracted and analyzed as described. The carotenoid extract was prepared from fully opened Tagetes flowers. Value refer to dry weight.

TABLE 8
Individual ketocarotenoids in petals of transgenic Tagetes UL80

| | | | | | HO- | |
|---|---|---|---|---|---|---|
| Plant | Asta | Adoni | Phoenico | Cantha | echi | Ketos |
| M4-SUL80-015 | 68% | 0% | 18% | 6% | 0% | 91% |
| M4-SUL80-019 | 61% | 0% | 13% | 5% | 0% | 79% |
| M4-SUL80-021 | 64% | 0% | 18% | 5% | 0% | 88% |
| M4-SUL80-029 | 62% | 0% | 15% | 5% | 0% | 83% |
| M4-SUL80-037 | 67% | 0% | 17% | 5% | 0% | 89% |
| M4-SUL80-041 | 65% | 0% | 17% | 6% | 0% | 89% |
| M4-SUL80-043 | 60% | 0% | 16% | 5% | 0% | 80% |
| M4-SUL80-048 | 61% | 0% | 16% | 5% | 0% | 82% |
| M4-SUL80-053 | 61% | 0% | 14% | 5% | 0% | 79% |
| M4-SUL80-057 | 63% | 0% | 15% | 5% | 0% | 83% |
| M4-SUL80-069 | 63% | 0% | 15% | 5% | 0% | 83% |
| M4-SUL80-073 | 66% | 0% | 13% | 4% | 0% | 83% |
| M4-SUL80-074 | 64% | 0% | 13% | 4% | 0% | 80% |
| M4-SUL80-076 | 66% | 0% | 16% | 5% | 0% | 87% |
| M4-SUL80-084 | 68% | 0% | 14% | 4% | 0% | 85% |
| M4-SUL80-085 | 58% | 0% | 13% | 3% | 0% | 75% |
| M4-SUL80-088 | 61% | 0% | 13% | 4% | 0% | 78% |

Listed values represent individual carotenoids in percent of total carotenoids, extracted and analyzed as described. The carotenoid extract was prepared from fully opened Tagetes flowers. Value refer to dry weight.

### Example 16

### Isolation of the Primary Sequence of the AFI Promoter from Antirrhinum majus Driving Epidermis-Specific Expression in Floral Organ

The DNA fragment corresponding to the AFI promoter (Efremova et al (2004) Plant Mol Biol 56: 821-837) (SEQ ID NO. 28) was amplified on genomic Antirrhinum majus DNA via PCR using the specific primers AFIfor (SEQ ID NO. 26) and AFIrev (SEQ ID NO. 27). To both primers restriction sites for cloning were added. PCR was carried out using a standard protocol. PCR-amplification resulted in a 1452 bp-promoter fragment (SEQ ID NO. 28), which was used in subsequent cloning steps to generate the binary plant transformation vector VC-SBT477, which is based on the vector VC-LLL544-1qcz backbone (SEQ ID NO. 5). All cloning steps were carried out following standard molecular biology protocols. Since VC-LLL544-1qcz is a modified Gateway destination vector (Invitrogen) the final assembly of VC VC-SBT477 was carried out by site directed recombination according to the manufacturer's protocol (Invitrogen). The T-DNA of VC-SBT477 (SEQ ID NO. 29) contains a cassette for regeneration of plants under phosphinothricin selection pressure comprising the nos promoter, the coding region of a synthetic phosphinothricin acetyltransferase gene, and the octopine synthase terminator of transcription. The second expression cassette comprises the AFI promoter, the plastid transit peptide from the small subunit of pea rubisco, the Scenedesmus vacuolatus (SAG211-8b) beta-carotene ketolase coding region (SEQ ID NO. 30) and the CAT (potato cathepsin D inhibitor gene) terminator of transcription.

| SEQ ID NO. 26 | |
|---|---|
| AFIfor | CTGGTACCACTTTCGTAATCATATTACCCAACCG |
| SEQ ID NO. 27 | |
| AFIrev | CTGGATCCGTTGTTTGGTTTGAGGATTGAGATGA |

SEQ ID NO. 28 Antirrhinum majus AFI promoter fragment (1452 bp)
SEQ ID NO. 29 T-DNA region of binary vector VC-SBT477 (4903 bp)
Position 1 to 215 Left border
Position 218 to 505 Nos (nopaline synthase) gene promoter
Position 518 to 1069 Phosphinothricin Acetyltransferase synthetic gene/CDS
Position 1139 to 1391 Nos (nopaline synthase) gene terminator
Position 1745 to 3196 AFI promoter
Position 3234 to 3401 Pea rbcS (coding for RuBisCO small subunit) transit peptide
Position 3405 to 4400 Scenedesmus vacuolatus (SAG211-8b) beta-carotene ketolase
Position 4421 to 4652 CAT (potato cathepsin D inhibitor gene) terminator
Position 4758 to 4903 Right border
   SEQ ID NO. 30 Scenedesmus vacuolatus (SAG211-8b) beta-carotene ketolase

### Example 17

### Biochemical Analytics of Tagetes Explants of 31360-2-09-8-8ApxM Transformed with the Binary Vector VC-SBT477

Analysis of Xanthophyll and Ketocarotinoid was performed as described above in example 3. After transformation of Tagetes explants of 31360-2-09-8-8ApxM with the binary vector VC-SBT477, several transgenic plants were obtained which were named M4-BT477-2, 11, 14, 26, 27 & 29. These plants were analyzed for individual yellow carotenoids, the natural occurring and endogenous xanthophylls, and the newly formed ketocarotenoids, especially for astaxanthin, canthaxanthin, echinenone, 3′-hydroxechinenone, 3-hydroxechinenone, phoenicoxanthin (=adonirubin) and adonixanthin (Table 9 and Table 10).

### Legend for Table 9 and 10:

“total caros”: total amounts of all carotenoids extracted from Tagets petals
“Ketos”: sum of all ketocarotenoids extracted from Tagetes petals (canthaxanthin, phoenicoxanthin, astaxanthin, adonixanthin, echinenone, 3′- and 3-hydroxyechinenone).
“A” and “Asta”: designation for the ketocarotenoid astaxanthin
“Adoni”: designation for the ketocarotenoid adonixanthin
“P” and “Phoenico”: designation for the ketocarotenoid phoenicoxanthin, also known as adonirubin
“C” and “Cantha”: designation for the ketocarotenoid canthaxanthin
“bC”: designation for beta-carotene
“Cryp”: designation for beta-cryptoxanthin
“Zea”: designation for zeaxanthin
“Cantha”: designation for canthaxanthin
“Lyc”: designation for lycopin
“3′-Hydroxy” and “HO-echi”: designation for 3′-hydroxyechinenone
“b-Crypto”: designation for beta-Cryptoxanthin “Epoxides”: designation for the combined concentration of the carotenoid epoxides violaxanthin, antheraxanthin and neoxanthin
“DW” stands for dry weight

TABLE 9
Individual carotenoids in petals of transgenic Tagetes BT477

| | ng/mg | | | | | | | | |
|---|---|---|---|---|---|---|---|---|---|
| | DW | % | % | % | % | % | % | % | % |
| Plant | total caros | Ketos | Asta | A + P | A + P + C | bC | Cryp | Zea | Epoxides |
| M4-BT477-2 | 11381 | 57% | 44% | 53% | 57% | 4% | 0% | 2% | 34% |
| M4-BT477-11 | 13298 | 65% | 53% | 61% | 65% | 5% | 0% | 4% | 25% |
| M4-BT477-14 | 12123 | 48% | 36% | 44% | 48% | 6% | 0% | 6% | 39% |
| M4-BT477-26 | 8255 | 57% | 44% | 53% | 57% | 6% | 0% | 4% | 33% |
| M4-BT477-27 | 12173 | 60% | 44% | 55% | 60% | 5% | 0% | 3% | 32% |
| M4-BT477-29 | 11761 | 58% | 45% | 54% | 58% | 5% | 0% | 5% | 32% |

Listed values represent individual carotenoids in percent of total carotenoids, extracted and analyzed as described. The carotenoid extract was prepared from fully opened Tagetes flowers. Values refer to dry weight.

TABLE 10
Individual ketocarotenoids in petals of transgenic Tagetes BT477

| | | | | | HO- | |
|---|---|---|---|---|---|---|
| Plant | Asta | Adoni | Phoenico | Cantha | echi | Ketos |
| M4-BT477-2 | 44% | 0% | 10% | 3% | 0% | 57% |
| M4-BT477-11 | 53% | 0% | 8% | 4% | 0% | 65% |
| M4-BT477-14 | 36% | 0% | 9% | 4% | 0% | 48% |
| M4-BT477-26 | 44% | 0% | 9% | 4% | 0% | 57% |
| M4-BT477-27 | 44% | 0% | 10% | 5% | 0% | 60% |

Listed values represent individual carotenoids in percent of total carotenoids, extracted and analyzed as described. The carotenoid extract was prepared from fully opened Tagetes flowers. Values refer to dry weight.

Annex 1

| Molecule | | | |
|---|---|---|---|
| type | Sequence name in database | Original DB Accession number | Description in database |
| dna | 41_BKT_O23973 | GENESEQ_DNA\|AAV34437 | H. pluvialis beta-carotene C-4-oxygenase |
| | | | enzyme (crtO) encoding cDNA. |
| protein | 41_BKT_O23973 | SPTREMBL\|O23973_HAEPL | Beta-carotene C-4 oxygenase (Ketolase). |
| protein | GENESEQ_PROT\|AAB62157 | GENESEQ_PROT\|AAB62157 | Transit peptide and beta-carotene C-4- |
| | | | oxygenase fusion protein. |
| dna | GENESEQ_PROT\|AAO16024 | GENESEQ_DNA\|ABT14221 | Brevundimonas aurantiaca Beta-carotene C4 |
| | | | oxygenase gene. |
| protein | GENESEQ_PROT\|AAO16024 | GENESEQ_PROT\|AAO16024 | Brevundimonas aurantiaca Beta-carotene C4 |
| | | | oxygenase. |
| protein | GENESEQ_PROT\|AAR79058 | GENESEQ_PROT\|AAR79058 | 3 hydroxy-beta-ionone ring methylene to keto |
| | | | group converting peptide. |
| dna | GENESEQ_PROT\|AAR92098 | unknown id | Beta-ionone 4-methylene gp. to keto gp. |
| | | | converting enzyme cDNA. |
| protein | GENESEQ_PROT\|AAR92098 | GENESEQ_PROT\|AAR92098 | Beta-ionone 4-methylene gp. to keto gp. |
| | | | converting enzyme cDNA. |
| dna | GENESEQ_PROT\|AAR92099 | unknown id | Beta-ionone 4-methylene gp. to keto gp. |
| | | | converting enzyme cDNA. |
| protein | GENESEQ_PROT\|AAR92099 | GENESEQ_PROT\|AAR92099 | Beta-ionone 4-methylene gp. to keto gp. |
| | | | converting enzyme cDNA. |
| dna | GENESEQ_PROT\|AAW98198 | GENESEQ_DNA\|AAX25068 | SSU/crtW (beta-carotene ketolase) gene fusion. |
| protein | GENESEQ_PROT\|AAW98198 | GENESEQ_PROT\|AAW98198 | SSU/(beta-carotene ketolase fusion. |
| dna | GENESEQ_PROT\|ABU97244 | GENESEQ_DNA\|ACA99471 | DNA encoding enzyme polypeptide #10. |
| protein | GENESEQ_PROT\|ABU97244 | GENESEQ_PROT\|ABU97244 | Enzyme polypeptide #10. |
| dna | GENESEQ_PROT\|ADP74106 | unknown id | Nostoc punctiforme ketolase DNA SEQ ID |
| | | | NO 3 variant #1. |
| protein | GENESEQ_PROT\|ADP74106 | GENESEQ_PROT\|ADP74106 | Nostoc punctiforme ketolase DNA SEQ ID |
| | | | NO 3 variant #1. |
| dna | GENESEQ_PROT\|ADP74108 | unknown id | Nostoc punctiforme ketolase DNA SEQ ID |
| | | | NO 3 variant #2. |
| protein | GENESEQ_PROT\|ADP74108 | GENESEQ_PROT\|ADP74108 | Nostoc punctiforme ketolase DNA SEQ ID |
| | | | NO 3 variant #2. |
| dna | GENESEQ_PROT\|ADP74110 | unknown id | Nostoc punctiforme ketolase DNA SEQ ID |
| | | | NO 5 variant #1. |
| protein | GENESEQ_PROT\|ADP74110 | GENESEQ_PROT\|ADP74110 | Nostoc punctiforme ketolase DNA SEQ ID |
| | | | NO 5 variant #1. |
| dna | GENESEQ_PROT\|ADP74112 | unknown id | Nostoc punctiforme ketolase DNA SEQ ID |
| | | | NO 5 variant #2. |
| protein | GENESEQ_PROT\|ADP74112 | GENESEQ_PROT\|ADP74112 | Nostoc punctiforme ketolase DNA SEQ ID |
| | | | NO 5 variant #2. |
| dna | GENESEQ_PROT\|ADP74147 | unknown id | Synechococcus sp. WH 8102 ketolase variant |
| | | | DNA #1. |
| protein | GENESEQ_PROT\|ADP74147 | GENESEQ_PROT\|ADP74147 | Synechococcus sp. WH 8102 ketolase variant |
| | | | DNA #1. |
| dna | GENESEQ_PROT\|ADP74149 | unknown id | Synechococcus sp. WH 8102 ketolase variant |
| | | | DNA #2. |
| protein | GENESEQ_PROT\|ADP74149 | GENESEQ_PROT\|ADP74149 | Synechococcus sp. WH 8102 ketolase variant |
| | | | DNA #2. |
| dna | GENESEQ_PROT\|ADQ38260 | unknown id | H. pluvialis ketolase DNA. |
| protein | GENESEQ_PROT\|ADQ38260 | GENESEQ_PROT\|ADQ38260 | H. pluvialis ketolase DNA. |
| dna | GENESEQ_PROT\|ADQ38262 | unknown id | H. pluvialis ketolase DNA. |
| protein | GENESEQ_PROT\|ADQ38262 | GENESEQ_PROT\|ADQ38262 | H. pluvialis ketolase DNA. |
| dna | GENESEQ_PROT\|ADQ38264 | unknown id | H. pluvialis ketolase DNA. |
| protein | GENESEQ_PROT\|ADQ38264 | GENESEQ_PROT\|ADQ38264 | H. pluvialis ketolase DNA. |
| dna | GENESEQ_PROT\|ADQ38323 | unknown id | Synechocystis sp. WH 8102 ketolase DNA. |
| protein | GENESEQ_PROT\|ADQ38323 | GENESEQ_PROT\|ADQ38323 | Synechocystis sp. WH 8102 ketolase DNA. |
| dna | GENESEQ_PROT\|ADQ96834 | unknown id | CrtWcrtY nucleotide sequence. |
| protein | GENESEQ_PROT\|ADQ96834 | GENESEQ_PROT\|ADQ96834 | CrtWcrtY nucleotide sequence. |
| dna | GENESEQ_PROT\|ADR03950 | unknown id | Nostoc punctiforme ketolase coding sequence |
| | | | #1. |
| protein | GENESEQ_PROT\|ADR03950 | GENESEQ_PROT\|ADR03950 | “no start codon” |
| dna | GENESEQ_PROT\|ADR03952 | unknown id | Nostoc punctiforme ketolase coding sequence |
| | | | #2. |
| protein | GENESEQ_PROT\|ADR03952 | GENESEQ_PROT\|ADR03952 | “no start codon” |
| dna | GENESEQ_PROT\|ADY51414 | unknown id | Nodularia spumigena NODK ketolase DNA. |
| protein | GENESEQ_PROT\|ADY51414 | GENESEQ_PROT\|ADY51414 | Nodularia spumigena NODK ketolase DNA. |
| dna | GENESEQ_PROT\|ADY52469 | GENESEQ_DNA\|ADY51401 | Nostoc punctiforme ketolase NP196 DNA. |
| protein | GENESEQ_PROT\|ADY52469 | GENESEQ_PROT\|ADY52469 | Novel ketocarotenoid preparation method- |
| | | | related protein SeqID103. |
| dna | GENESEQ_PROT\|ADY52906 | GENESEQ_DNA\|ADY52905 | N. spumigena strain NSOR10 ketocarotenoid- |
| | | | related putative ketolase DNA. |
| protein | GENESEQ_PROT\|ADY52906 | GENESEQ_PROT\|ADY52906 | N. spumigena NSOR10 ketocarotenoid- |
| | | | related putative ketolase protein. |
| dna | GENESEQ_PROT\|ADY52908 | GENESEQ_DNA\|ADY52907 | Nodularia spumigena ketocarotenoid-related |
| | | | ketolase DNA - SEQ ID 3. |
| protein | GENESEQ_PROT\|ADY52908 | GENESEQ_PROT\|ADY52908 | Nodularia spumigena ketocarotenoid-related |
| | | | ketolase protein - SEQ ID 4. |
| dna | GENESEQ_PROT\|ADY52910 | GENESEQ_DNA\|ADY52911 | Nodularia spumigena ketocarotenoid-related |
| | | | ketolase DNA - SEQ ID 7. |
| protein | GENESEQ_PROT\|ADY52910 | GENESEQ_PROT\|ADY52910 | Nodularia spumigena ketocarotenoid-related |
| | | | ketolase protein - SEQ ID 6. |
| dna | GENESEQ_PROT\|ADY52914 | GENESEQ_DNA\|ADY52913 | Nostoc punctiforme ketocarotenoid-related |
| | | | ketolase DNA - SEQ ID 9. |
| protein | GENESEQ_PROT\|ADY52914 | GENESEQ_PROT\|ADY52914 | Nostoc punctiforme ketocarotenoid-related |
| | | | ketolase protein - SEQ ID 10. |
| dna | GENESEQ_PROT\|ADY52916 | GENESEQ_DNA\|ADY52915 | Nostoc punctiforme ketocarotenoid-related |
| | | | ketolase DNA - SEQ ID 11. |
| protein | GENESEQ_PROT\|ADY52916 | GENESEQ_PROT\|ADY52916 | Nostoc punctiforme ketocarotenoid-related |
| | | | ketolase protein - SEQ ID 12. |
| dna | GENESEQ_PROT\|ADY52966 | GENESEQ_DNA\|ADY52965 | Nostoc punctiforme ketocarotenoid-related |
| | | | ketolase DNA - SEQ ID 61. |
| protein | GENESEQ_PROT\|ADY52966 | GENESEQ_PROT\|ADY52966 | Nostoc punctiforme ketocarotenoid-related |
| | | | ketolase protein - SEQ ID 62. |
| dna | GENESEQ_PROT\|ADY52971 | GENESEQ_DNA\|ADY52970 | Nostoc punctiforme ketocarotenoid-related |
| | | | ketolase DNA - SEQ ID 66. |
| protein | GENESEQ_PROT\|ADY52971 | GENESEQ_PROT\|ADY52971 | Nostoc punctiforme ketocarotenoid-related |
| | | | ketolase protein - SEQ ID 67. |
| dna | GENESEQ_PROT\|ADY52974 | GENESEQ_DNA\|ADY52973 | Nodularia spumigena ketocarotenoid-related |
| | | | ketolase DNA - SEQ ID 69. |
| protein | GENESEQ_PROT\|ADY52974 | GENESEQ_PROT\|ADY52974 | Nodularia spumigena ketocarotenoid-related |
| | | | ketolase protein - SEQ ID 70. |
| dna | GENESEQ_PROT\|ADY52976 | GENESEQ_DNA\|ADY52975 | Nodularia spumigena ketocarotenoid-related |
| | | | ketolase DNA - SEQ ID 71. |
| protein | GENESEQ_PROT\|ADY52976 | GENESEQ_PROT\|ADY52976 | Nodularia spumigena ketocarotenoid-related |
| | | | ketolase protein - SEQ ID 72. |
| dna | GENESEQ_PROT\|ADY52980 | GENESEQ_DNA\|ADY52917 | Gloeobacter violaceus ketocarotenoid-related |
| | | | ketolase DNA - SEQ ID 13. |
| protein | GENESEQ_PROT\|ADY52980 | GENESEQ_PROT\|ADY52980 | Gloeobacter violaceus ketocarotenoid-related |
| | | | ketolase protein - SEQ 76. |
| dna | GENESEQ_PROT\|AEB92197 | unknown id | S. melonis beta-carotene C4 oxygenase |
| | | | DNA. |
| protein | GENESEQ_PROT\|AEB92197 | GENESEQ_PROT\|AEB92197 | S. melonis beta-carotene C4 oxygenase |
| | | | DNA. |
| dna | GENESEQ_PROT\|AEB92199 | unknown id | B. vesicularis beta-carotene C4 oxygenase |
| | | | DNA. |
| protein | GENESEQ_PROT\|AEB92199 | GENESEQ_PROT\|AEB92199 | B. vesicularis beta-carotene C4 oxygenase |
| | | | DNA. |
| dna | GENESEQ_PROT\|AEK41647 | GENESEQ_DNA\|AEK41646 | Carotenoid biosynthetic pathway associated |
| | | | DNA, SEQ ID NO: 17. |
| protein | GENESEQ_PROT\|AEK41647 | GENESEQ_PROT\|AEK41647 | Carotenoid biosynthetic pathway associated |
| | | | enzyme, SEQ ID NO: 18. |
| dna | GENESEQ_PROT\|AEK41703 | GENESEQ_DNA\|AEK41702 | Carotenoid biosynthetic pathway associated |
| | | | DNA, SEQ ID NO: 73. |
| protein | GENESEQ_PROT\|AEK41703 | GENESEQ_PROT\|AEK41703 | Carotenoid biosynthetic pathway associated |
| | | | enzyme, SEQ ID NO: 74. |
| dna | GENESEQ_PROT\|AEK41725 | GENESEQ_DNA\|AEK41724 | Carotenoid biosynthetic pathway associated |
| | | | DNA, SEQ ID NO: 95. |
| protein | GENESEQ_PROT\|AEK41725 | GENESEQ_PROT\|AEK41725 | Carotenoid biosynthetic pathway associated |
| | | | enzyme, SEQ ID NO: 96. |
| dna | GENESEQ_PROT\|AEK41826 | GENESEQ_DNA\|AEK41686 | Carotenoid biosynthetic pathway associated |
| | | | DNA, SEQ ID NO: 57. |
| protein | GENESEQ_PROT\|AEK41826 | GENESEQ_PROT\|AEK41826 | Astaxanthin biosynthetic pathway vector |
| | | | related proein, SEQ ID NO: 197. |
| dna | GENESEQ_PROT\|AEL17450 | unknown id | Carotene ketolase gene. |
| protein | GENESEQ_PROT\|AEL17450 | GENESEQ_VPROT\|AEL17450 | Carotene ketolase gene. |
| dna | REFSEQ_PROTEIN\|NP_108024 | REFSEQ_NUCLEOTIDE\|NC_002678 | Mesorhizobium loti MAFF303099, complete |
| | | | genome. |
| protein | REFSEQ_PROTEIN\|NP_108024 | REFSEQ_PROTEIN\|NP_108024 | similar to Rhizopine catabolism protein |
| | | | mocD [Mesorhizobium loti MAFF303099]. |
| dna | REFSEQ_PROTEIN\|NP_487229 | REFSEQ_NUCLEOTIDE\|NC_003272 | Nostoc sp. PCC 7120, complete genome. |
| protein | REFSEQ_PROTEIN\|NP_487229 | REFSEQ_PROTEIN\|NP_487229 | beta-carotene ketolase [Nostoc sp. PCC |
| | | | 7120]. |
| dna | REFSEQ_PROTEIN\|NP_897461 | REFSEQ_NUCLEOTIDE\|NC_005070 | Synechococcus sp. WH 8102, complete |
| | | | genome. |
| protein | REFSEQ_PROTEIN\|NP_897461 | REFSEQ_PROTEIN\|NP_897461 | possible beta-carotene ketolase |
| | | | [Synechococcus sp. WH 8102]. |
| dna | REFSEQ_PROTEIN\|NP_924674 | REFSEQ_NUCLEOTIDE\|NC_005125 | Gloeobacter violaceus PCC 7421, complete |
| | | | genome. |
| protein | REFSEQ_PROTEIN\|NP_924674 | REFSEQ_PROTEIN\|NP_924674 | beta-carotene ketolase [Gloeobacter |
| | | | violaceus PCC 7421]. |
| dna | REFSEQ_PROTEIN\|YP_322565 | REFSEQ_NUCLEOTIDE\|NC_007413 | Anabaena variabilis ATCC 29413, complete |
| | | | genome. |
| protein | REFSEQ_PROTEIN\|YP_322565 | REFSEQ_PROTEIN\|YP_322565 | Fatty acid desaturase [Anabaena variabilis |
| | | | ATCC 29413]. |
| dna | REFSEQ_PROTEIN\|YP_324388 | REFSEQ_NUCLEOTIDE\|NC_007413 | Anabaena variabilis ATCC 29413, complete |
| | | | genome. |
| protein | REFSEQ_PROTEIN\|YP_324388 | REFSEQ_PROTEIN\|YP_324388 | Fatty acid desaturase [Anabaena variabilis |
| | | | ATCC 29413]. |
| dna | REFSEQ_PROTEIN\|YP_376982 | REFSEQ_NUCLEOTIDE\|NC_007513 | Synechococcus sp. CC9902, complete |
| | | | genome. |
| protein | REFSEQ_PROTEIN\|YP_376982 | REFSEQ_PROTEIN\|YP_376982 | possible beta-carotene ketolase |
| | | | [Synechococcus sp. CC9902]. |
| dna | REFSEQ_PROTEIN\|YP_457553 | REFSEQ_NUCLEOTIDE\|NC_007722 | Erythrobacter litoralis HTCC2594, complete |
| | | | genome. |
| protein | REFSEQ_PROTEIN\|YP_457553 | REFSEQ_PROTEIN\|YP_457553 | beta-carotene ketolase [Erythrobacter |
| | | | litoralis HTCC2594]. |
| dna | REFSEQ_PROTEIN\|YP_475340 | REFSEQ_NUCLEOTIDE\|NC_007775 | Synechococcus sp. JA-3-3Ab, complete |
| | | | genome. |
| protein | REFSEQ_PROTEIN\|YP_475340 | REFSEQ_PROTEIN\|YP_475340 | fatty acid desaturase [Synechococcus sp. |
| | | | JA-3-3Ab]. |
| dna | REFSEQ_PROTEIN\|YP_476366 | REFSEQ_NUCLEOTIDE\|NC_007776 | Synechococcus sp. JA-2-3B′a(2-13), |
| | | | complete genome. |
| protein | REFSEQ_PROTEIN\|YP_476366 | REFSEQ_PROTEIN\|YP_476366 | beta-carotene ketolase, putative |
| | | | [Synechococcus sp. JA-2-3B′a(2-13)]. |
| dna | REFSEQ_PROTEIN\|YP_634097 | REFSEQ_NUCLEOTIDE\|NC_008095 | Myxococcus xanthus DK 1622, complete |
| | | | genome. |
| protein | REFSEQ_PROTEIN\|YP_634097 | REFSEQ_PROTEIN\|YP_634097 | fatty acid desaturase family protein |
| | | | [Myxococcus xanthus DK 1622]. |
| dna | REFSEQ_PROTEIN\|YP_634184 | REFSEQ_NUCLEOTIDE\|NC_008095 | Myxococcus xanthus DK 1622, complete |
| | | | genome. |
| protein | REFSEQ_PROTEIN\|YP_634184 | REFSEQ_PROTEIN\|YP_634184 | fatty acid desaturase family protein |
| | | | [Myxococcus xanthus DK 1622]. |
| dna | REFSEQ_PROTEIN\|YP_731008 | REFSEQ_NUCLEOTIDE\|NC_008319 | Synechococcus sp. CC9311, complete |
| | | | genome. |
| protein | REFSEQ_PROTEIN\|YP_731008 | REFSEQ_PROTEIN\|YP_731008 | possible beta-carotene ketolase |
| | | | [Synechococcus sp. CC9311]. |
| dna | REFSEQ_PROTEIN\|ZP_00111258 | REFSEQ_NUCLEOTIDE\|NZ_AAAY02000007 | Nostoc punctiforme PCC 73102, unfinished |
| | | | sequence, whole genome shotgun sequence. |
| protein | REFSEQ_PROTEIN\|ZP_00111258 | REFSEQ_PROTEIN\|ZP_00111258 | COG3239: Fatty acid desaturase [Nostoc |
| | | | punctiforme PCC 73102]. |
| dna | REFSEQ_PROTEIN\|ZP_00345866 | GB_patent\|DD399711 | Method for producing carotenoids or their |
| | | | precursors using genetically modified |
| | | | organisms of the Blakeslea genus, |
| | | | carotenoids or their precursors produced by |
| | | | said method and use thereof. |
| protein | REFSEQ_PROTEIN\|ZP_00345866 | REFSEQ_PROTEIN\|ZP_00345866 | hypothetical protein Npun02000865 [Nostoc |
| | | | punctiforme PCC 73102]. |
| dna | REFSEQ_PROTEIN\|ZP_00514501 | REFSEQ_NUCLEOTIDE\|NZ_AADV02000002 | Crocosphaera watsonii WH 8501, unfinished |
| | | | sequence, whole genome shotgun sequence. |
| protein | REFSEQ_PROTEIN\|ZP_00514501 | REFSEQ_PROTEIN\|ZP_00514501 | Fatty acid desaturase [Crocosphaera |
| | | | watsonii WH 8501]. |
| dna | REFSEQ_PROTEIN\|ZP_01018056 | REFSEQ_NUCLEOTIDE\|NZ_AAMU01000003 | Parvularcula bermudensis HTCC2503, |
| | | | unfinished sequence, whole genome shotgun |
| | | | sequence. |
| protein | REFSEQ_PROTEIN\|ZP_01018056 | REFSEQ_PROTEIN\|ZP_01018056 | Fatty acid desaturase [Parvularcula |
| | | | bermudensis HTCC2503]. |
| dna | REFSEQ_PROTEIN\|ZP_01041617 | REFSEQ_NUCLEOTIDE\|NZ_AAMW01000003 | Erythrobacter sp. NAP1, unfinished |
| | | | sequence, whole genome shotgun sequence. |
| protein | REFSEQ_PROTEIN\|ZP_01041617 | REFSEQ_PROTEIN\|ZP_01041617 | Fatty acid desaturase [Erythrobacter sp. |
| | | | NAP1]. |
| dna | REFSEQ_PROTEIN\|ZP_01080541 | REFSEQ_NUCLEOTIDE\|NZ_AANP01000004 | Synechococcus sp. RS9917, unfinished |
| | | | sequence, whole genome shotgun sequence. |
| protein | REFSEQ_PROTEIN\|ZP_01080541 | REFSEQ_PROTEIN\|ZP_01080541 | possible beta-carotene ketolase |
| | | | [Synechococcus sp. RS9917]. |
| dna | REFSEQ_PROTEIN\|ZP_01083421 | REFSEQ_NUCLEOTIDE\|NZ_AANO01000001 | Synechococcus sp. WH 5701, unfinished |
| | | | sequence, whole genome shotgun sequence. |
| protein | REFSEQ_PROTEIN\|ZP_01083421 | REFSEQ_PROTEIN\|ZP_01083421 | possible beta-carotene ketolase |
| | | | [Synechococcus sp. WH 5701]. |
| dna | REFSEQ_PROTEIN\|ZP_01123773 | REFSEQ_NUCLEOTIDE\|NZ_AAOK01000003 | Synechococcus sp. WH 7805, unfinished |
| | | | sequence, whole genome shotgun sequence. |
| protein | REFSEQ_PROTEIN\|ZP_01123773 | REFSEQ_PROTEIN\|ZP_01123773 | possible beta-carotene ketolase |
| | | | [Synechococcus sp. WH 7805]. |
| dna | REFSEQ_PROTEIN\|ZP_01226542 | REFSEQ_NUCLEOTIDE\|NZ_AAPJ01000002 | Aurantimonas sp. SI85-9A1, unfinished |
| | | | sequence, whole genome shotgun sequence. |
| protein | REFSEQ_PROTEIN\|ZP_01226542 | REFSEQ_PROTEIN\|ZP_01226542 | beta-carotene ketolase/oxygenase |
| | | | [Aurantimonas sp. SI85-9A1]. |
| dna | REFSEQ_PROTEIN\|ZP_01439496 | REFSEQ_NUCLEOTIDE\|NZ_AATP01000004 | Fulvimarina pelagi HTCC2506, unfinished |
| | | | sequence, whole genome shotgun sequence. |
| protein | REFSEQ_PROTEIN\|ZP_01439496 | REFSEQ_PROTEIN\|ZP_01439496 | beta-carotene ketolase [Fulvimarina pelagi |
| | | | HTCC2506]. |
| dna | REFSEQ_PROTEIN\|ZP_01468055 | REFSEQ_NUCLEOTIDE\|NZ_AATZ01000001 | Synechococcus sp. BL107, unfinished |
| | | | sequence, whole genome shotgun sequence. |
| protein | REFSEQ_PROTEIN\|ZP_01468055 | REFSEQ_PROTEIN\|ZP_01468055 | possible beta-carotene ketolase |
| | | | [Synechococcus sp. BL107]. |
| dna | REFSEQ_PROTEIN\|ZP_01632305 | REFSEQ_NUCLEOTIDE\|NZ_AAVW01000111 | Nodularia spumigena CCY9414, unfinished |
| | | | sequence, whole genome shotgun sequence. |
| protein | REFSEQ_PROTEIN\|ZP_01632305 | REFSEQ_PROTEIN\|ZP_01632305 | Fatty acid desaturase [Nodularia spumigena |
| | | | CCY9414]. |
| dna | REFSEQ_PROTEIN\|ZP_01632726 | REFSEQ_NUCLEOTIDE\|NZ_AAVW01000162 | Nodularia spumigena CCY9414, unfinished |
| | | | sequence, whole genome shotgun sequence. |
| protein | REFSEQ_PROTEIN\|ZP_01632726 | REFSEQ_PROTEIN\|ZP_01632726 | Fatty acid desaturase [Nodularia spumigena |
| | | | CCY9414]. |
| dna | SPTREMBL\|O23973_HAEPL | GB_plant\|X86782 | H. pluvialis mRNA for beta-carotene C-4 |
| | | | oxygenase. |
| protein | SPTREMBL\|O23973_HAEPL | SPTREMBL\|O23973_HAEPL | Beta-carotene C-4 oxygenase (Ketolase). |
| dna | SPTREMBL\|Q0H2C9_BREVE | GB_bacterial\|DQ309446 | Brevundimonas vesicularis carotenoid |
| | | | synthesis gene cluster, complete sequence. |
| protein | SPTREMBL\|Q0H2C9_BREVE | SPTREMBL\|Q0H2C9_BREVE | Beta-carotene ketolase. |
| protein | SPTREMBL\|Q15I90_9SPHI | SPTREMBL\|Q15I90_9SPHI | CrtW. |
| dna | SPTREMBL\|Q24K64_9RHOB | GB_bacterial\|AY957386 | Paracoccus haeundaensis astaxanthin |
| | | | biosynthesis gene cluster, complete sequence. |
| protein | SPTREMBL\|Q24K64_9RHOB | SPTREMBL\|Q24K64_9RHOB | Beta-carotene ketolase. |
| dna | SPTREMBL\|Q2XST9_HAEPL | GB_plant\|DQ257290 | Haematococcus pluvialis 34-1n beta-carotene |
| | | | ketolase (crtO) mRNA, partial cds. |
| protein | SPTREMBL\|Q2XST9_HAEPL | SPTREMBL\|Q2XST9_HAEPL | Beta-carotene ketolase (Fragment). |
| protein | SPTREMBL\|Q4FE71_HAEPL | SPTREMBL\|Q4FE71_HAEPL | Beta-carotene C-4 oxygenase. |
| dna | SPTREMBL\|Q4VKB4_CHLRE | GB_plant\|AY860820 | Chlamydomonas reinhardtii putative |
| | | | chloroplast carotene beta ketolase precursor |
| | | | (BKT) mRNA, complete cds; nuclear gene |
| | | | for chloroplast product. |
| protein | SPTREMBL\|Q4VKB4_CHLRE | SPTREMBL\|Q4VKB4_CHLRE | Putative chloroplast carotene beta ketolase. |
| protein | SPTREMBL\|Q4W8B8_9CAUL | SPTREMBL\|Q4W8B8_9CAUL | Beta-carotene ketolase. |
| protein | SPTREMBL\|Q5U931_9CHLO | SPTREMBL\|Q5U931_9CHLO | Beta-carotene ketolase/oxygenase. |
| dna | SPTREMBL\|Q6J3N5_HAEPL | GB_plant\|AY603347 | Haematococcus pluvialis strain NIES-144 |
| | | | beta-carotene ketolase (bkt3) mRNA, |
| | | | complete cds. |
| protein | SPTREMBL\|Q6J3N5_HAEPL | SPTREMBL\|Q6J3N5_HAEPL | Beta-carotene ketolase. |
| dna | SPTREMBL\|Q847D1_NODSP | GB_patent\|CS050451 | Sequence 37 from Patent WO2005019460. |
| protein | SPTREMBL\|Q847D1_NODSP | SPTREMBL\|Q847D1_NODSP | Putative beta-carotene ketolase. |
| dna | SPTREMBL\|Q8GCT5_9CAUL | GB_bacterial\|AY166610 | Brevundimonas aurantiaca beta-carotene C4 |
| | | | oxygenase (crtW) gene, complete cds. |
| protein | SPTREMBL\|Q8GCT5_9CAUL | SPTREMBL\|Q8GCT5_9CAUL | Beta-carotene C4 oxygenase. |
| dna | SPTREMBL\|Q8LJQ2_HAEPL | GB_patent\|CS050445 | Sequence 31 from Patent WO2005019460. |
| protein | SPTREMBL\|Q8LJQ2_HAEPL | SPTREMBL\|Q8LJQ2_HAEPL | BKT. |
| dna | SPTREMBL\|Q9KIX0_9BRAD | GB_patent\|DD399668 | Method for producing carotenoids or their |
| | | | precursors using genetically modified |
| | | | organisms of the Blakeslea genus, |
| | | | carotenoids or their precursors produced by |
| | | | said method and use thereof. |
| protein | SPTREMBL\|Q9KIX0_9BRAD | SPTREMBL\|Q9KIX0_9BRAD | Beta-carotene ketolase. |
| dna | SPTREMBL\|Q9RLH7_9RHOB | GB_patent\|DD399666 | Method for producing carotenoids or their |
| | | | precursors using genetically modified |
| | | | organisms of the Blakeslea genus, |
| | | | carotenoids or their precursors produced by |
| | | | said method and use thereof. |
| protein | SPTREMBL\|Q9RLH7_9RHOB | SPTREMBL\|Q9RLH7_9RHOB | Beta-carotene C-4-oxygenase (Ketolase). |
| dna | SWISSPROT\|CRTW_HAEPL | GB_patent\|DD367504 | Peptides capable of transiting to chromoplast |
| | | | and a method for producing a plant having |
| | | | yellowish petals using the peptides. |
| protein | SWISSPROT\|CRTW_HAEPL | SWISSPROT\|CRTW_HAEPL | Beta-carotene ketolase (EC 1.13.—.—) |
| | | | (Beta-carotene oxygenase). |
| protein | SWISSPROT\|CRTW_PARS1 | SWISSPROT\|CRTW_PARS1 | Beta-carotene ketolase (EC 1.13.—.—) |
| | | | (Beta-carotene oxygenase). |
| protein | SWISSPROT\|CRTW_PARSN | SWISSPROT\|CRTW_PARSN | Beta-carotene ketolase (EC 1.13.—.—) |
| | | | (Beta-carotene oxygenase). |

Annex 2
Beta-Cyclase

| Species | Protein | DNA |
|---|---|---|
| Lycopersicon esculentum | AAG21133 | CQ788417 |
| Lycopersicon esculentum | Q43503 | ADQ38256 |
| Adonis aestivalis var | Q9AXL1_9MAGN | missing |
| Ananas comosus | AEM23455 | AEM23454 |
| arabidopsis thaliana | SL000005.67999 | US20040034888A1.14372 |
| arabidopsis thaliana | SL000005.68000 | US20040034888A1.14372 |
| arabidopsis thaliana | US20040034888A1.40728 | US20040034888A1.14372 |
| Arabidopsis thaliana | ADO05224 | ADO05147 |
| Arabidopsis thaliana | LCYB_ARATH | AY091396 |
| Bixa orellana | Q70SZ9_BIXOR | AJ549288 |
| Capsicum annuum | CCS_CAPAN | DD367511 |
| Capsicum annuum | LCYB_CAPAN | X86221 |
| Capsicum sp | AAY54313 | missing |
| Carica papaya | Q1WAB6_CARPA | DQ415894 |
| Chlamydomonas reinhardtii | Q4VKB6_CHLRE | AY860818 |
| Chrysanthemum x morifolium | Q2HXK5_9ASTR | AB205041 |
| Citrullus lanatus | A0FJC0_CITLA | EF014290 |
| Citrullus lanatus | A0FJC0_CITLA | EF014290 |
| Citrullus lanatus | A2TDC8_CITLA | EF183521 |
| Citrullus lanatus | A2TDC9_CITLA | EF183522 |
| Citrus clementina | Q6B839_9ROSI | AY675216 |
| Citrus limon | Q766E1_CITLI | AB114668 |
| Citrus maxima | Q6XQ45_CITMA | AY217103 |
| Citrus paradisi | Q9XGX3_CITPA | AF152246 |
| Citrus sinensis | Q19QX8_CITSI | DQ496224 |
| Citrus sinensis | Q2I740_CITSI | DQ235259 |
| Citrus sinensis | Q64HC6_CITSI | AY644699 |
| Citrus sinensis | Q66NC9_CITSI | AY679168 |
| Citrus sinensis | Q66ND0_CITSI | AY679167 |
| Citrus sinensis | Q766E9_CITSI | AB114660 |
| Citrus sinensis | Q8LPP7_CITSI | AY094582 |
| Citrus sinensis | Q9M546_CITSI | AF240787 |
| Citrus sinensis | CCS_CITSI | AF169241 |
| Citrus unshiu | Q8GTR2_CITUN | AY166796 |
| Citrus x paradisi | ADO05211 | missing |
| Cryptomeria japonica | Q403R7_CRYJA | AB161847 |
| Cryptomeria japonica | Q403R8_CRYJA | AB161846 |
| Cryptomeria japonica | Q403R9_CRYJA | AB161845 |
| Cryptomeria japonica | Q403S0_CRYJA | AB161844 |
| Cryptomeria japonica | Q403S2_CRYJA | AB161843 |
| Cryptomeria japonica | Q403S4_CRYJA | AB161840 |
| Cryptomeria japonica | Q403S8_CRYJA | AB161836 |
| Cryptomeria japonica | Q403T3_CRYJA | AB161831 |
| Cryptomeria japonica | Q403T4_CRYJA | AB161830 |
| Cryptomeria japonica | Q403T5_CRYJA | AB161829 |
| Cryptomeria japonica | Q403T9_CRYJA | AB161825 |
| Cryptomeria japonica | Q403U2_CRYJA | AB161822 |
| Cryptomeria japonica | Q403U4_CRYJA | AB161820 |
| Cryptomeria japonica | Q403U5_CRYJA | AB161837 |
| Cryptomeria japonica | Q76IW9_CRYJA | AB096553 |
| Cryptomeria japonica | Q76IZ0_CRYJA | AB161817 |
| Cryptomeria japonica | Q76IZ4_CRYJA | AB096525 |
| Cryptomeria japonica | Q76IZ6_CRYJA | AB096557 |
| Cryptomeria japonica | Q76J03_CRYJA | AB161838 |
| Cryptomeria japonica | Q76J04_CRYJA | AB096549 |
| Cryptomeria japonica | Q76J08_CRYJA | AB161841 |
| Daucus carota subsp | Q2VEX6_DAUCA | missing |
| Daucus carota subsp | Q2VEX7_DAUCA | missing |
| Gentiana lutea | A0SY32_GENLU | EF062505 |
| Gentiana lutea | Q1XIT7_GENLU | AB017367 |
| Gentiana lutea | Q1XIT8_GENLU | AB017366 |
| glycine max | US20040031072A1.272683 | US20040031072A1.129841 |
| glycine max | US20040034888A1.52568 | US20040034888A1.14874 |
| Glycine max | AAY32325 | AAZ34973 |
| Haematococcus pluvialis | Q330P8_HAEPL | AY182008 |
| Hordeum vulgare | ABM74275 | missing |
| Lycium barbarum | Q5ECK9_LYCBA | AY906864 |
| Lycopersicon pennellii | AAY70397 | AAZ51519 |
| Medicago truncatula | A2Q2Y6_MEDTR | AC153128 |
| Narcissus pseudonarcissus | LCYB_NARPS | X98796 |
| Nicotiana langsdorffii x Nicotiana sanderae | Q078Z0_NICLS | DQ212774 |
| Nicotiana sp | AAY54315 | missing |
| Nicotiana tabacum | LCYB_TOBAC | X81787 |
| Oryza sativa | Q6YUU2_ORYSA | NM_001052686 |
| Oryza sativa (japonica cultivar-group) | NP_001046151 | NM_001052686 |
| Ostreococcus tauri | Q00WD1_OSTTA | missing |
| Salicornia europaea | A1KYX9_SALEU | AY789516 |
| Sandersonia aurantiaca | Q8S3C3_SANAU | AF489520 |
| Setaria italica | Q7XAV8_SETIT | AY337024 |
| Solanum lycopersicum | Q9FV32_SOLLC | CQ788417 |
| Solanum lycopersicum | Q9LWA6_SOLLC | Y18297 |
| Solanum lycopersicum | LCYB_SOLLC | CQ793497 |
| Solanum tuberosum | Q9M424_SOLTU | CS422390 |
| Synthetic | AAY54289 | missing |
| Tagetes erecta | AAY90226 | AAA07582 |
| Tagetes erecta | Q8L8H5_TARER | AY099484 |
| Tagetes erecta | Q9FV42_TARER | AF251017 |
| Taraxacum officinale | Q2MGW7_TAROF | AB247456 |
| Taxodium distichum | Q76IR1_TAXDI | AB161848 |
| Taxodium distichum var | Q403Q1_TAXDI | missing |
| Taxodium distichum var | Q403Q2_TAXDI | missing |
| Taxodium distichum var | Q403Q3_TAXDI | missing |
| Taxodium distichum var | Q403Q4_TAXDI | missing |
| Taxodium distichum var | Q403Q5_TAXDI | missing |
| Taxodium distichum var | Q403Q6_TAXDI | missing |
| Taxodium distichum var | Q403Q7_TAXDI | missing |
| Taxodium distichum var | Q403Q8_TAXDI | missing |
| Taxodium distichum var | Q403Q9_TAXDI | missing |
| Taxodium distichum var | Q403R0_TAXDI | missing |
| Taxodium distichum var | Q403R1_TAXDI | missing |
| Taxodium distichum var | Q403R2_TAXDI | missing |
| Taxodium distichum var | Q403R3_TAXDI | missing |
| Taxodium distichum var | Q403R4_TAXDI | missing |
| Taxodium distichum var | Q403R5_TAXDI | missing |
| Unidentified | ABG93898 | missing |
| zea mays | US20040214272A1.237207 | US20040214272A1.52544 |
| Zea mays | US2004214272.237207 | missing |
| Zea mays | AAY32324 | AAZ34972 |
| Zea mays | Q84VG9_MAIZE | AY206862 |

1. A process for the preparation of at least one carotenoid in a genetically modified plant, which method comprises expressing in a plant at least one carotene ketolase enzyme (K-enzyme) encoded by an expression improved heterologous carotene ketolase coding sequence (Ki-sequence).

2. The process of claim 1, wherein
(a) the at least one K-enzyme encoded by a Ki-sequence (Ki-enzyme) is coexpressed with at least one beta-cyclase enzyme (Bc-enzyme) encoded by a Bc-sequence;
(b) at least one of the carotenoids is a ketocarotenoid;
(c) the plant is from the genus Tagetes or any of the following families: Ranunculaceae, Berberidaceae, Begoniaceae, Papaveraceae, Cannabaceae, Chenopodiaceae, Cruciferae, Rosaceae, Fabaceae, Linaceae, Vitaceae, Brassiceae, Cucurbitaceae, Primulaceae, Caryophyllaceae, Amaranthaceae, Apocynaceae, Balsaminaceae, Gentianaceae, Geraniaceae, Graminae, Euphorbiaceae, Labiatae, Leguminosae, Caprifoliaceae, Oleaceae, Tropaeolaceae, Solanaceae, Lobeliaceae, Scrophulariaceae, Compositae, Asteraceae, Plumbaginaceae, Liliaceae, Amaryllidaceae, Rubiaceae, Poaceae, Polemoniaceae, Orchidaceae, Umbelliferae, Verbenaceae, Violaceae, Malvaceae, Illiaceae or Lamiaceae;
(d) the Ki-sequence is derived from the corresponding coding sequence from bacteria, yeast or algae;
(e) the Ki-sequence is selected from the corresponding coding portions comprised by a nucleotide sequence selected from SEQ ID NO: 3, 4, 7, 8, 10, 11, 13 and 14; or coding sequences derived therefrom by nucleic acid substitution, addition, deletion or insertion, and encoding a ketolase enzyme having a sequence identity of at least 50% with respect to the parent sequence and retaining ketolase activity; or
(f) the K-enzyme is expressed having an amino acid sequence encoded by the corresponding coding portions comprised by a nucleotide sequence selected from SEQ ID NO: 3, 4, 7, 8, 10, 11, 13 and 14; or coding sequences derived therefrom by nucleic acid substitution, addition, deletion or insertion, and encoding a ketolase enzyme having a sequence identity of at least 50% with respect to the parent sequence and retaining ketolase activity.

3. The process of claim 2, wherein
(a) the Bc-enzyme is encoded by an expression improved heterologous beta-cyclase coding sequence (Bci-sequence);
(b) the Ki-enzyme or both the Ki-enzyme and the Bc-enzyme are expressed in flowers, petals, or plastids of said plant or are targeted to plastids of said plant; or
(c) the K-sequence or both the K-sequence and the Bc-sequence were modified by avoiding and/or removing signals and/or structures negatively interfering with expression efficiency in plants or plant plastids.

4-10. (canceled)

11. The process of claim 3, wherein
(a) the Ki-sequence or both the Ki- and the Bci-sequence were modified by adapting the corresponding non-improved coding sequences to the codon usage of said plant or compartment of the plant such as tissue or plastid; or
(b) the Bc-sequence or the Bci-sequence are derived from the corresponding coding sequence from a plant, from the family Solanaceae, or from the species Lycopersicon esculentum.

12. The process of claim 11, wherein at least one codon of the non-improved ketolase coding sequence (K-sequence) or both the K- and the beta-cyclase sequence (Bc-sequence) were adapted to the most abundant codon for the same amino acid of the plant or compartment of the plant such as tissue or plastid.

13-21. (canceled)

22. The process of claim 2, wherein the beta-cyclase enzyme is expressed having an amino acid sequence encoded by the corresponding coding portions comprised by a nucleotide sequence selected from SEQ ID NO: 15, 17 and 25; or coding sequences derived there from by nucleic acid substitution, addition, deletion or insertion, and encoding a beta-cyclase enzyme having a sequence identity of at least 50% with respect to the parent sequence and retaining beta-cyclase activity.

23. The process of claim 22, wherein the improved beta-cyclase sequence is selected from the corresponding coding portions comprised by a nucleotide sequence selected from SEQ ID NO: 17; or coding sequences derived there from by nucleic acid substitution, addition, deletion or insertion, and encoding a ketolase enzyme having a sequence identity of at least 50% with respect to the parent sequence and retaining beta-cyclase activity.

24. The process of claim 1, wherein a genetically modified plant is employed which comprises
(a) an expression construct comprising at least one Ki-sequence or at least one Ki-sequence and at least one Bc-sequence; or
(b) at least two expression constructs one carrying at least one Ki-sequence and the other carrying at least one Bc-sequence,
wherein the Ki-sequence and Bc-sequence are under the control of suitable regulatory elements.

25. The process of claim 24, wherein the Ki-sequence or both the Ki-sequence and the Bc-sequence are under the control of a plant specific promoter.

26-28. (canceled)

29. The process of claim 25, wherein the expression constructs further comprise the coding sequence for a transit peptide operably linked to the coding sequence of the Ki-sequence or to each of the Ki-sequence and the Bc-sequence.

30. The process of claim 29, wherein the coding sequence for the transit peptide is also expression improved.

31. An expression construct comprising
(a) at least one Ki-sequence;
(b) at least one Bc-sequence;
(c) at least one Ki-sequence and at least one Bc-sequence; or
(d) at least one of the expression improved Ki-sequence or Bci-sequence of claim 49,
wherein the Ki-sequence, Bc-sequence, and Bci-sequence are under the control of suitable regulatory elements.

32. A recombinant vector comprising at least one expression construct as defined in claim 31.

33. A recombinant microorganism comprising at least one expression construct as defined in claim 31 or a recombinant vector comprising said expression construct.

34. A genetically modified plant comprising
(a) at least one expression improved heterologous carotene ketolase coding sequence (Ki-sequence) encoding a carotene ketolase enzyme (K-enzyme);
(b) at least one expression construct as defined in claim 31;
(c) at least one vector comprising the expression construct of (b); or
(d) at least 2 expression constructs, one comprising at least one Ki-sequence and the other comprising at least one Bc-sequence.

35. The genetically modified plant of claim 34
(a) expressing carotene ketolase activity in at least one plant tissue, in at least one plastid, or in petals;
(b) having an altered carotenoid profile;
(c) containing a detectable amount of at least one ketocarotenoid in at least one plant tissue; or
(d) selected from any of the following families: Ranunculaceae, Berberidaceae, Begoniaceae, Papaveraceae, Cannabaceae, Chenopodiaceae, Cruciferae, Rosaceae, Fabaceae, Linaceae, Vitaceae, Brassiceae, Cucurbitaceae, Primulaceae, Caryophyllaceae, Amaranthaceae, Apocynaceae, Balsaminaceae, Gentianaceae, Geraniaceae, Graminae, Euphorbiaceae, Labiatae, Leguminosae, Caprifoliaceae, Oleaceae, Tropaeolaceae, Solanaceae, Lobeliaceae, Scrophulariaceae, Compositae, Asteraceae, Plumbaginaceae, Liliaceae, Amaryllidaceae, Rubiaceae, Poaceae, Polemoniaceae, Orchidaceae, Umbelliferae, Verbenaceae, Violaceae, Malvaceae, Illiaceae or Lamiaceae.

36-41. (canceled)

42. Parts or seeds of the genetically modified plant of claim 34 comprising
(a) at least one expression construct as defined in claim 31, comprising
(i) at least one Ki-sequence;
(ii) at least one Bc-sequence; or
(iii) at least one Ki-sequence and at least one Bc-sequence,
wherein the Ki-sequence and Bc-sequence are under the control of suitable regulatory elements; or
(b) at least one vector comprising the expression construct of (a).

43. A process for the preparation of a genetically modified plant, which process comprises introducing at least one expression construct as defined in claim 31 into a starting plant.

44. A process for the preparation of parts or seeds of a genetically modified plant, which process comprises introducing at least one expression construct as defined in claim 31 into a starting plant, growing the so obtained genetically modified plant, and obtaining parts thereof.

45. A process for the preparation of at least one carotenoid or ketocarotenoid, which process comprises cultivating the genetically modified plant of claim 34 under conditions which allow the expression of improved ketolase activity for a sufficient time to produce a detectable amount of at least one carotenoid or ketocarotenoid within the plant, and isolating said carotenoid or ketocarotenoid from the plant.

46-48. (canceled)

49. An expression improved carotene ketolase sequence (Ki-sequence) or an expression improved heterologous beta-cyclase coding sequence Bci-sequence wherein the Ki-sequence and Bci-sequence were modified by adapting the corresponding non-improved coding sequences to the codon usage of said plant or compartment of the plant such as tissue or plastid.

50. (canceled)

51. A method for heterologous expression of genes in plants of the genus Tagetes comprising transforming a plant with a heterologous gene functionally linked to an Antirrhinum majus ANTHIRRHINUM FIDDLEHEAD (AFI) promoter.

52. The method of claim 51, wherein the expression takes place specifically in epidermis.

53. The method of claim 51, wherein the AFI promoter comprises
A1) the nucleic acid sequence SEQ. ID. NO. of SEQ ID NO: 28;
A2) a sequence derived from the nucleic acid sequence of SEQ ID NO: 28 by substitution, insertion or deletion of nucleotides and having a sequence identity of at least 60% at the nucleic acid level with the respective nucleic acid sequence of SEQ ID NO: 28;
A3) a nucleic acid sequence which hybridizes with the nucleic acid sequence of SEQ ID NO: 28 under stringent conditions; or
A4) a functionally equivalent fragment of the sequences of A1), A2) or A3).

54. The method of claim 51, wherein the AFI promoter is functionally linked to a ketolase gene and/or a beta-cyclase gene.

55. A genetically modified plant of the genus Tagetes produced by the method of claim 51, where the genetic modification increases or causes the expression of at least one gene compared with a corresponding reference plant, and the expression or increased expression is caused by regulation of the expression of the at least one gene in the plant by the AFI promoter.

56. The genetically modified plant of claim 55, wherein the regulation of the expression of the at least one gene in the plant is achieved by
a) introducing one or more AFI promoters into the genome of the plant, so that expression of one or more endogenous genes takes place under the control of the introduced AFI promoters,
b) introducing one or more genes into the genome of the plant, so that expression of one or more of the introduced genes takes place under the control of an endogenous AFI promoter, or
c) introducing one or more nucleic acid constructs comprising at least one AFI promoter functionally linked to one or more genes to be expressed in the plant.

57. A process for producing biosynthetic products comprising cultivating the genetically modified plant of claim 55.

58. A process for producing carotenoids comprising cultivating the genetically modified plant of claim 55, wherein the genes to be expressed comprise at least one ketolase gene and/or at least one beta-cyclase gene.

59. The process of claim 58, wherein the ketolase gene is from Scenedesmus or the beta-cyclase gene is a B-gene from tomato.

60. (canceled)

61. The process of claim 58, wherein the carotenoids are astaxanthin or astaxanthin derivatives.

62. The process of claim 61, wherein the genetically modified plant or parts thereof are harvested after the cultivation, and then the carotenoids are isolated from the genetically modified plant or parts thereof.
